(19) United States
(12) Patent Application Publication
(10) Pub. No.: US 2012/0129876 A1
(43) Pub. Date: May 24, 2012

(54) [4-(6-FLUORO-7-METHYLAMINO-2,4-DIOXO-1,4-DIHYDRO-2H-QUINAZOLIN-3-YL)-PHENYL]-5-CHLORO-THIOPHEN-2-YL-SULFONYLUREA SALTS, FORMS AND METHODS RELATED THERETO

(75) Inventors: Emma Sharp, Cambridgeshire (GB); Louisa Jane Quegan, Cambridgeshire (GB); Anjali Pandey, Fremont, CA (US); Juan Wang, Foster City, CA (US); Matthew Nieder, San Francisco, CA (US); Wolin Huang, Foster City, CA (US)

(73) Assignee: Portola Pharmaceuticals, Inc., South San Francisco, CA (US)

(21) Appl. No.: 13/166,763

(22) Filed: Jun. 22, 2011

Related U.S. Application Data
(63) Continuation of application No. 12/265,699, filed on Nov. 5, 2008, now abandoned.
Application No. 12/265,699 is a continuation-in-part of application No. 12/114,742, filed on May 2, 2008.
(60) Provisional application No. 60/927,328, filed on May 2, 2007.

(51) Int. Cl. A61K 31/517; A61P 9/10; A61P 7/02; C07D 409/12; A01N 1/02

(52) U.S. Cl. 514/266.24; 544/285; 435/2

(57) ABSTRACT

The present invention provides novel sulfonylurea salts of a salt of formula (I)

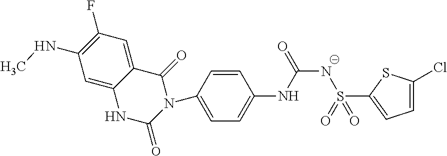

and polymorph forms thereof. The compounds in their various forms are effective platelet ADP receptor inhibitors and may be used in various pharmaceutical compositions, and are particularly effective for the prevention and/or treatment of cardiovascular diseases, particularly those diseases related to thrombosis. The invention also provides a method for preparing such compounds and forms and for preventing or treating thrombosis and thrombosis related conditions in a mammal comprising the step of administering a therapeutically effective amount of a salt of formula (I) or a pharmaceutically acceptable form thereof.

### CROSS-REFERENCE TO RELATED APPLICATIONS

The present application is a continuation of U.S. patent application Ser. No. 12/265,699, filed Nov. 5, 2008, which is a continuation-in-part of U.S. patent application Ser. No. 12/114,742, filed May 2, 2008, which application claims the benefit of priority under 35 USC 119(e) of U.S. Provisional Patent Application No. 60/927,328, filed May 2, 2007, which are herein incorporated by reference in their entirety for all purposes.

### BACKGROUND OF THE INVENTION

Thrombotic complications are a major cause of death in the industrialized world. Examples of these complications include acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura. Thrombotic and restenotic complications also occur following invasive procedures, e.g., angioplasty, carotid endarterectomy, post CABG (coronary artery bypass graft) surgery, vascular graft surgery, stent placements and insertion of endovascular devices and prostheses, and hypercoagulable states related to genetic predisposition or cancers. It is generally thought that platelet aggregates play a critical role in these events. Blood platelets, which normally circulate freely in the vasculature, become activated and aggregate to form a thrombus from disturbed blood flow caused by ruptured atherosclerotic lesions or by invasive treatments such as angioplasty, resulting in vascular occlusion. Platelet activation can be initiated by a variety of agents, e.g., exposed subendothelial matrix molecules such as collagen, or by thrombin which is formed in the coagulation cascade.

An important mediator of platelet activation and aggregation is ADP (adenosine 5′-diphosphate) which is released from blood platelets in the vasculature upon activation by various agents, such as collagen and thrombin, and from damaged blood cells, endothelium or tissues. Activation by ADP results in the recruitment of more platelets and stabilization of existing platelet aggregates. Platelet ADP receptors mediating aggregation are activated by ADP and some of its derivatives and antagonized by ATP (adenosine 5′-triphosphate) and some of its derivatives (Mills, D. C. B. (1996) Thromb. Hemost. 76:835-856). Therefore, platelet ADP receptors are members of the family of P2 receptors activated by purine and/or pyrimidine nucleotides (King, B. F., Townsend-Nicholson, A. & Burnstock, G. (1998) Trends Pharmacol. Sci. 19:506-514).

Recent pharmacological data using selective antagonists suggests that ADP-dependent platelet aggregation requires activation of at least two ADP receptors (Kunapuli, S. P. (1998), Trends Pharmacol Sci. 19:391-394; Kunapuli, S. P. & Daniel, J. L. (1998) Biochem. J. 336:513-523; Jantzen, H. M. et al. (1999) Thromb. Hemost. 81:111-117). One receptor appears to be identical to the cloned P2Y₁ receptor, mediates phospholipase C activation and intracellular calcium mobilization and is required for platelet shape change. The second platelet ADP receptor important for aggregation mediates inhibition of adenylyl cyclase. Based on its pharmacological and signaling properties this receptor has been provisionally termed P2Y_{ADP} (Fredholm, B. B. et al. (1997) TIPS 18:79-82), P2T_{AC} (Kunapuli, S. P. (1998), Trends Pharmacol. Sci. 19:391-394) or P2Ycyc (Hechier, B. et al. (1998) Blood 92, 152-159). More recently, molecular cloning of this receptor (Hollopeter, G. et al. (2001) Nature 409: 202-207) has revealed that it is a new member of the G-protein coupled family and is the target of the thienopyridine drugs ticlopidine and clopidogrel. The nomenclature given to this receptor is P2Y₁₂.

Various directly or indirectly acting synthetic inhibitors of ADP-dependent platelet aggregation with antithrombotic activity have been reported. The orally active antithrombotic thienopyridines ticlopidine and clopidogrel inhibit ADP-induced platelet aggregation, binding of radiolabeled ADP receptor agonist 2-methylthioadenosine 5′-diphosphate to platelets, and other ADP-dependent events indirectly, probably via formation of an unstable and irreversible acting metabolite (Quinn, M. J. & Fitzgerald, D. J. (1999) Circulation 100:1667-1667). Some purine derivatives of the endogenous antagonist ATP, e.g., AR-C (formerly FPL or ARL) 67085MX and AR-C69931Mx, are selective platelet ADP receptor antagonists which inhibit ADP-dependent platelet aggregation and are effective in animal thrombosis models (Humphries et al. (1995), Trends Pharmacol. Sci. 16, 179; Ingall, A. H. et al. (1999) J. Med. Chem. 42, 213-230). Novel triazolo[4,5-d]pyrimidine compounds have been disclosed as P_{2T}-antagonists (WO 99/05144). Tricyclic compounds as platelet ADP receptor inhibitors have also been disclosed in WO 99/36425. The target of these antithrombotic compounds appears to be P₂Y₁₂, the platelet ADP receptor mediating inhibition of adenylyl cyclase.

Despite these compounds, there exists a need for more effective platelet ADP receptor inhibitors. In particular, there is a need for platelet ADP receptor inhibitors having antithrombotic activity that are useful in the prevention and/or treatment of cardiovascular diseases, particularly those related to thrombosis.

In addition, while biological activity is a sine non qua for an effective drug, the compound must be capable of large scale manufacturing and the physical properties of the compound can markedly impact the effectiveness and cost of a formulated active ingredient. Salts of acidic and basic compounds can alter or improve the physical properties of a parent compound. These salt forming agents, however, must be identified empirically by the pharmaceutical chemist since there is no reliable method to predict the influence of a salt species on the behavior of a parent compound in dosage forms. Effective screening techniques, which potentially could simplify the selection process, are unfortunately absent (G. W. Radebaugh and L. J. Ravin Preformulation. In, Remington: The Science and Practice of Pharmacy; A. R. Gennaro Ed.; Mack Publishing Co. Easton, Pa., 1995; pp 1456-1457).

Amorphous and different crystalline forms (polymorphic or solvated) of salts are frequently encountered among pharmaceutically useful compounds. Polymorphism is the ability of any element or compound to crystallize in more than one lattice arrangement. Physical properties including solubility, melting point (endotherm onset in DSC analysis), density, hardness, crystal shape and stability can be different for different solid forms of the same chemical compound.

Crystalline and amorphous forms may be characterized by scattering techniques, e.g., X-ray powder diffraction, by spectroscopic methods, e.g., infra-red, solid state ¹³C and ¹⁹F nuclear magnetic resonance spectroscopy and by thermal techniques, e.g, differential scanning calorimetry (DSC) or thermogravimetric analysis (TGA). Although the intensities of peaks in the X-ray powder diffraction patterns of different batches of a polymorph may vary slightly, the peak locations are characteristic for a specific crystalline solid form. Additionally, infrared, Raman and thermal methods have been used to interpret differences between crystalline forms. Crystalline and amorphous forms may be characterized by data from the X-ray powder diffraction pattern determined in accordance with procedures which are known in the art (see J. Haleblian, J. Pharm. Sci. 1975 64:1269-1288, and J. Haleblain and W. McCrone, J. Pharm. Sci. 1969 58:911-929).

As discussed in U.S. patent application Ser. No. 11/556,490, the free acid compound of the salt of formula I (Formula II) is a potent platelet ADP receptor inhibitor. Surprisingly and unexpectedly, it was found that certain salts and crystalline forms of the present invention show improved properties including but not limited to crystallinity, thermal, hydrolytic and hygroscopic stability and purity. In addition, the salts of Formula I of the present invention are useful for the treatment of undesired thrombosis in mammals.

### SUMMARY OF THE INVENTION

In one aspect, the present invention provides a salt comprising a compound Formula I:

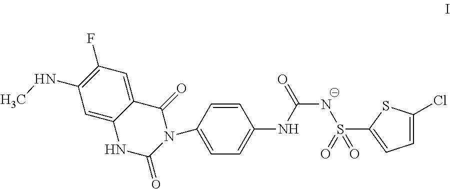

and an ion selected from the group consisting of calcium, L-lysine, ammonium, magnesium, L-arginine, tromethamine, N-ethylglucamine and N-methylglucamine.

In another aspect, the invention provides crystalline solid forms of the sodium, potassium, calcium, L-lysine, ammonium, tromethamine salts of [4-(6-fluoro-7-methylamino-2,4-dioxo-1,4-dihydro-2H-quinazolin-3-yl)-phenyl]-5-chloro-thiophen-2-yl-sulfonylurea. In another aspect, the invention provides crystalline solid forms of the magnesium salt of [4-(6-fluoro-7-methylamino-2,4-dioxo-1,4-dihydro-2H-quinazolin-3-yl)-phenyl]-5-chloro-thiophen-2-yl-sulfonylurea.

In another aspect, the invention provides pharmaceutical compositions for preventing or treating thrombosis and thrombosis related conditions in a mammal. The compositions contain a therapeutically effective amount of one or more salts of formula (I) or a pharmaceutically acceptable salt thereof and a pharmaceutically acceptable carrier or excipient. The invention further provides a method for preventing or treating thrombosis and thrombosis related conditions in a mammal by administering a therapeutically effective amount of a salt of formula (I).

In still another aspect, the present invention provides methods for preparing salts of formula (I), their crystalline solid and amorphous forms and pharmaceutical compositions for preventing or treating thrombosis and thrombosis related conditions in a mammal.

In some embodiments, the present invention provides a method for preventing or treating a condition in a mammal characterized by undesired thrombosis comprising administering to the mammal a therapeutically effective amount of a salt of Formula I or the salt of Formula I having a crystalline polymorph form including the sodium and potassium salts. In another embodiment, the condition is selected from the group consisting of acute coronary syndrome, myocardial infarction, unstable angina, refractory angina, occlusive coronary thrombus occurring post-thrombolytic therapy or post-coronary angioplasty, a thrombotically mediated cerebrovascular syndrome, embolic stroke, thrombotic stroke, transient ischemic attacks, venous thrombosis, deep venous thrombosis, pulmonary embolus, coagulopathy, disseminated intravascular coagulation, thrombotic thrombocytopenic purpura, thromboanglitis obliterans, thrombotic disease associated with heparin-induced thrombocytopenia, thrombotic complications associated with extracorporeal circulation, thrombotic complications associated with instrumentation, and thrombotic complications associated with the fitting of prosthetic devices.

In another embodiment, the present invention provides a method for inhibiting the coagulation of a blood sample comprising the step of contacting the sample with a salt comprising the salt of formula I including in a crystalline solid form.

In a further embodiment, the present invention provides a method of preparing a salt of formula I comprising contacting a base with a compound of formula II:

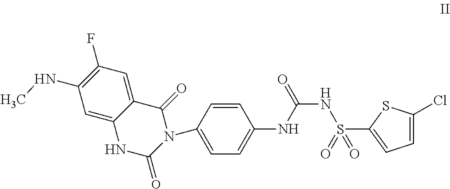

or a salt thereof under conditions to form the salt of Formula I.

In some embodiments, the conditions are nucleophilic addition conditions and comprise use of a non-polar, aprotic solvent. In some other embodiments, the solvent is a member selected from the group consisting of tetrahydrofuran, diethyl ether, dimethoxymethane, dioxane, hexane, methyl tert-butyl ether, heptane, and cyclohexane. In some embodiments, the salt of the compound of Formula II is an acid salt.

In some embodiments, the present invention provides a method of preparing a salt of formula I wherein the method is performed at a temperature of less than 10° C.

In a further embodiment, the present invention provides a method of preparing a salt of formula I wherein the compound having Formula I is afforded in a yield of at least 50%. In another embodiment, the compound having Formula I is afforded in a yield of at least 65%. In still another embodiment, the compound having Formula I is afforded in a yield of at least 75%.

In another embodiment, the present invention provides a method of making the salt of formula I on a gram scale or a kilogram scale.

### BRIEF DESCRIPTION OF THE DRAWINGS

FIG. 1 provides structure of [4-(6-fluoro-7-methylamino-2,4-dioxo-1,4-dihydro-2H-quinazolin-3-yl)-phenyl]-5-chloro-thiophen-2-yl-sulfonylurea free acid.

FIG. 2a shows an X-ray powder diffraction (XRPD) of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate. FIG. 2b shows an XRPD of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate showing peak position information.

FIG. 3a shows an XRPD of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate. FIG. 3b shows an XRPD of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate showing peak position information.

FIG. 4 shows a Fourier-transformed infrared spectra (FT-IR) of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate.

FIG. 5 shows a Fourier-transformed infrared spectra (FT-IR) of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate.

FIG. 6 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea potassium salt.

FIG. 7 provides the gravimetric vapour sorption (GVS) data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate.

FIG. 8 shows the XRPD of form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate, after the GVS experiment.

FIG. 9 provides the gravimetric vapour sorption (GVS) data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate.

FIG. 10 provides the XRPD of form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate after the GVS experiment. These results show that no phase change has occurred over the course of the GVS experiment. The change in intensity of the peak at ca. 5.4° 2θ is a preferred orientation effect.

FIG. 11 provides the differential scanning calorimetry (DSC) data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate.

FIG. 12 provides the TGA data of crystalline solid form A of [4-(6-fluoro-7-methylamino-2,4-dioxo-1,4-dihydro-2H-quinazolin-3-yl)-phenyl]-5-chloro-thiophen-2-yl-sulfonylurea potassium salt 2.5 hydrate.

FIG. 13 provides the DSC data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate.

FIG. 14 provides the TGA data of crystalline solid form B of [4-(6-fluoro-7-methylamino-2,4-dioxo-1,4-dihydro-2H-quinazolin-3-yl)-phenyl]-5-chloro-thiophen-2-yl-sulfonylurea potassium salt hemi hydrate.

FIG. 15 shows the XRPD of crystalline solid form C of [4-(6-fluoro-7-methylamino-2,4-dioxo-1,4-dihydro-2H-quinazolin-3-yl)-phenyl]-5-chloro-thiophen-2-yl-sulfonylurea potassium salt (non-stoichiometric hydrate).

FIG. 16 provides the VT XRPD results of [4-(6-fluoro-7-methylamino-2,4-dioxo-1,4-dihydro-2H-quinazolin-3-yl)-phenyl]-5-chloro-thiophen-2-yl-sulfonylurea potassium salt (form C). Form C was shown to desolvate to an amorphous phase on heating.

FIG. 17 provides the gravimetric vapour sorption (GVS) of [4-(6-fluoro-7-methylamino-2,4-dioxo-1,4-dihydro-2H-quinazolin-3-yl)-phenyl]-5-chloro-thiophen-2-yl-sulfonylurea potassium salt form C (non-stoichiometric hydrate).

FIG. 18 provides the XRPD of [4-(6-fluoro-7-methylamino-2,4-dioxo-1,4-dihydro-2H-quinazolin-3-yl)-phenyl]-5-chloro-thiophen-2-yl-sulfonylurea potassium salt non-stoichiometric hydrate (form C) re-analysis post GVS. The analysis showed the sample to be reduced in crystallinity after the GVS experiment, with some subtle changes in form.

FIG. 19 shows the TGA data of [4-(6-fluoro-7-methylamino-2,4-dioxo-1,4-dihydro-2H-quinazolin-3-yl)-phenyl]-5-chloro-thiophen-2-yl-sulfonylurea potassium salt non-stoichiometric hydrate, form C.

FIG. 20 shows the DSC data of [4-(6-fluoro-7-methylamino-2,4-dioxo-1,4-dihydro-2H-quinazolin-3-yl)-phenyl]-5-chloro-thiophen-2-yl-sulfonylurea potassium salt non-stoichiometric hydrate, form C.

FIG. 21 provides the XRPD of [4-(6-fluoro-7-methylamino-2,4-dioxo-1,4-dihydro-2H-quinazolin-3-yl)-phenyl]-5-chloro-thiophen-2-yl-sulfonylurea potassium salt (form D).

FIG. 22 shows the stability with respect to 40° C./75% RH of [4-(6-fluoro-7-methylamino-2,4-dioxo-1,4-dihydro-2H-quinazolin-3-yl)-phenyl]-5-chloro-thiophen-2-yl-sulfonylurea potassium salt (form D) by XRPD. The solid converts to an amorphous phase on storage when subjected to these conditions.

FIG. 23 provides the TGA data of [4-(6-fluoro-7-methylamino-2,4-dioxo-1,4-dihydro-2H-quinazolin-3-yl)-phenyl]-5-chloro-thiophen-2-yl-sulfonylurea potassium salt (form D).

FIG. 24 provides the DSC data of [4-(6-fluoro-7-methylamino-2,4-dioxo-1,4-dihydro-2H-quinazolin-3-yl)-phenyl]-5-chloro-thiophen-2-yl-sulfonylurea potassium salt (form D).

FIG. 25 shows the XRPD of [4-(6-fluoro-7-methylamino-2,4-dioxo-1,4-dihydro-2H-quinazolin-3-yl)-phenyl]-5-chloro-thiophen-2-yl-sulfonylurea sodium salt (form A).

FIG. 26 shows the stability with respect to 40° C./75% RH of [4-(6-fluoro-7-methylamino-2,4-dioxo-1,4-dihydro-2H-quinazolin-3-yl)-phenyl]-5-chloro-thiophen-2-yl-sulfonylurea sodium salt (form A) by XRPD. The sample converts to an amorphous phase when subjected to these conditions.

FIG. 27 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 28 shows the TGA data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 29 shows the DSC data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 30 shows the XRPD of [4-(6-fluoro-7-methylamino-2,4-dioxo-1,4-dihydro-2H-quinazolin-3-yl)-phenyl]-5-chloro-thiophen-2-yl-sulfonylurea sodium salt (form B).

FIG. 31 shows the TGA trace for crystalline solid form B of the sodium salt of [4-(6-fluoro-7-methylamino-2,4-dioxo-1,4-dihydro-2H-quinazolin-3-yl)-phenyl]-5-chloro-thiophen-2-yl-sulfonylurea.

FIG. 32 shows the XRPD of [4-(6-fluoro-7-methylamino-2,4-dioxo-1,4-dihydro-2H-quinazolin-3-yl)-phenyl]-5-chloro-thiophen-2-yl-sulfonylurea sodium salt (form C).

FIG. 33 shows the TGA data for crystalline solid form C of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 34 shows the DSC data for crystalline solid form C of [4-(6-fluoro-7-methylamino-2,4-dioxo-1,4-dihydro-2H-quinazolin-3-yl)-phenyl]-5-chloro-thiophen-2-yl-sulfonylurea sodium salt.

FIG. 35 shows the GVS of [4-(6-fluoro-7-methylamino-2,4-dioxo-1,4-dihydro-2H-quinazolin-3-yl)-phenyl]-5-chloro-thiophen-2-yl-sulfonylurea sodium salt (form C).

FIG. 36 shows the XRPD of [4-(6-fluoro-7-methylamino-2,4-dioxo-1,4-dihydro-2H-quinazolin-3-yl)-phenyl]-5-chloro-thiophen-2-yl-sulfonylurea sodium salt (form C) re-analysed after the GVs experiment.

FIG. 37 shows the XRPD of [4-(6-fluoro-7-methylamino-2,4-dioxo-1,4-dihydro-2H-quinazolin-3-yl)-phenyl]-5-chloro-thiophen-2-yl-sulfonylurea calcium salt (form A).

FIG. 38 shows the stability of [4-(6-fluoro-7-methylamino-2,4-dioxo-1,4-dihydro-2H-quinazolin-3-yl)-phenyl]-5-chloro-thiophen-2-yl-sulfonylurea calcium salt (form A) by XRPD. The sample remains stable after 3 days at 40° C./75% RH, and a further 4 days at 60° C./75% RH.

FIG. 39 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea calcium salt.

FIG. 40 shows the GVS of [4-(6-fluoro-7-methylamino-2,4-dioxo-1,4-dihydro-2H-quinazolin-3-yl)-phenyl]-5-chloro-thiophen-2-yl-sulfonylurea calcium salt (form A).

FIG. 41 shows the XRPD re-analysis of [4-(6-fluoro-7-methylamino-2,4-dioxo-1,4-dihydro-2H-quinazolin-3-yl)-phenyl]-5-chloro-thiophen-2-yl-sulfonylurea calcium salt (form A) after the GVS experiment.

FIG. 42 shows the TGA data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea calcium salt.

FIG. 43 shows the DSC data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea calcium salt.

FIG. 44 shows the XRPD of [4-(6-fluoro-7-methylamino-2,4-dioxo-1,4-dihydro-2H-quinazolin-3-yl)-phenyl]-5-chloro-thiophen-2-yl-sulfonylurea tromethamine salt (form A).

FIG. 45 shows the stability of [4-(6-fluoro-7-methylamino-2,4-dioxo-1,4-dihydro-2H-quinazolin-3-yl)-phenyl]-5-chloro-thiophen-2-yl-sulfonylurea tromethamine salt (form A) by XRPD. The sample shows some subtle changes after 3 days at 40° C./75% RH, but no further changes after 4 days at 60° C./75% RH.

FIG. 46 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea tromethamine salt.

FIG. 47 shows the TGA data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea tromethamine salt.

FIG. 48 shows the DSC data for crystalline solid form A of [4-(6-fluoro-7-methylamino-2,4-dioxo-1,4-dihydro-2H-quinazolin-3-yl)-phenyl]-5-chloro-thiophen-2-yl-sulfonylurea tromethamine salt.

FIG. 49 shows the GVS of [4-(6-fluoro-7-methylamino-2,4-dioxo-1,4-dihydro-2H-quinazolin-3-yl)-phenyl]-5-chloro-thiophen-2-yl-sulfonylurea tromethamine salt (form A).

FIG. 50 shows the XRPD of [4-(6-fluoro-7-methylamino-2,4-dioxo-1,4-dihydro-2H-quinazolin-3-yl)-phenyl]-5-chloro-thiophen-2-yl-sulfonylurea tromethamine salt (form A) re-analysed after the GVS experiment.

FIG. 51 shows the XRPD of [4-(6-fluoro-7-methylamino-2,4-dioxo-1,4-dihydro-2H-quinazolin-3-yl)-phenyl]-5-chloro-thiophen-2-yl-sulfonylurea ammonium salt (form A).

FIG. 52 shows the stability of [4-(6-fluoro-7-methylamino-2,4-dioxo-1,4-dihydro-2H-quinazolin-3-yl)-phenyl]-5-chloro-thiophen-2-yl-sulfonylurea ammonium salt (form A) by XRPD. The sample showed improvements in crystallinity on storage (at 40° C./75% RH) with subtle changes in the XRPD diffractogram.

FIG. 53 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea ammonium salt.

FIG. 54 shows the TGA trace of form A of [4-(6-fluoro-7-methylamino-2,4-dioxo-1,4-dihydro-2H-quinazolin-3-yl)-phenyl]-5-chloro-thiophen-2-yl-sulfonylurea ammonium salt.

FIG. 55 shows the DSC trace of form A of [4-(6-fluoro-7-methylamino-2,4-dioxo-1,4-dihydro-2H-quinazolin-3-yl)-phenyl]-5-chloro-thiophen-2-yl-sulfonylurea ammonium salt.

FIG. 56 shows the GVS of [4-(6-fluoro-7-methylamino-2,4-dioxo-1,4-dihydro-2H-quinazolin-3-yl)-phenyl]-5-chloro-thiophen-2-yl-sulfonylurea ammonium salt (form A).

FIG. 57 show the XRPD re-analysis of [4-(6-fluoro-7-methylamino-2,4-dioxo-1,4-dihydro-2H-quinazolin-3-yl)-phenyl]-5-chloro-thiophen-2-yl-sulfonylurea ammonium salt (form A) after the GVS experiment.

FIG. 58 shows the XRPD of [4-(6-fluoro-7-methylamino-2,4-dioxo-1,4-dihydro-2H-quinazolin-3-yl)-phenyl]-5-chloro-thiophen-2-yl-sulfonylurea ammonium salt (form B).

FIG. 59 shows the stability of [4-(6-fluoro-7-methylamino-2,4-dioxo-1,4-dihydro-2H-quinazolin-3-yl)-phenyl]-5-chloro-thiophen-2-yl-sulfonylurea ammonium salt (form B) by XRPD to storage at 60° C./75% RH.

FIG. 60 shows the TGA trace of form B of [4-(6-fluoro-7-methylamino-2,4-dioxo-1,4-dihydro-2H-quinazolin-3-yl)-phenyl]-5-chloro-thiophen-2-yl-sulfonylurea ammonium salt.

FIG. 61 shows the DSC trace of form B of [4-(6-fluoro-7-methylamino-2,4-dioxo-1,4-dihydro-2H-quinazolin-3-yl)-phenyl]-5-chloro-thiophen-2-yl-sulfonylurea ammonium salt.

FIG. 62 shows the GVS of [4-(6-fluoro-7-methylamino-2,4-dioxo-1,4-dihydro-2H-quinazolin-3-yl)-phenyl]-5-chloro-thiophen-2-yl-sulfonylurea ammonium salt (form B).

FIG. 63 shows the XRPD re-analysis of [4-(6-fluoro-7-methylamino-2,4-dioxo-1,4-dihydro-2H-quinazolin-3-yl)-phenyl]-5-chloro-thiophen-2-yl-sulfonylurea ammonium salt after the GVS experiment.

FIG. 64 shows the XRPD of [4-(6-fluoro-7-methylamino-2,4-dioxo-1,4-dihydro-2H-quinazolin-3-yl)-phenyl]-5-chloro-thiophen-2-yl-sulfonylurea L-lysine salt, amorphous form.

FIG. 65 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea L-lysine salt.

FIG. 66 shows the XRPD of [4-(6-fluoro-7-methylamino-2,4-dioxo-1,4-dihydro-2H-quinazolin-3-yl)-phenyl]-5-chloro-thiophen-2-yl-sulfonylurea magnesium salt (form A).

FIG. 67 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea magnesium salt.

FIG. 68 shows the TGA trace of form A of [4-(6-fluoro-7-methylamino-2,4-dioxo-1,4-dihydro-2H-quinazolin-3-yl)-phenyl]-5-chloro-thiophen-2-yl-sulfonylurea magnesium salt.

FIG. 69 shows the XRPD of [4-(6-fluoro-7-methylamino-2,4-dioxo-1,4-dihydro-2H-quinazolin-3-yl)-phenyl]-5-chloro-thiophen-2-yl-sulfonylurea L-arginine salt, amorphous form.

FIG. 70 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea L-arginine salt.

FIG. 71 shows the XRPD of [4-(6-fluoro-7-methylamino-2,4-dioxo-1,4-dihydro-2H-quinazolin-3-yl)-phenyl]-5-chloro-thiophen-2-yl-sulfonylurea N-ethylglucamine salt (amorphous form).

FIG. 72 shows the XRPD of [4-(6-fluoro-7-methylamino-2,4-dioxo-1,4-dihydro-2H-quinazolin-3-yl)-phenyl]-5-chloro-thiophen-2-yl-sulfonylurea N-methylglucamine salt (amorphous form).

FIG. 73 shows the ¹H NMR spectrum for the [4-(6-fluoro-7-methylamino-2,4-dioxo-1,4-dihydro-2H-quinazolin-3-yl)-phenyl]-5-chloro-thiophen-2-yl-sulfonylurea N-methylglucamine salt.

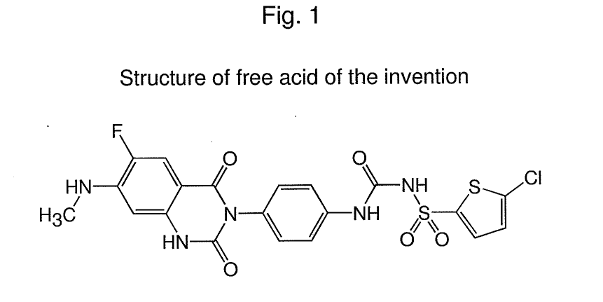

### DETAILED DESCRIPTION OF THE INVENTION

The present invention involves sulfonylurea compounds and their derivatives and crystalline solid and amorphous forms thereof, and their preparation. A selection of salts of [4-(6-fluoro-7-methylamino-2,4-dioxo-1,4-dihydro-2H-quinazolin-3-yl)-phenyl]-5-chloro-thiophen-2-yl-sulfonylurea have been isolated as crystalline solids of high purity. The salts of the present invention are useful for the treatment and prevention of undesired thrombosis and thrombosis related conditions in mammals.

### I. Definitions

In accordance with the present invention and as used herein, the following terms are defined with the following meanings, unless explicitly stated otherwise.

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “about” as used herein means variation one might see in measurements taken among different instruments, samples, and sample preparations. Such variation may include, for instance, colligative properties for thermal measurements. Typical variation among different X-ray diffractometers and sample preparations for crystalline solid forms is on the order of 0.2 °2θ. Typical variation for Raman and IR spectrometers is on the order of twice the resolution of the spectrometer. The resolution of the spectrometer used was about 2 cm⁻¹.

The term “solvate” as used herein means a compound of the invention or a salt, thereof, that further includes a stoichiometric or non-stoichiometric amount of a solvent which forms part of the crystal lattice by either non-covalent binding or by occupying a hole in the crystal lattice.

The term “hydrate” as used herein means a compound of the invention or a salt thereof, that further includes a stoichiometric or non-stoichiometric amount of water which forms part of the crystal lattice by either non-covalent bonding or by occupying a hole in the crystal lattice. Hydrates are formed by the combination of one or more molecules of water with one of the substances in which the water retains its molecular state as H₂O, such combination being able to form one or more hydrates.

The term “anhydrous” as used herein means a compound of the invention or a salt thereof that does not contain solvent in the crystal lattice.

The term “drying” as used herein means a method of removing solvent and/or water from a compound of the invention which, unless otherwise specified, may be done at atmospheric pressure or under reduced pressure and with or without heating until the level of solvent and/or water contained reached an acceptable level.

The term “polymorphs” as used herein means crystal structures in which a compound can crystallize in different crystal packing arrangements, all of which have the same elemental composition. Different crystal forms can have different X-ray diffraction patterns, infrared spectra, melting points/endotherm onset and maximums, density hardness, crystal shape, optical and electrical properties, stability and solubility. Recrystallization solvent, rate of crystallization, storage temperature, and other factors may effect which crystal form is generated.

The term “solid form” as used herein means crystal structures in which compounds can crystallize in different packing arrangements. Solid forms include polymorphs, hydrates, and solvates as those terms are used in this invention. Different solid forms, including different polymorphs, of the same compound may exhibit different x-ray powder diffraction patterns and different spectra including infra-red, Raman, DSC and solid-state NMR. Their optical, electrical, stability, and solubility properties may also differ.

The term “characterize” as used herein means to select data from an analytical measurement such as X-ray powder diffraction, DSC, infra-red spectroscopy, Raman spectroscopy, and/or solid-state NMR to distinguish one solid form of a compound from other solid forms of a compound.

The term “mammal” includes, without limitation, humans, domestic animals (e.g., dogs or cats), farm animals (cows, horses, or pigs), monkeys, rabbits, mice, and laboratory animals.

The term “alkyl” refers to saturated aliphatic groups including straight-chain, branched-chain and cyclic groups having the number of carbon atoms specified, or if no number is specified, having up to about 12 carbon atoms. Examples of alkyl groups include methyl, ethyl, n-propyl, isopropyl, n-butyl, t-butyl, isobutyl, sec-butyl, n-pentyl, n-hexyl, n-heptyl, n-octyl, and the like.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. For brevity, the term C₁₋₆alkylamino is meant to include straight chain, branched or cyclic alkyl groups or combinations thereof, such as methyl, ethyl, 2-methylpropyl, cyclobutyl and cyclopropylmethyl.

The term “C₁-C₆ alkylamino” or “C₁₋₆ alkylamino” as used herein refers to an amino moiety attached to the remainder of the molecule whereby the nitrogen is substituted with one or two C₁₋₆ alkyl substituents, as defined above.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄ haloalkyl” is mean to include trifluoromethyl, 2,2,2-trifluoroethyl, 4-chlorobutyl, 3-bromopropyl, and the like.

The term “pharmaceutically acceptable derivatives” is meant to include salts of the active compounds which are prepared with relatively non-toxic acids or bases, depending on the particular substituents found on the compounds described herein. When compounds of the present invention contain relatively acidic functionalities, base addition salts can be obtained by contacting the neutral form of such compounds with a sufficient amount of the desired base, either neat or in a suitable inert solvent. Examples of pharmaceutically acceptable base addition salts include those derived from inorganic bases such as sodium, potassium, lithium, ammonium, calcium, magnesium, iron, zinc, copper, manganese, aluminum salts and the like. Particularly preferred are the potassium, sodium, calcium, ammonium and magnesium salts. Salts derived from pharmaceutically acceptable organic non-toxic bases include salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, 2-diethylaminoethanol, tromethamine, trimethamine, dicyclohexylamine, caffeine, procaine, hydrabamine, choline, betaine, ethylenediamine, glucosamine, N-ethylglucamine, N-methylglucamine, theobromine, purines, piperazine, piperidine, N-ethylpiperidine, polyamine resins, amino acids such as lysine, arginine, histidine, and the like. Particularly preferred organic non-toxic bases are L-amino acids, such as L-lysine and L-arginine, tromethamine, N-ethylglucamine and N-methylglucamine. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively non-toxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19; Bundgaard, H., ed., Design of Prodrugs (Elsevier Science Publishers, Amsterdam 1985)). Certain specific compounds of the present invention contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present invention.

In addition to salt forms, the term “pharmaceutically acceptable derivatives” is meant to include compounds which are in a prodrug form. “Prodrugs” of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent (see Bundgaard, H., ed., Design of Prodrugs (Elsevier Science Publishers, Amsterdam 1985)).

“Pharmaceutically acceptable ester” refers to those esters which retain, upon hydrolysis of the ester bond, the biological effectiveness and properties of the carboxylic acid or alcohol and are not biologically or otherwise undesirable. For a description of pharmaceutically acceptable esters as prodrugs, see Bundgaard, H., supra. These esters are typically formed from the corresponding carboxylic acid and an alcohol. Generally, ester formation can be accomplished via conventional synthetic techniques. (See, e.g., March Advanced Organic Chemistry, 3rd Ed., p. 1157 (John Wiley & Sons, New York 1985) and references cited therein, and Mark et al., Encyclopedia of Chemical Technology, (1980) John Wiley & Sons, New York). The alcohol component of the ester will generally comprise: (i) a C₂-C₁₂ aliphatic alcohol that can or can not contain one or more double bonds and can or can not contain branched carbons; or (ii) a C₇-C₁₂ aromatic or heteroaromatic alcohols. The present invention also contemplates the use of those compositions which are both esters as described herein and at the same time are the pharmaceutically acceptable acid addition salts thereof.

“Pharmaceutically acceptable amide” refers to those amides which retain, upon hydrolysis of the amide bond, the biological effectiveness and properties of the carboxylic acid or amine and are not biologically or otherwise undesirable. For a description of pharmaceutically acceptable amides as prodrugs, see, Bundgaard, H., ed., supra. These amides are typically formed from the corresponding carboxylic acid and an amine. Generally, amide formation can be accomplished via conventional synthetic techniques. See, e.g., March et al., Advanced Organic Chemistry, 3rd Ed., p. 1152 (John Wiley & Sons, New York 1985), and Mark et al., Encyclopedia of Chemical Technology, (John Wiley & Sons, New York 1980). The present invention also contemplates the use of those compositions which are both amides as described herein and at the same time are the pharmaceutically acceptable acid addition salts thereof.

The term “pharmaceutically acceptable derivatives” is also meant to include compounds of the present invention which can exist in unsolvated forms as well as solvated forms, including hydrated forms. In general, the solvated forms are equivalent to unsolvated forms and are intended to be encompassed within the scope of the present invention. Certain compounds of the present invention may exist in multiple crystalline or amorphous forms. In general, all physical forms are equivalent for the uses contemplated by the present invention and are intended to be within the scope of the present invention.

Certain compounds of the present invention possess asymmetric carbon atoms (optical centers) or double bonds; the racemates, diastereomers, geometric isomers and individual isomers (e.g., separate enantiomers) are all intended to be encompassed within the scope of the present invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds of the present invention, whether radioactive or not, are intended to be encompassed within the scope of the present invention.

“Biological property” for the purposes herein means an in vivo effector or antigenic function or activity that is directly or indirectly performed by a compound of this invention that are often shown by in vitro assays. Effector functions include receptor or ligand binding, any enzyme activity or enzyme modulatory activity, any carrier binding activity, any hormonal activity, any activity in promoting or inhibiting adhesion of cells to an extracellular matrix or cell surface molecules, or any structural role. Antigenic functions include possession of an epitope or antigenic site that is capable of reacting with antibodies raised against it.

The term “treatment” or “treating” means any treatment of a disease or disorder in a subject, such as a mammal, including:

preventing or protecting against the disease or disorder, that is, causing the clinical symptoms not to develop;
inhibiting the disease or disorder, that is, arresting or suppressing the development of clinical symptoms; and/or
relieving the disease or disorder that is, causing the regression of clinical symptoms.

As used herein, the term “preventing” refers to the prophylactic treatment of a patient in need thereof. The prophylactic treatment can be accomplished by providing an appropriate dose of a therapeutic agent to a subject at risk of suffering from an ailment, thereby substantially averting onset of the ailment.

It will be understood by those skilled in the art that in human medicine, it is not always possible to distinguish between “preventing” and “suppressing” since the ultimate inductive event or events may be unknown, latent, or the patient is not ascertained until well after the occurrence of the event or events. Therefore, as used herein the term “prophylaxis” is intended as an element of “treatment” to encompass both “preventing” and “suppressing” as defined herein. The term “protection,” as used herein, is meant to include “prophylaxis.”

The term “therapeutically effective amount” refers to that amount of a salt of this invention, typically delivered as a pharmaceutical composition, that is sufficient to effect treatment, as defined herein, when administered to a subject in need of such treatment. The therapeutically effective amount will vary depending upon the subject and disease condition being treated, the weight and age of the subject, the severity of the disease condition, the particular compound chosen, the dosing regimen to be followed, timing of administration, the manner of administration and the like, all of which can be determined readily by one of ordinary skill in the art.

As used herein, the term “condition” refers to a disease state for which the compounds, compositions and methods of the present invention are being used against.

As used herein, the term “ADP-mediated disease or condition” and the like refers to a disease or condition characterized by less than or greater than normal, ADP activity. A ADP-mediated disease or condition is one in which modulation of ADP results in some effect on the underlying condition or disease (e.g., a ADP inhibitor or antagonist results in some improvement in patient well-being in at least some patients).

As used herein, the term “blood sample” refers to whole blood taken from a subject, or any fractions of blood including plasma or serum.

In the compounds of this invention, carbon atoms bonded to four non-identical substituents are asymmetric. Accordingly, the compounds may exist as diastereoisomers, enantiomers or mixtures thereof. The syntheses described herein may employ racemates, enantiomers or diastereomers as starting materials or intermediates. Diastereomeric products resulting from such syntheses may be separated by chromatographic or crystallization methods, or by other methods known in the art. Likewise, enantiomeric product mixtures may be separated using the same techniques or by other methods known in the art. Each of the asymmetric carbon atoms, when present in the compounds of this invention, may be in one of two configurations (R or S) and both are within the scope of the present invention.

### II. Free Acid Compounds

Compounds of formula (II) include the compound having the formula:

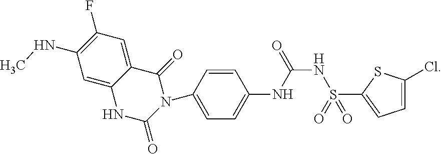

### III. Preparation of Free Acid Compounds

Scheme 1 illustrates a method of preparing certain compounds of formulas I and II wherein Ar is phenylene.

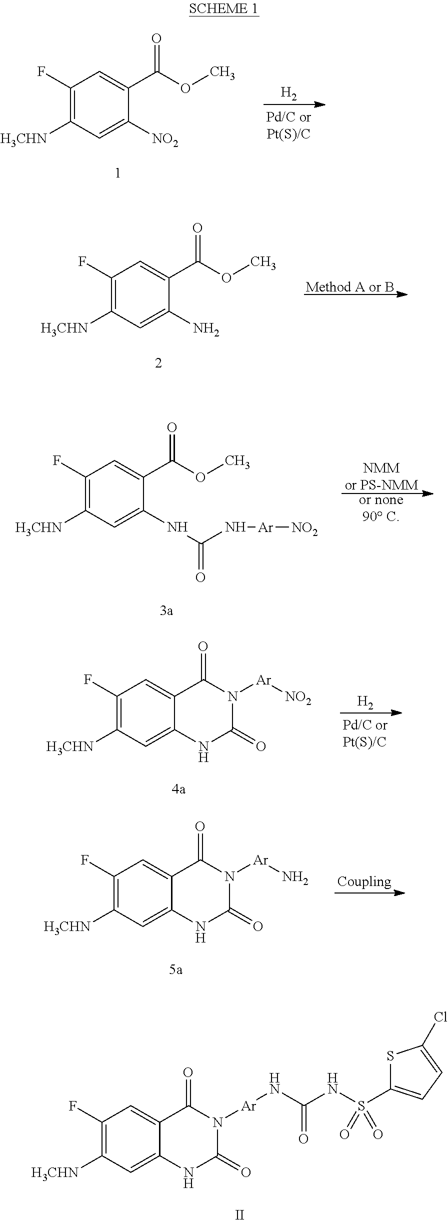

A compound of formula II can be prepared by reducing 2-nitro-benzoic acid methyl ester compound 1 by procedures known to one skilled in the art to yield aniline 2. (See also published patent application US 2002/077486). For example, a method of nitro group reduction can be carried out by hydrogenation. The hydrogenation is carried out with a suitable catalyst (e.g., 10% Pd/C or Pt(s)/C) under hydrogen and in an appropriate solvent, typically in an alcohol, preferably ethanol at room temperature. Treating compound 2 with appropriately substituted aryl isocyanate (Method A) provides intermediate urea 3a. Alternatively, urea 3a can be formed by treating compound 2 with triphosgene in the presence of a base such as triethylamine or diisopropylethylamine in an inert solvent such as THF, dichloromethane and MeCN at appropriate temperature, preferably at 20° C., followed by substituted aniline (Method B). Urea 3a, prepared by Method A or Method B typically without further purification can be subjected to thermal or base (such as N-methyl morpholine (NMM) or polystyrene-NMM (PS-NMM) induced ring closure to provide quinazolinedione 4a. The nitro group of compound 4a can be reduced by procedures known to one skilled in the art to yield free amino group. For example, a method of reduction can be carried out by hydrogenation, with a suitable catalyst (e.g., 10% palladium on carbon) in an appropriate solvent, typically an alcohol. The formation of sulfonylurea linkage can be accomplished by treating the reduced product aniline 5a with a pre-mixed solution of substituted thiophene-2-sulfonamide, N,N′-disuccinimidyl carbonate and tetramethylguanidine in dichloromethane, followed by treatment with TFA in dichloromethane at room temperature to afford the sulfonylurea of formula II. Alternatively, the sulfonylurea linkage can be formed by reacting the aniline 5a and 5-Chloro-thiophene-2-sulfonyl ethylcarbamate in suitable solvents, which include, but are not limited to, toluene, acetonitrile, 1,4-dioxane and DMSO.

Scheme 2 illustrates an alternative method of preparing compounds of Formula II wherein for example L¹ is halogen, alkylsulfonate, haloalkylsulfonate and arylsulfonate.

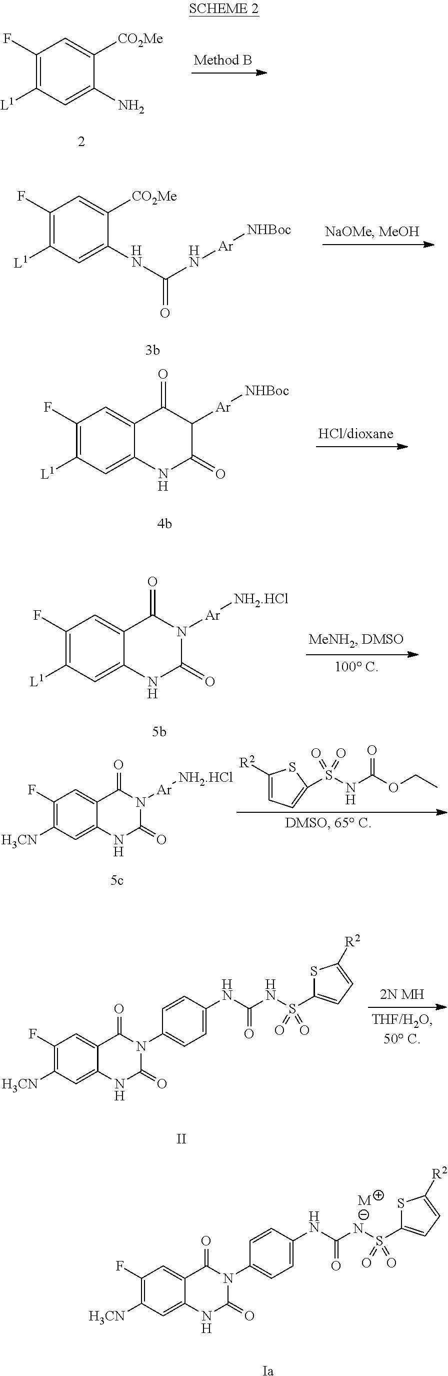

The urea 3b can be prepared by treating compound 2 with triphosgene or p-nitrophenyl chloroformate in the presence of a base, such as triethylamine and/or diisopropylethylamine, in an inert solvent, such as THF, dichloromethane and/or MeCN, at an appropriate temperature, typically at about 20° C., followed by treatment with an appropriately protected aniline (Method B). Urea 3b, typically without further purification, can be subjected to base induced ring closure to provide intermediate quinazolinedione 4b. The protecting group of compound 4b can be removed using standard techniques appropriate for the protecting group used. For example a BOC protecting group can be removed by treating compound 4b with 4N HCl in dioxane. The C-7 fluoro of compound 5b is then displaced by treatment with methylamine in DMSO at about 120° C. to afford aniline 5c. The preparation of target sulfonylurea II can be accomplished by treating aniline 5c with 5-chloro-thiophene-2-sulfonyl ethylcarbamate in an appropriate solvent, such as dimethyl sulfoxide, dioxane and/or acetonitrile with heating. Treatment of a compound of the invention with an acid or base may form, respectively, a pharmaceutically acceptable acid addition salt and a pharmaceutically acceptable base addition salt, each as defined herein. Various inorganic and organic acids and bases known in the art including those defined herein may be used to effect the conversion to the salt.

Scheme 3 illustrates an alternative method of preparing compounds of Formula II wherein for example L¹ is halogen, alkylsulfonate, haloalkylsulfonate and arylsulfonate and M is K.

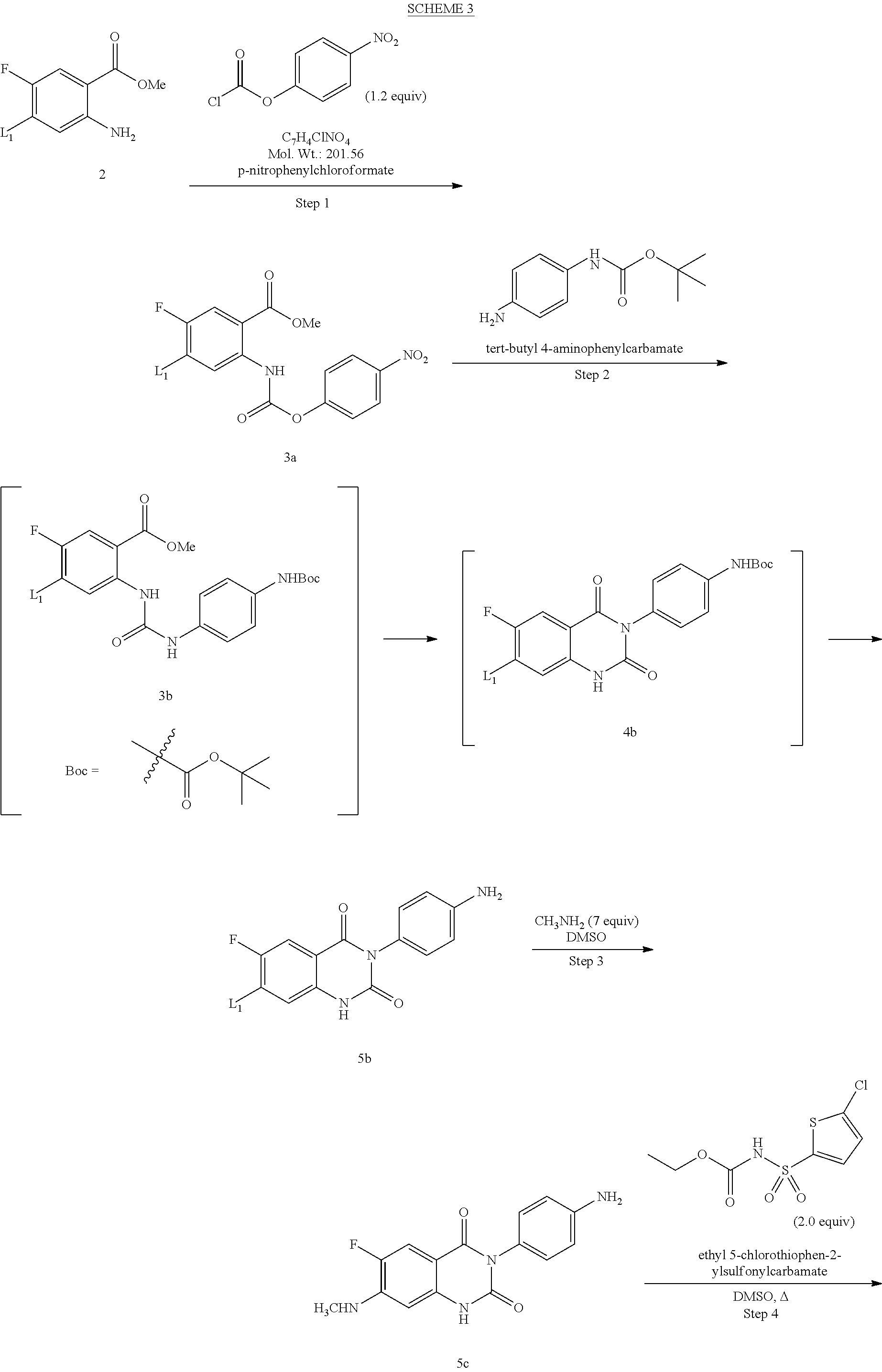

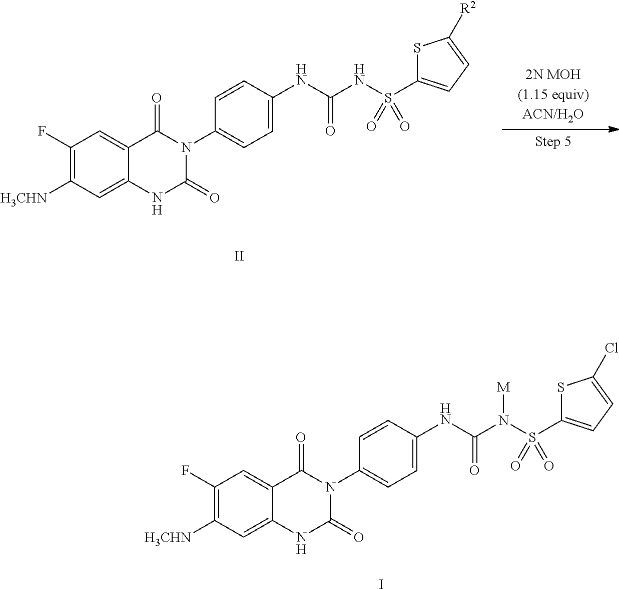

The quinazolinedione 5b can be prepared by treating compound 2 with p-nitrophenylchloroformate, in an inert solvent, such as THF, dichloromethane and/or MeCN, at an appropriate temperature, typically at about 20° C., followed by treatment with an appropriately protected aniline (Method B). The C-7 fluoro of compound 5b is then displaced by treatment with methylamine in DMSO at about 120° C. to afford aniline 5c. The preparation of target sulfonylurea II can be accomplished by treating aniline 5c with 5-chloro-thiophene-2-sulfonyl ethylcarbamate in an appropriate solvent, such as dimethyl sulfoxide, dioxane and/or acetonitrile with heating. According to the invention, compounds of formula (I) may be further treated to form pharmaceutically acceptable salts e.g. I. Treatment of a compound of the invention with an acid or base may form, respectively, a pharmaceutically acceptable acid addition salt and a pharmaceutically acceptable base addition salt, each as defined above. Various inorganic and organic acids and bases known in the art including those defined herein may be used to effect the conversion to the salt.

Compounds of formula II may be isolated using typical isolation and purification techniques known in the art, including, for example, chromatographic and recrystallization methods.

### IV. Preparation of the Salts of Formula I

According to one embodiment of the invention, compounds of formula II may be further treated to form pharmaceutically acceptable salts. Treatment of a compound of the invention with an acid or base may form, respectively, a pharmaceutically acceptable acid addition salt and a pharmaceutically acceptable base addition salt, each as defined above. These salts will preferably provide the requisite crystallinity, thermal, hydrolytic and hygroscopic stability and purity. Various inorganic and organic acids and bases including those defined herein may be used to effect the conversion to the salt by those known in the art. In one embodiment, the salts include but are not limited to, sodium and potassium salts. In another embodiment, the salts include but are not limited to, calcium, L-lysine, ammonium, magnesium, L-arginine, tromethamine, N-ethylglucamine and N-methylglucamine salts. One of skill in the art will recognize that other bases can be used to make salts comprising the compound of Formula I that are useful in the present invention. It is also contemplated that salts of the invention can be readily converted to other salts of the invention.

To assess the thermal and hydrolytic stability of the salt, tests known to those of skill in the art are performed. These tests are more thoroughly discussed below.

A number of methods are useful for the preparation of the salts described above and are known to those skilled in the art. For example, reaction of the compound of Formula II with one or more molar equivalents of the desired base in a solvent or solvent mixture in which the salt is insoluble, or in a solvent like water after which the solvent is removed by evaporation, distillation or freeze drying. Alternatively, the compound of Formula II may be passed over an ion exchange resin to form the desired salt or one salt form of the product may be converted to another using the same general process.

The salts of Formula I can be prepared according to any of several different methodologies, either on a gram scale (<1 kg) or a kilogram scale (>1 kg).

A variety of solvents can be used for the method of the present invention as described above including but not limited to a non-polar, aprotic solvent such as tetrahydrofuran (THF), diethyl ether, dimethoxymethane, dioxane, hexane, methyl tert-butyl ether, heptane, and cyclohexane. In addition, the formation of the urea can be carried at temperatures below 10° C. One of skill in the art will recognize that the methods of the present invention can be practiced using various other solvents, reagents, and reaction temperatures.

The salts of Formula I can be prepared using the method of the present invention in yields greater than 50%. In some instances, the compound of Formula I can be prepared in yields greater than 65%. In other instances, the compound of Formula I can be prepared in yields greater than 75%. One of skill in the art will recognize that the salts of Formula I can be prepared via other chemical methodologies on both a gram and kilogram scale.

The invention also provides pharmaceutically acceptable isomers, hydrates, and solvates of compounds of formula (I). Compounds of formula (I) may also exist in various isomeric and tautomeric forms including pharmaceutically acceptable salts, hydrates and solvates of such isomers and tautomers. For example, while some compounds are provided herein as dihydrates having two molecules of water per molecule of the compound of formula II, the present invention also provides compounds that are anhydrous, hemihydrates, monohydrates, trihydrates, sesquihydrates, and the like.

### IV. Crystalline Solid and Amorphous Embodiments of the Invention and their Preparation

The present invention also provides crystalline solid and/or amorphous salts of [4-(6-fluoro-7-methylamino-2,4-dioxo-1,4-dihydro-2H-quinazolin-3-yl)-phenyl]-5-chloro-thiophen-2-yl-sulfonylurea and processes for their preparation and pharmaceutical compositions comprising these forms. The salts have the following general formula:

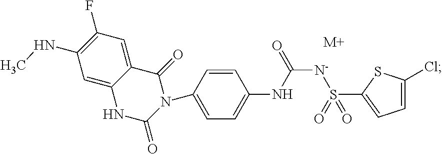

wherein M is an ion selected from the group consisting of: calcium, L-lysine, ammonium, magnesium, L-arginine, tromethamine, N-ethylglucamine and N-methylglucamine. In other embodiments, M is selected from sodium or potassium. The different crystalline forms of the same compound can have an impact on one or more physical properties, such as stability, solubility, melting point, bulk density, flow properties, bioavailability, etc.

In developing a process for production of an active pharmaceutical ingredient (API), two factors are of great importance: the impurity profile and the crystal morphology of the compound. The results from the initial isolation and crystallization work showed a profile of [4-(6-fluoro-7-methylamino-2,4-dioxo-1,4-dihydro-2H-quinazolin-3-yl)-phenyl]-5-chloro-thiophen-2-yl-sulfonylurea of 99.6%. Preferably the API has levels of impurities below 0.2% and is in the most thermodynamically stable crystalline solid form. The isolation and crystallization work indicated that there was an amorphous phase and at least four crystalline solid forms of the potassium salt of [4-(6-fluoro-7-methylamino-2,4-dioxo-1,4-dihydro-2H-quinazolin-3-yl)-phenyl]-5-chloro-thiophen-2-yl-sulfonylurea (designated as form A, B, C and D), an amorphous phase and at least three crystalline solid forms of the sodium salt of [4-(6-fluoro-7-methylamino-2,4-dioxo-1,4-dihydro-2H-quinazolin-3-yl)-phenyl]-5-chloro-thiophen-2-yl-sulfonylurea (designated as form A, B and C), at least two crystalline solid forms of the calcium salt of [4-(6-fluoro-7-methylamino-2,4-dioxo-1,4-dihydro-2H-quinazolin-3-yl)-phenyl]-5-chloro-thiophen-2-yl-sulfonylurea (designated as form A and B), at least two crystalline solid forms of the ammonium salt of [4-(6-fluoro-7-methylamino-2,4-dioxo-1,4-dihydro-2H-quinazolin-3-yl)-phenyl]-5-chloro-thiophen-2-yl-sulfonylurea (designated as form A and B), at least one amorphous form of the L-lysine salt of [4-(6-fluoro-7-methylamino-2,4-dioxo-1,4-dihydro-2H-quinazolin-3-yl)-phenyl]-5-chloro-thiophen-2-yl-sulfonylurea, at least one crystalline solid forms of the magnesium salt of [4-(6-fluoro-7-methylamino-2,4-dioxo-1,4-dihydro-2H-quinazolin-3-yl)-phenyl]-5-chloro-thiophen-2-yl-sulfonylurea (designated as form A), at least one crystalline solid form of the tromethamine salt of [4-(6-fluoro-7-methylamino-2,4-dioxo-1,4-dihydro-2H-quinazolin-3-yl)-phenyl]-5-chloro-thiophen-2-yl-sulfonylurea (designated as form A), and at least one amorphous form of the L-arginine salt, the N-ethylglucamine salt and the N-methylglucamine salt of [4-(6-fluoro-7-methylamino-2,4-dioxo-1,4-dihydro-2H-quinazolin-3-yl)-phenyl]-5-chloro-thiophen-2-yl-sulfonylurea.

The solid forms of the invention may be described by one or more of several techniques including X-ray powder diffraction, Raman spectroscopy, IR spectroscopy, and thermal methods. Further, combinations of such techniques may be used to describe the invention. For example, one or more X-ray powder diffraction patterns combined with one or more Raman spectrum may be used to describe one or more solid forms of the invention in a way that differentiates it from the other solid forms.

Although it characterizes a form, it is not necessary to rely only upon an entire diffraction pattern or spectrum to characterize a solid form. Those of ordinary skill in the pharmaceutical arts recognize that a subset of a diffraction pattern or spectrum may be used to characterize a solid form provided that subset distinguishes the solid form from the other forms being characterized. Thus, one or more X-ray powder diffraction pattern alone may be used to characterize a solid form. Likewise, one or more IR spectrum alone or Raman spectrum alone may be used to characterize a solid form. Such characterizations are done by comparing the X-ray, Raman, and IR data amongst the forms to determine characteristic peaks.

One may also combine data from other techniques in such a characterization. Thus, one may rely upon one or more x-ray powder diffraction pattern and for example, Raman or IR data, to characterize a form. For example, if one or more X-ray diffraction peak characterize a form, one could also consider Raman or IR data to characterize the form. It is sometimes helpful to consider Raman data, for example, in pharmaceutical formulations.

The solid forms were isolated by using different crystallization conditions. For the potassium salt, (1) crystalline form A was isolated after crystallization of the crude wet-cake from methanol and drying the crude wet-cake to effect solvent removal, (2) crystalline solid form B was formed from crystallization from EtOH/H₂O or by trituration with methanol, (3) crystalline solid form C was formed through grinding or suspending form B in water, or by suspending the amorphous potassium salt in water at ambient conditions it converted to form C within 16 hours. Form D could also be formed from crystallization from KOH in THF.

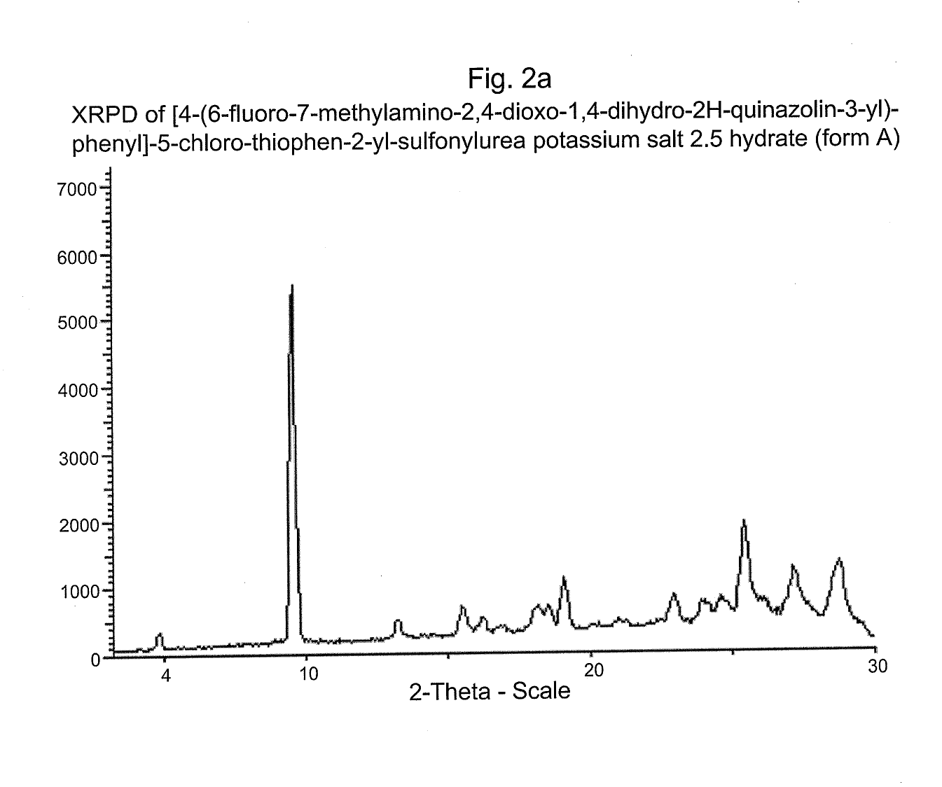

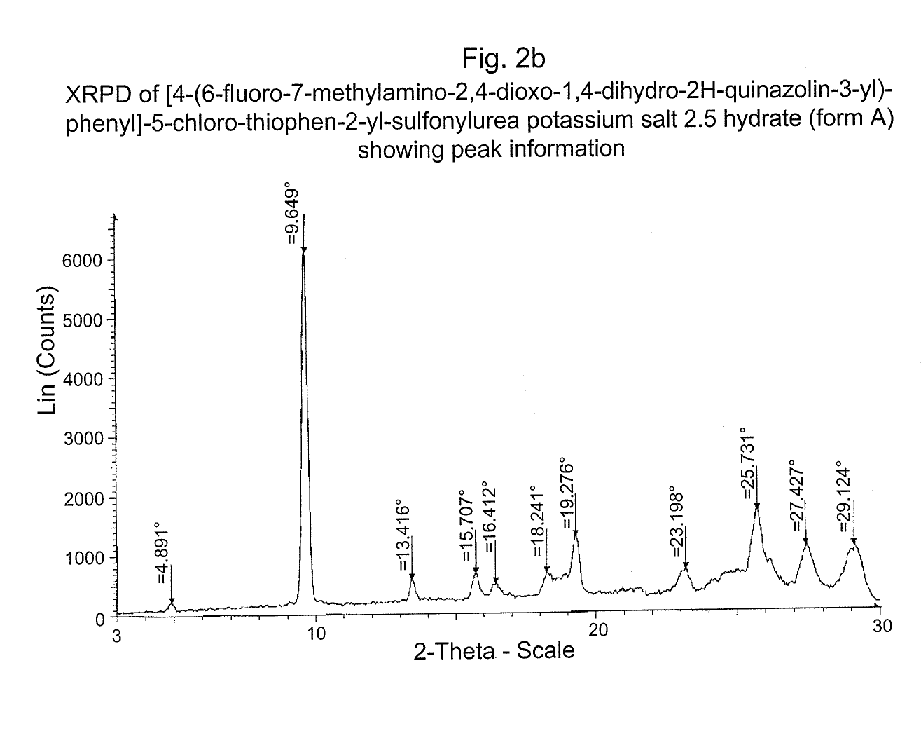

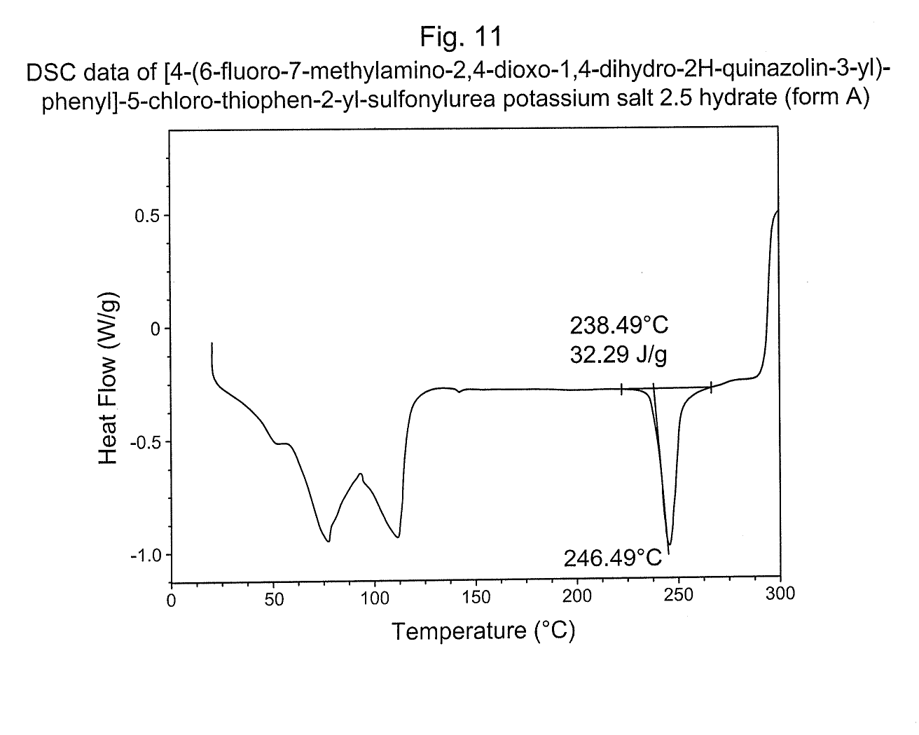

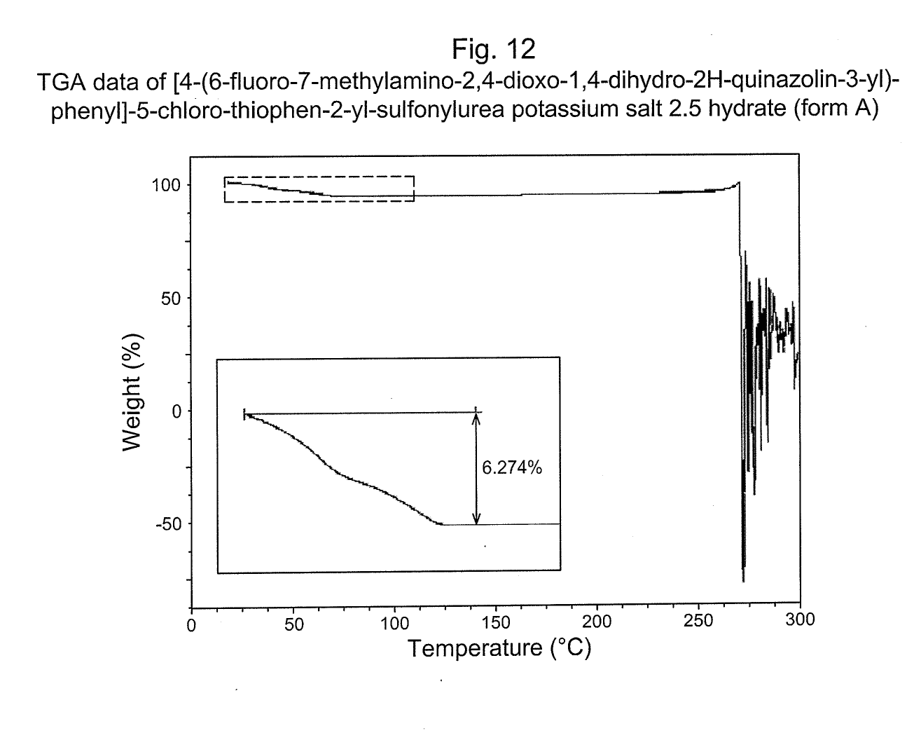

The potassium salt was suspended in methanol and then heated until a clear solution was observed. This was followed by cooling and the resulting crystalline solid was isolated and dried at room temperature under reduced pressure to give crystalline solid potassium salt form A. Form A is a mono potassium salt 2.5 hydrate. Form B is a mono potassium salt hemi hydrate. FIGS. 11 and 2 respectively show the DSC trace and the X-ray powder pattern for the crystalline solid form A. Differential scanning calorimetry (DSC) of form A of [4-(6-fluoro-7-methylamino-2,4-dioxo-1,4-dihydro-2H-quinazolin-3-yl)-phenyl]-5-chloro-thiophen-2-yl-sulfonylurea potassium salt defined a melt of dehydrated salt at 238° C. A large decomposition peak was recorded, onset temperature approximately 300° C.

In the X-ray powder diffraction pattern, the peaks at about 9.5 and 25.5 are the main features of the pattern (for a discussion of the theory of X-ray powder diffraction patterns see “X-ray diffraction procedures” by H. P. Klug and L. E. Alexander, J. Wiley, New York (1974)). The peaks at about 9.7° 2θ and 25.7° 2θ characterize form A with respect to form B because form B does not have peaks to within 0.2° 2θ, twice the approximate precision of X-ray powder diffraction peaks, of the two form A peaks. Because the typical variation in any given X-ray powder diffraction peak is on the order of 0.2° 2θ, when selecting peaks to characterize a polymorph, one selects peaks that are at least twice that value (i.e., 0.4°θ) from a peak from another polymorph. Thus, in a particular polymorph X-ray pattern, a peak that is at least 0.4°θ from a peak in another polymorph is eligible to be considered as a peak that can either alone or together with another peak be used to characterize that polymorph. Tables 1 and 2 identify the main peaks of forms A and B. From that list, one sees that the peak at about 25.7° 2θ (on the table listed as 25.73 °2θ), when taken to one decimal point, is greater than 0.2° 2θ away from any peak in forms B. Thus, the peak at about 25.7° 2θ can be used to distinguish form A from form B. The peak at about 9.7° 2θ (9.65 °2θ in Table 1) is the most intense peak in the form A X-ray powder diffraction pattern of FIG. 2 and is more than 0.2 °2θ away from any peak in form B. Thus, the form A peaks at about 9.7°2θ and 25.7 °2θ characterize form A with respect to form B. The solid form isolated at this stage in the process contained about 2.5 molecules of water to one molecule of salt.

TABLE 1
Potassium salt form A XRPD Peak (°2θ) and % Intensity
Listing, data tabulated from FIG. 2b.

| Angle/2-Theta ° | Intensity | Intensity/% |
|---|---|---|
| 4.89 | 213.00 | 3.50 |
| 9.65 | 6118.00 | 100.00 |
| 13.42 | 569.00 | 9.30 |
| 15.71 | 661.00 | 10.80 |
| 16.41 | 488.00 | 8.00 |
| 18.24 | 661.00 | 10.80 |
| 19.28 | 1270.00 | 20.80 |
| 23.20 | 691.00 | 11.30 |
| 25.73 | 1697.00 | 27.70 |
| 27.43 | 1087.00 | 17.80 |
| 29.12 | 1067.00 | 17.40 |

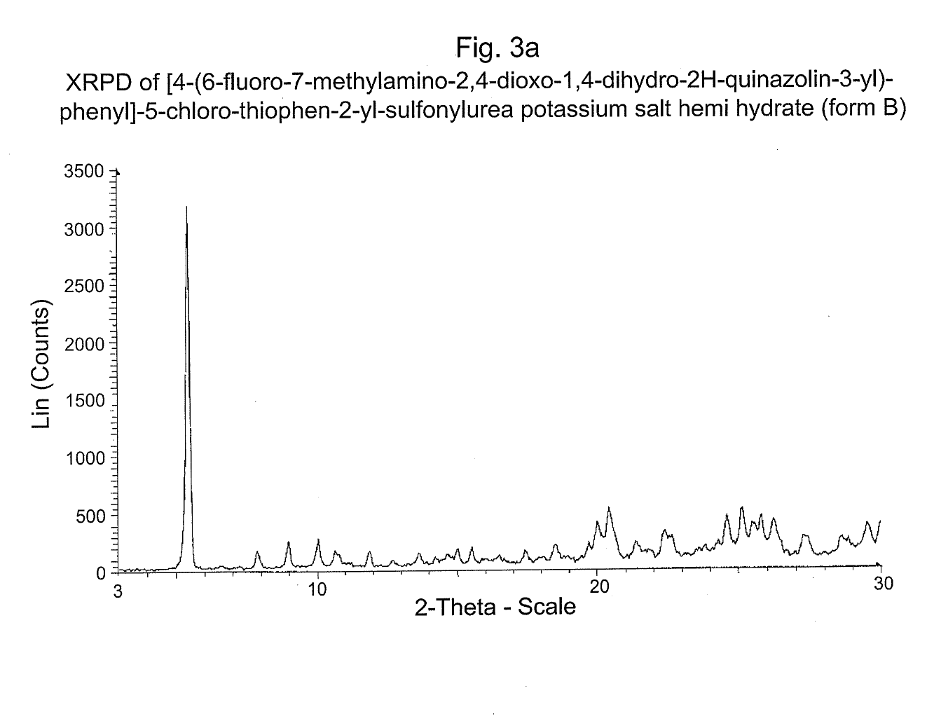

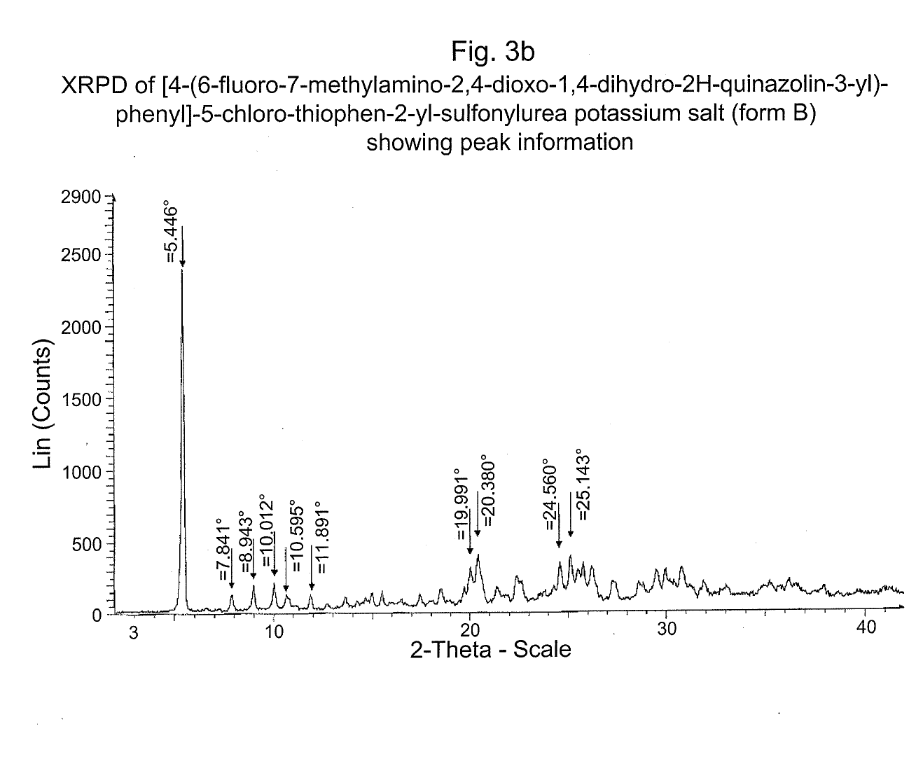

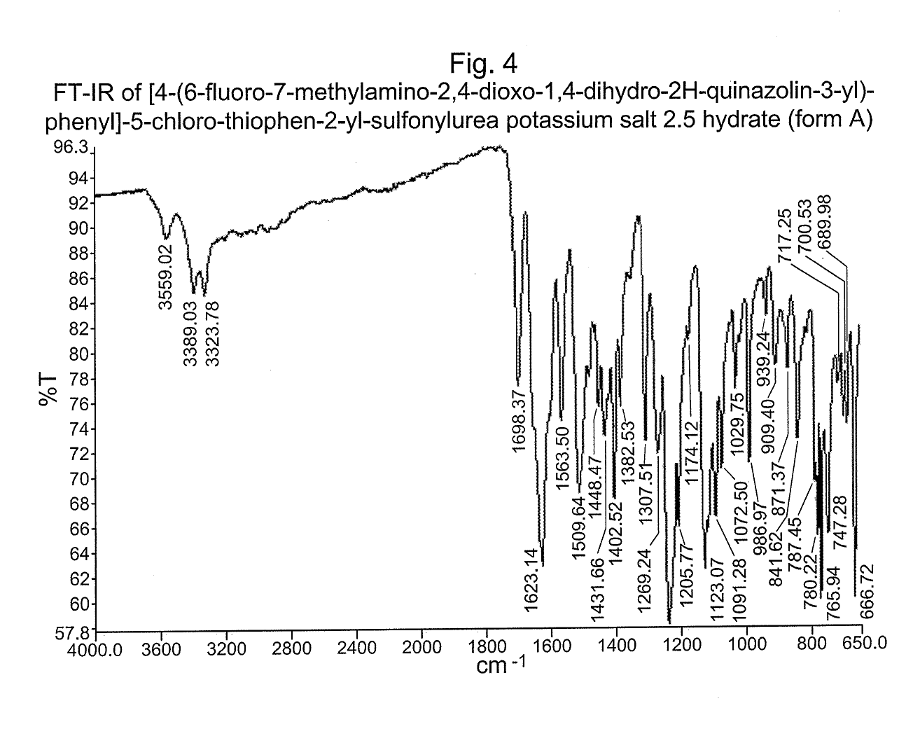

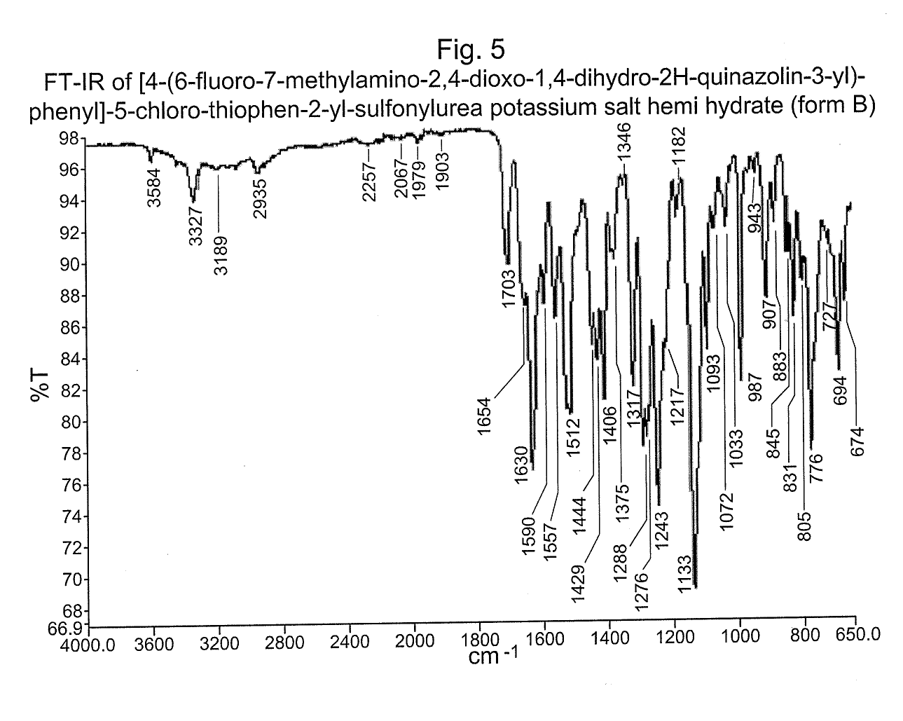

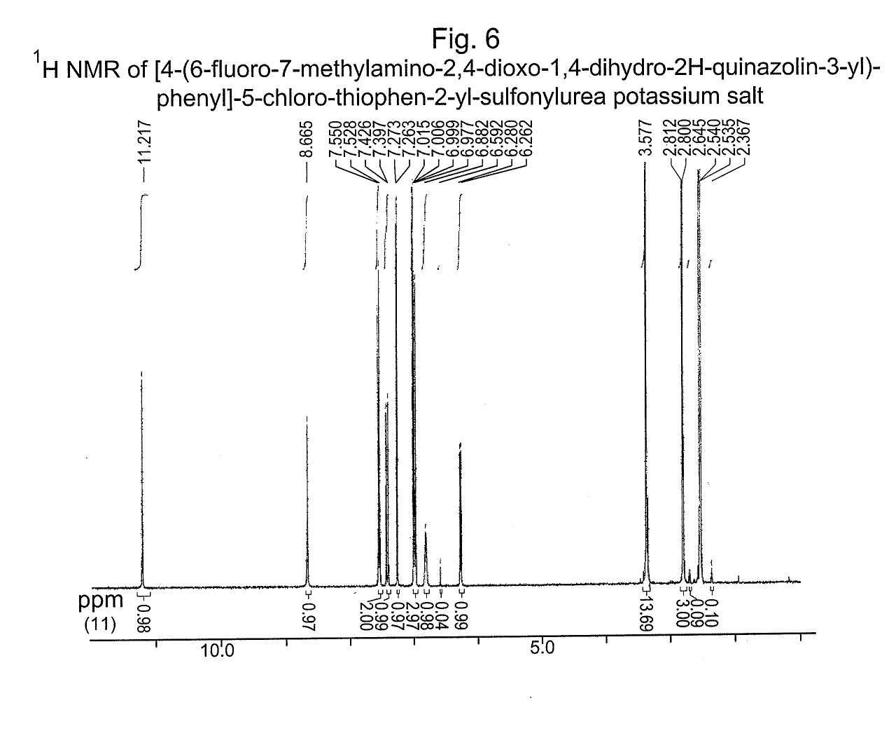

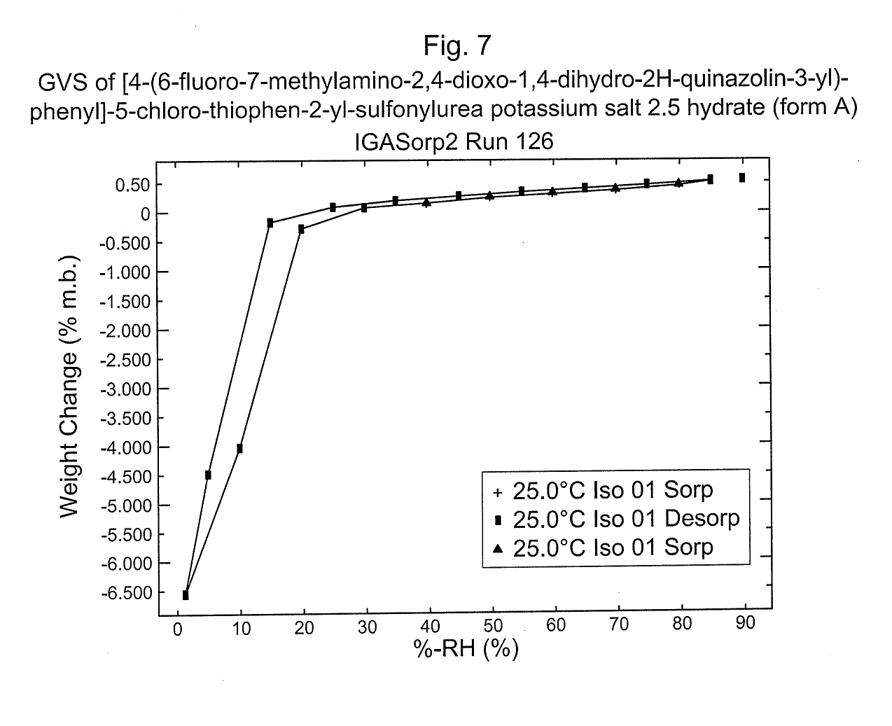

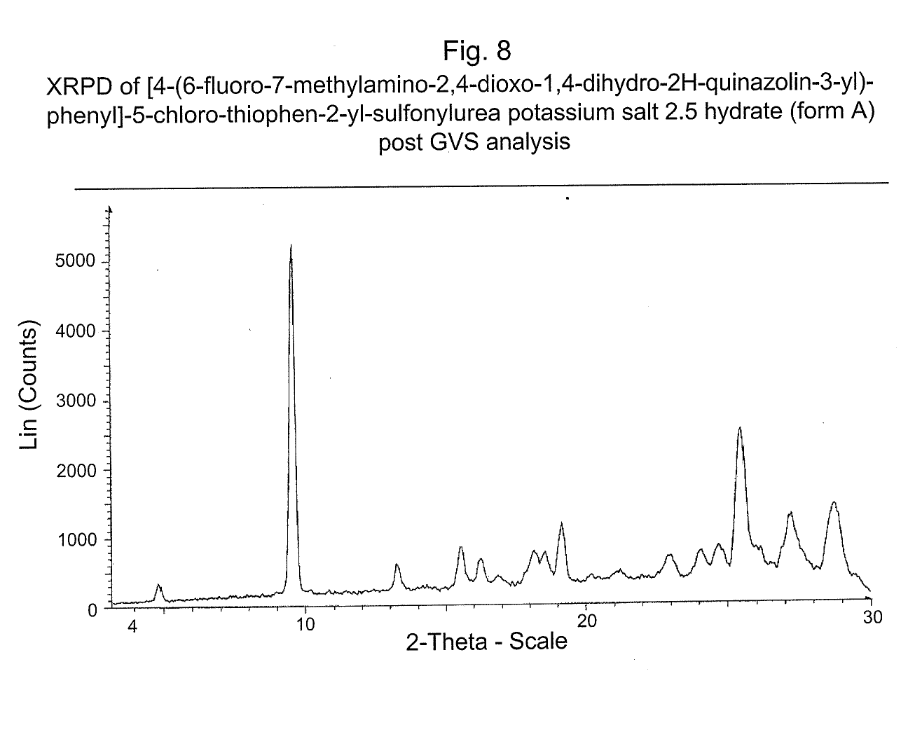

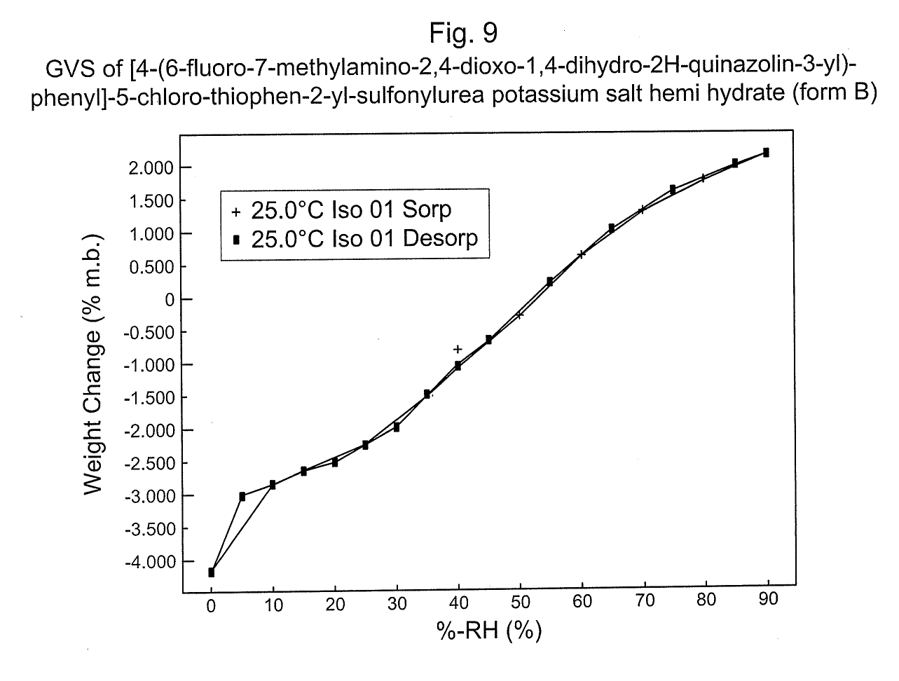

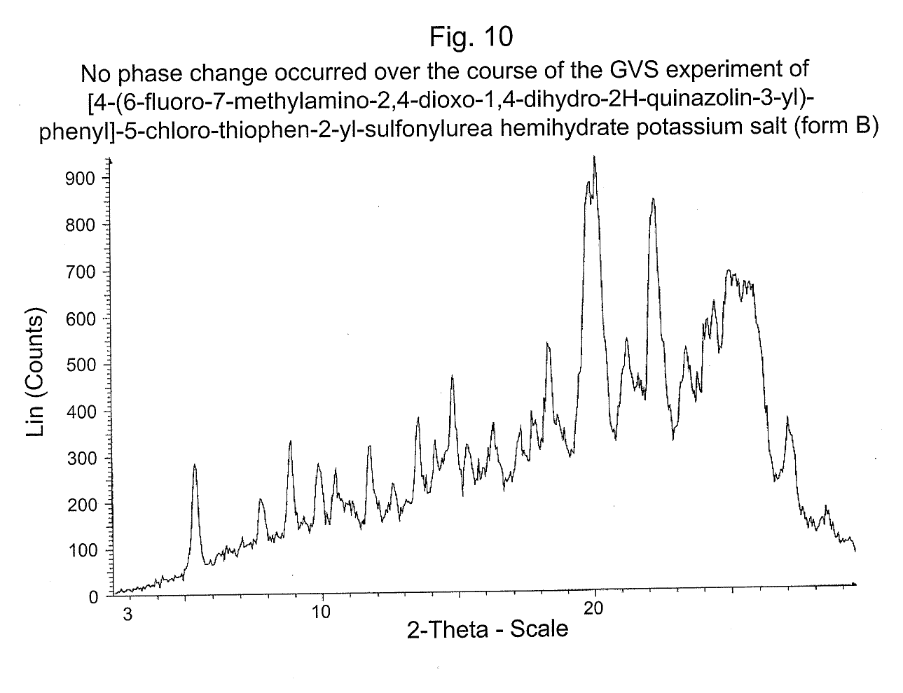

TABLE 2
Potassium salt form B XRPD Peak (°2θ) and % Intensity
Listing, data tabulated from FIG. 3b.

| Angle/2-Theta ° | Intensity | Intensity % |
|---|---|---|
| 5.45 | 2419.00 | 76.80 |
| 5.41 | 3152.00 | 100.00 |
| 7.84 | 176.00 | 5.60 |
| 8.94 | 242.00 | 7.70 |
| 10.01 | 261.00 | 8.30 |
| 10.60 | 152.00 | 4.80 |
| 11.89 | 162.00 | 5.10 |
| 19.99 | 409.00 | 13.00 |
| 20.38 | 532.00 | 16.90 |
| 24.56 | 452.00 | 14.30 |
| 25.14 | 523.00 | 16.60 |

Preferred orientation can affect peak intensities, and in some cases peak positions, in XRPD patterns. In the case of the potassium salts, preferred orientation has the most noticeable effect at lower angles. Preferred orientation causes some peaks in this region to be diminished (or increased). Crystal habit does not clearly differentiate between the solid forms; a variety of habits have been observed for each form, including needles, blades, plates, and irregular-shaped particles.

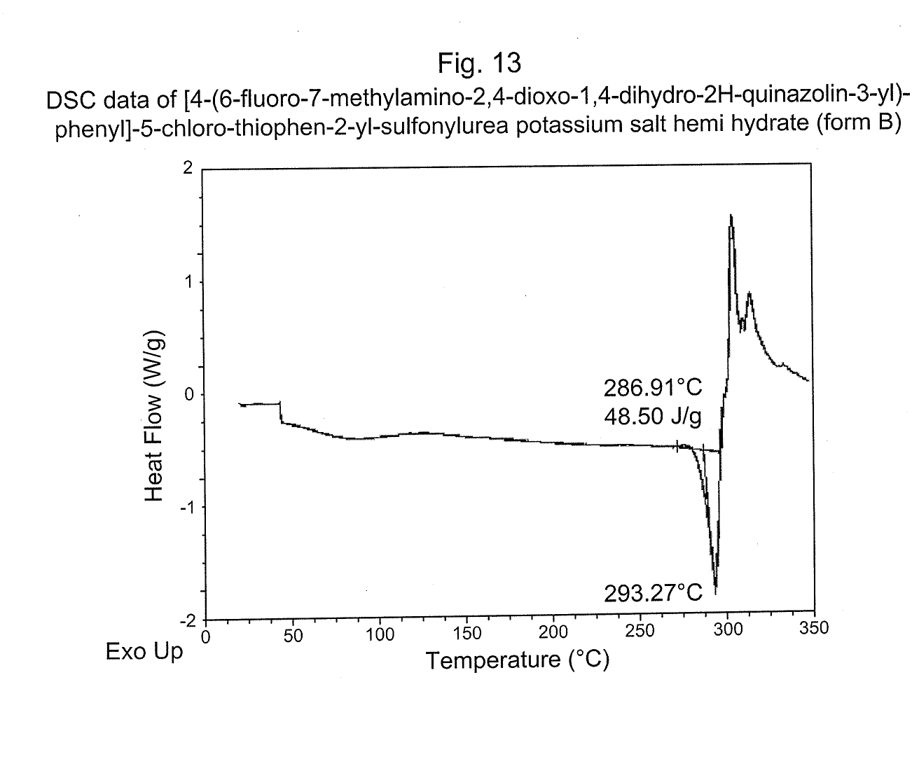

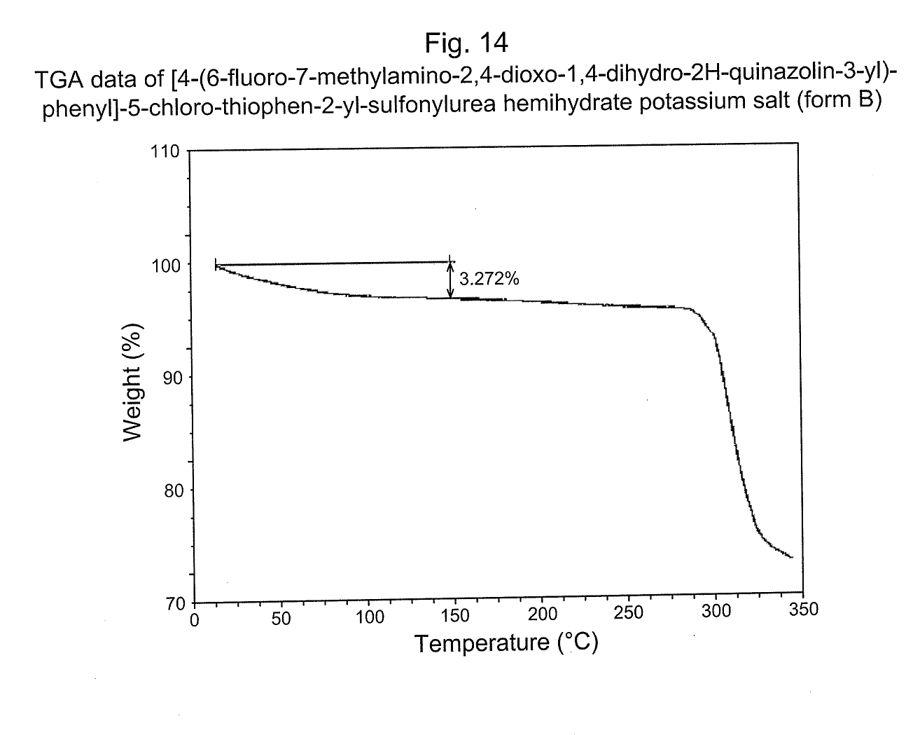

FIGS. 13 and 3 respectively show the DSC trace and the X-ray powder pattern for another crystalline solid, potassium salt form B. These results were observed when the remaining water was removed. In the DSC trace, an endotherm onset at about 287° C. is noteworthy, because the dehydrated form A melts at 238° C. The peaks at about 20.4 °2θ and 25.1 °2θ in the X-ray powder diffraction pattern also characterize form B with respect to form A, because form A does not have peaks to within 0.2° 2θ, the approximate precision of X-ray powder diffraction peaks, of the two characteristic form B peaks (see Tables 1 and 2). From that list, one sees that the peaks at about 20.4°2θ and 25.1°2θ (in Table 2 listed as 20.38 °2θ and 25.14 °2θ, respectively), when taken to one decimal point, is greater than 0.2° 2θ away from any peak in form A. Thus, the peaks at about 20.4 °2θ and 25.1 °2θ can be used to distinguish form B from form A.

### Potassium Salt Form C and D

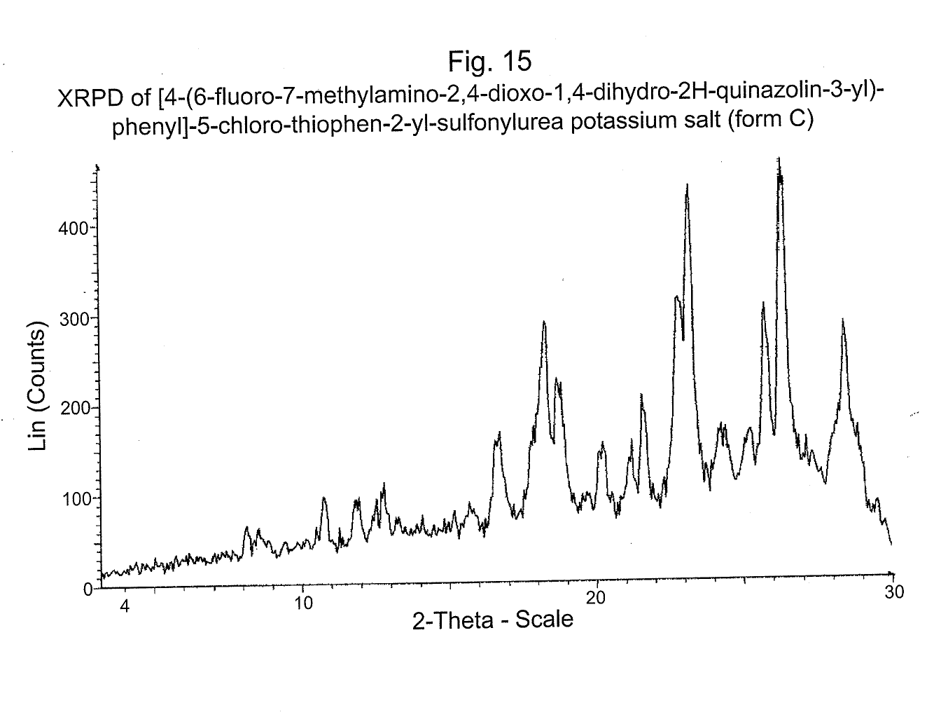

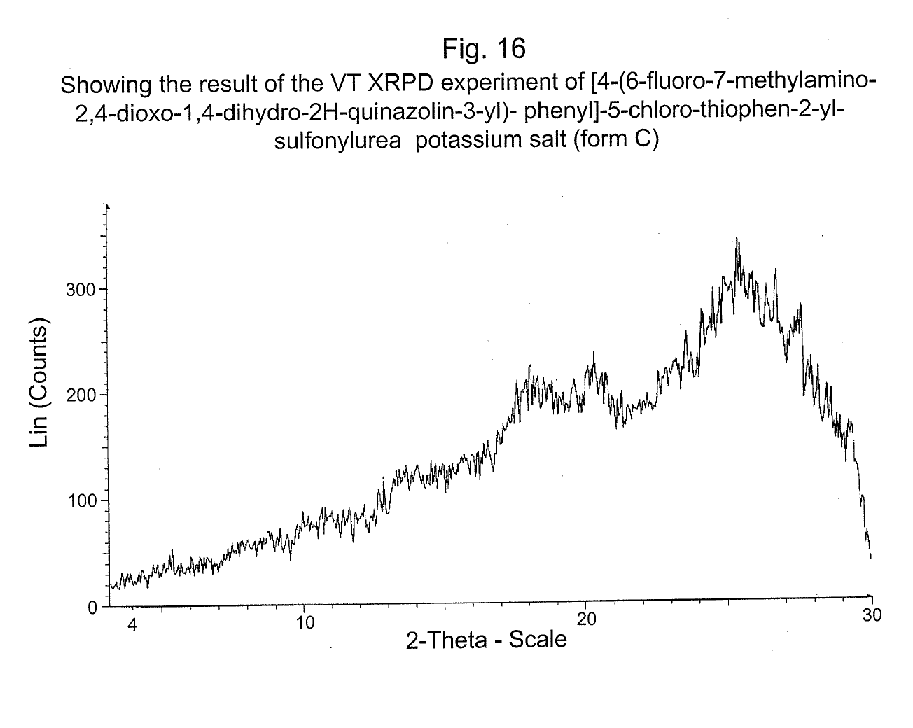

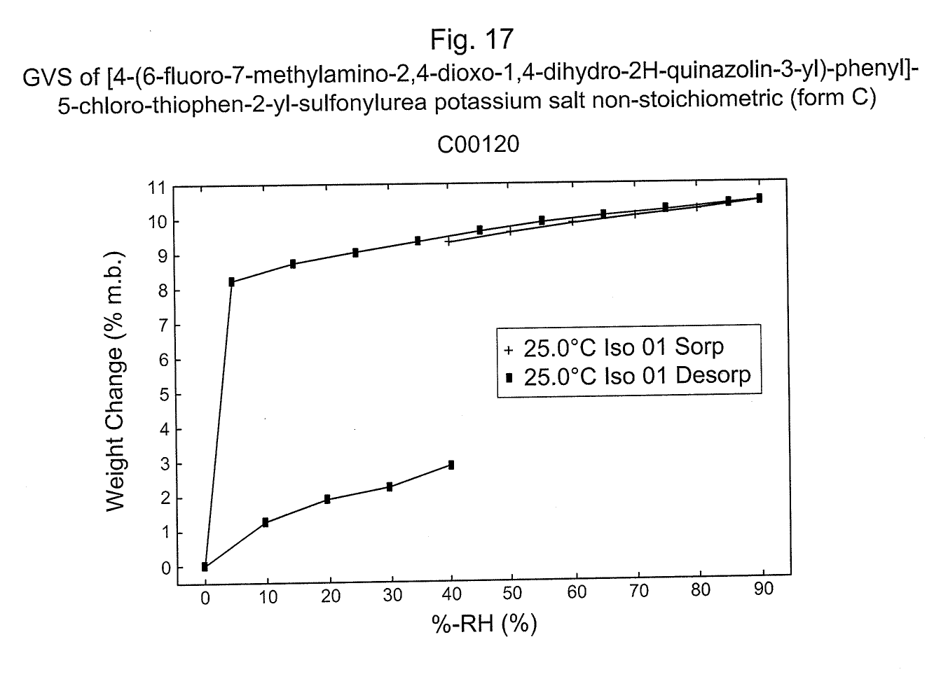

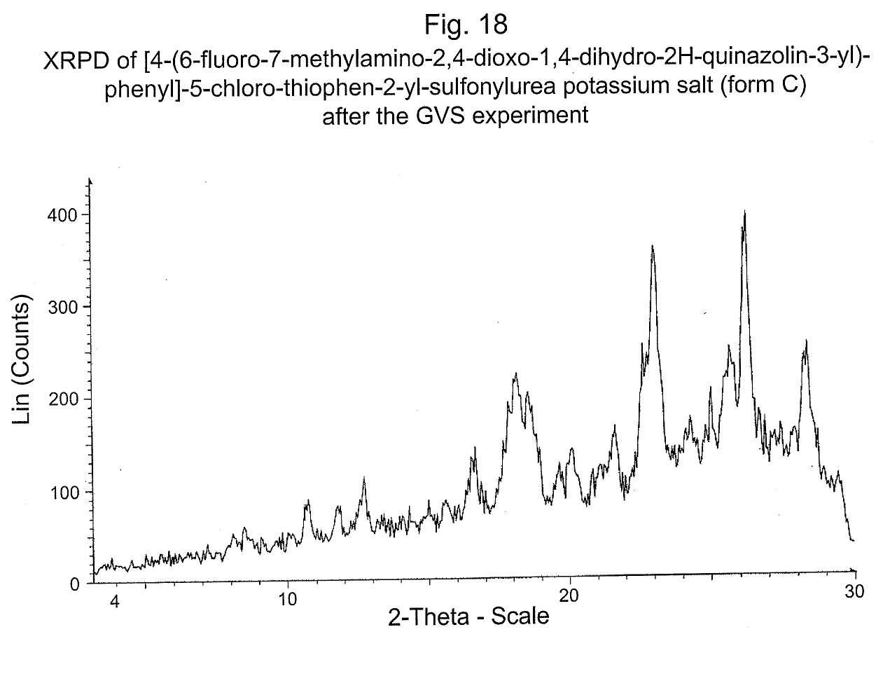

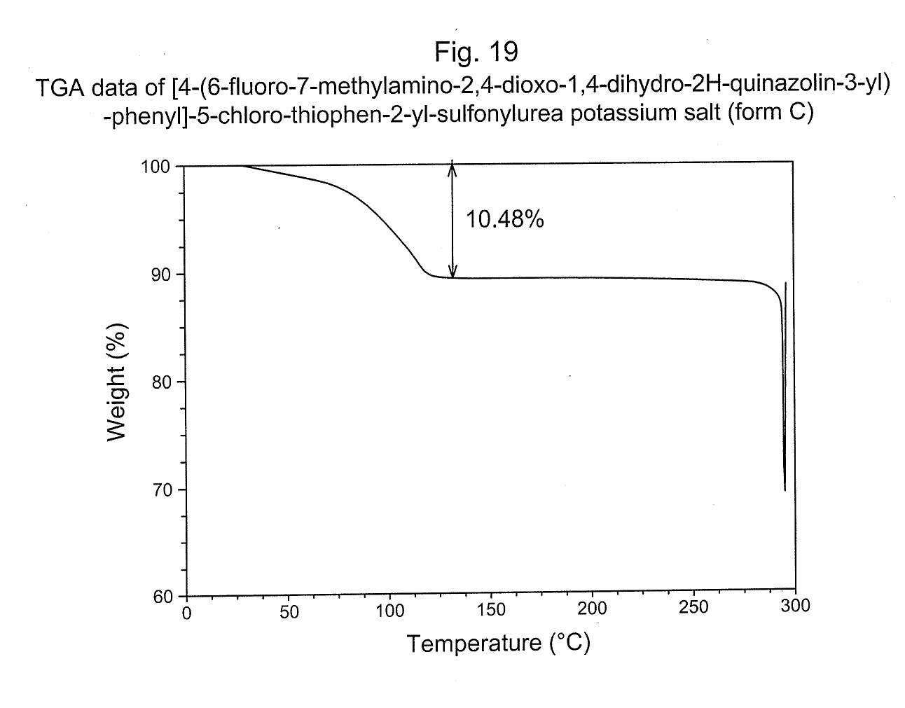

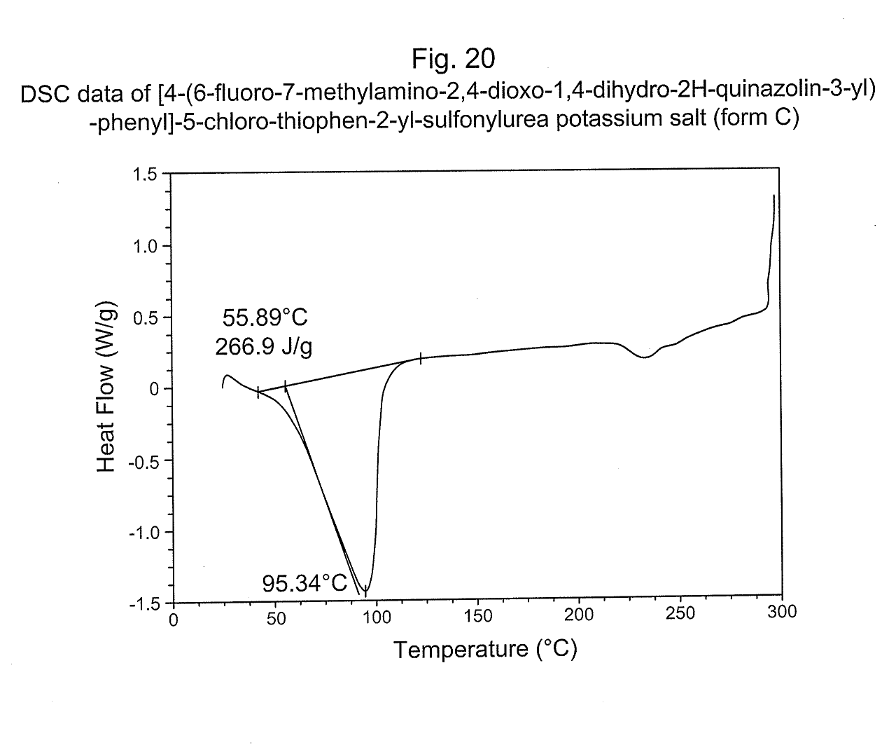

FIGS. 20 and 15 respectively show the DSC trace and the X-ray powder pattern for another crystalline solid form C. In the DSC trace, an endotherm onset at about 56° C. is noteworthy.

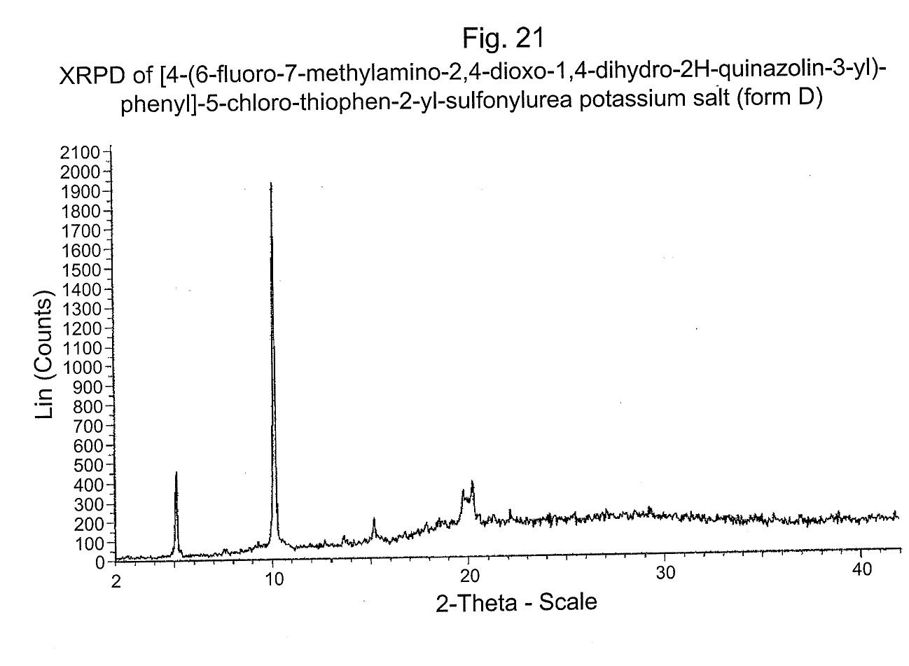

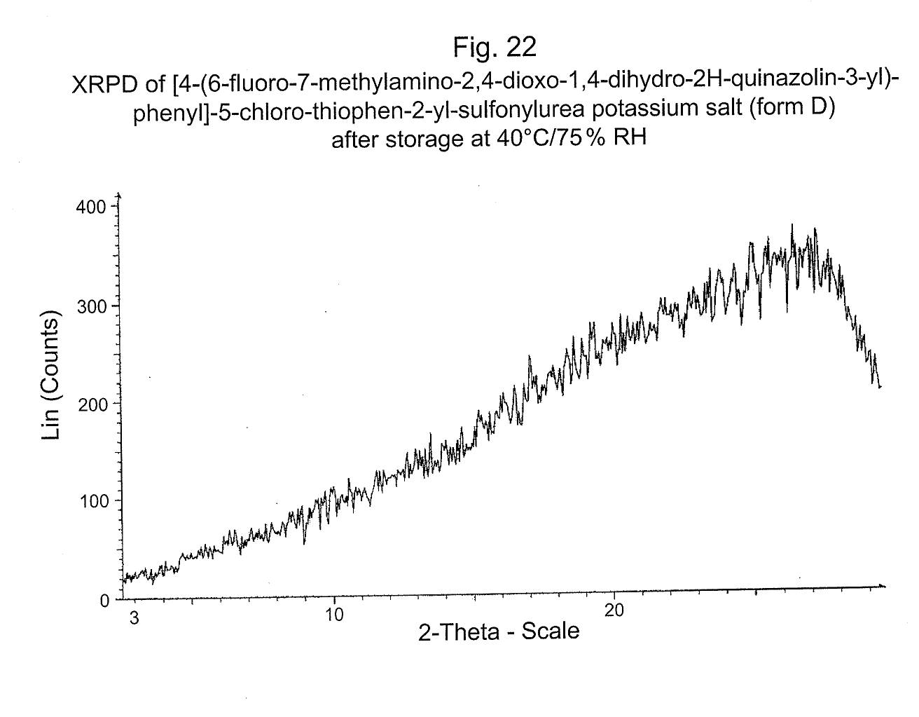

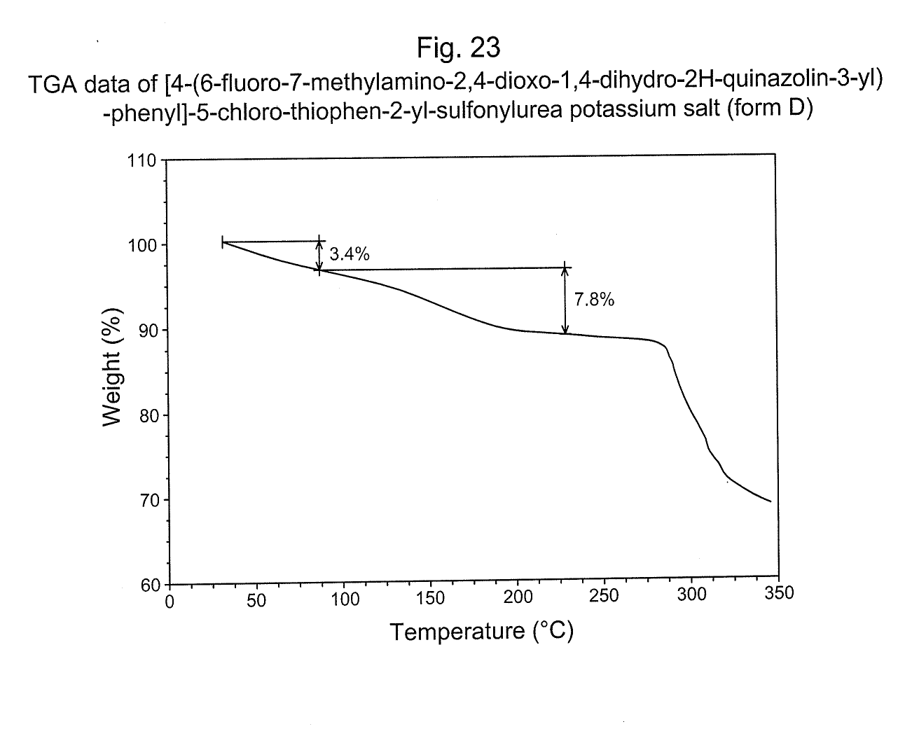

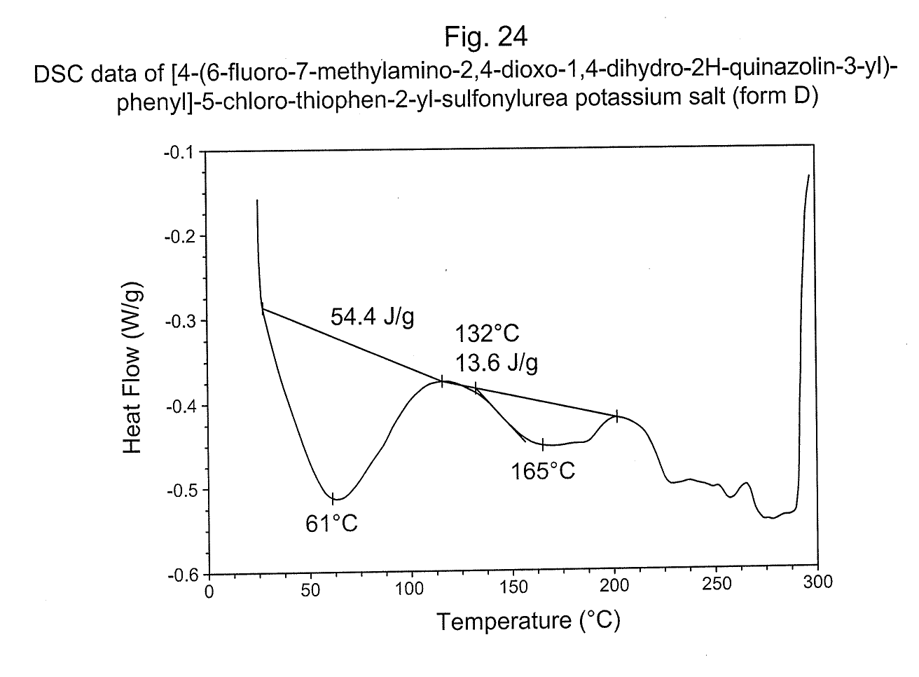

FIGS. 24 and 21 respectively show the DSC trace and the X-ray powder pattern for another crystalline solid form D. In the DSC trace, endotherms with onset temperatures at about 25° C. and 132° C. are noteworthy.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in new crystalline forms designated as form C and form D.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides at least one of:

(i) an X-ray powder diffraction pattern substantially in accordance with FIG. 21; and
(ii) a DSC scan substantially in accordance with FIG. 24; herein designated as form D.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides a DSC endotherm onset at about 56° C.; herein designated as form C.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides at least one of:

(i) an X-ray powder diffraction pattern substantially in accordance with FIG. 15; and
(ii) a DSC scan substantially in accordance with FIG. 20; herein designated as form C.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form, including a substantially pure form, which provides DSC endotherms with onset temperatures at about 25° C. and 132° C.; herein designated as form D.

In another embodiment the present invention provides [4-(6-fluoro-7-methylamino-2,4-dioxo-1,4-dihydro-2H-quinazolin-3-yl)-phenyl]-5-chloro-thiophen-2-yl-sulfonylurea potassium salt in an amorphous form.

### Sodium Salt Forms A, B and C

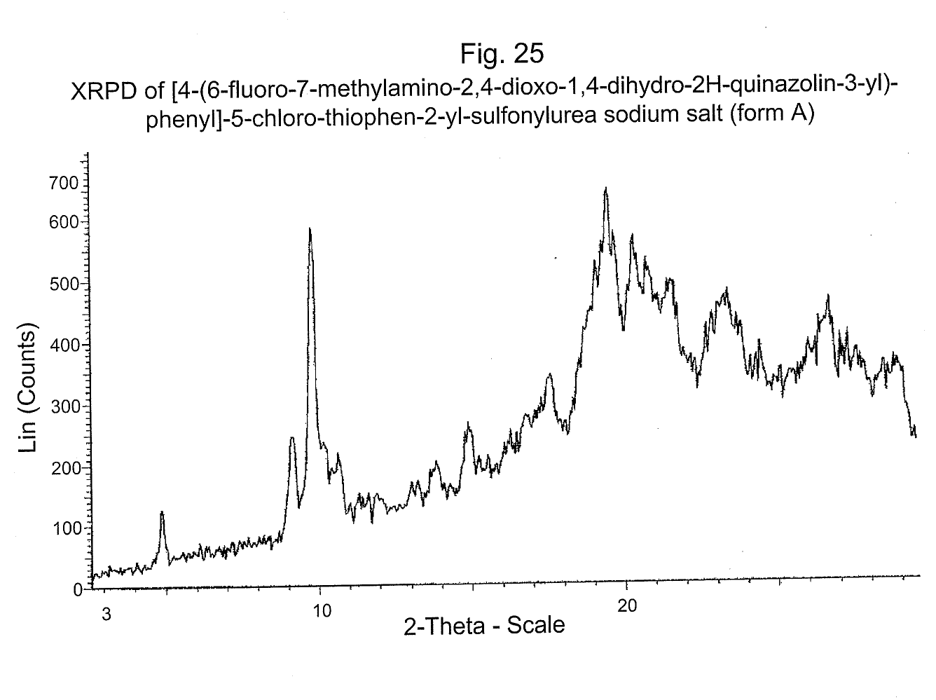

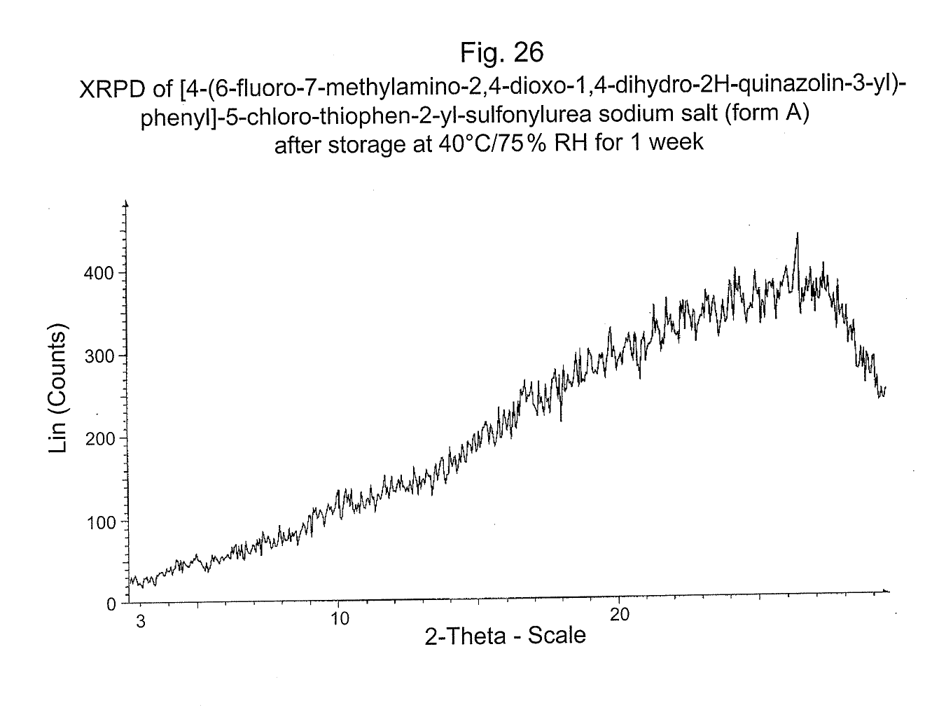

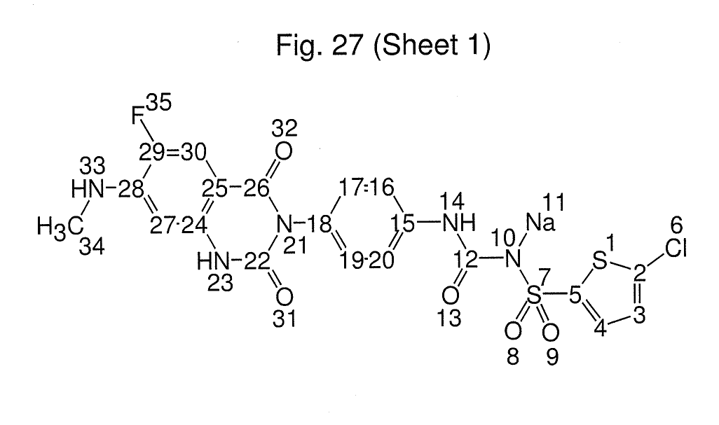

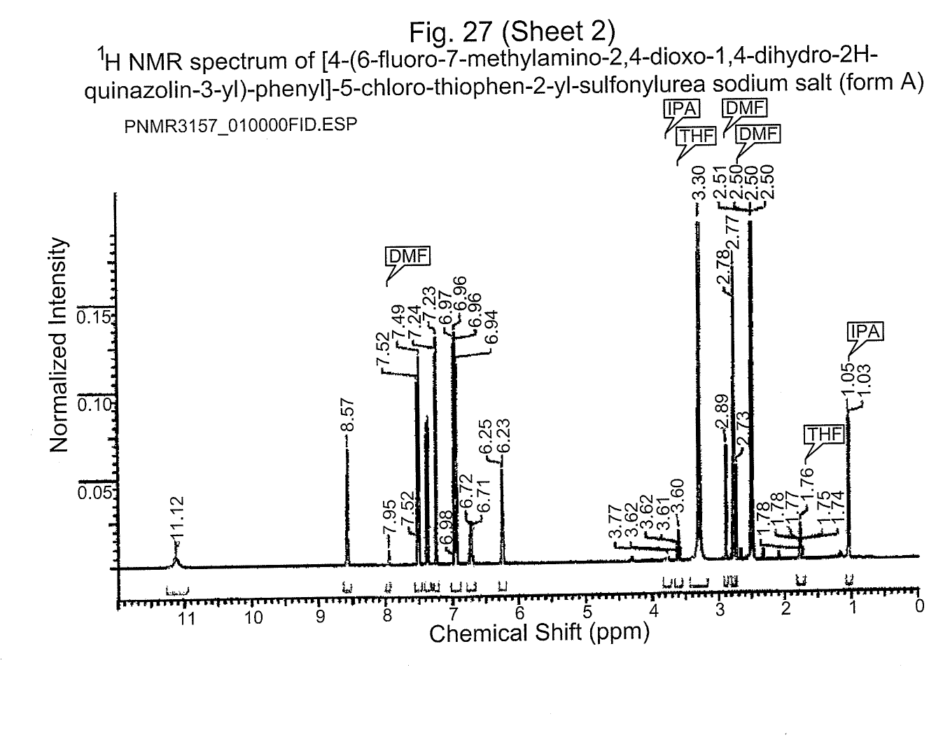

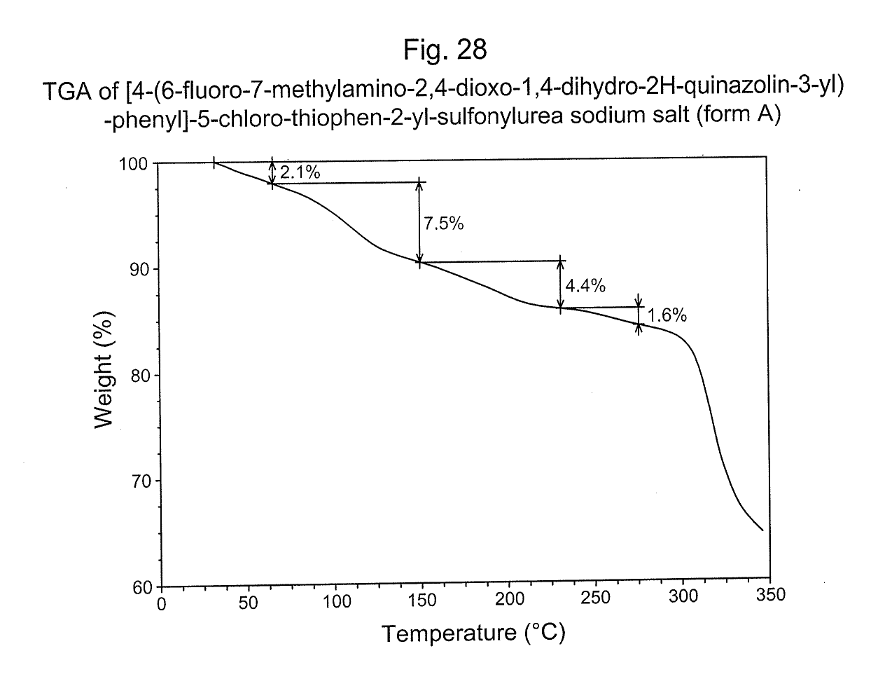

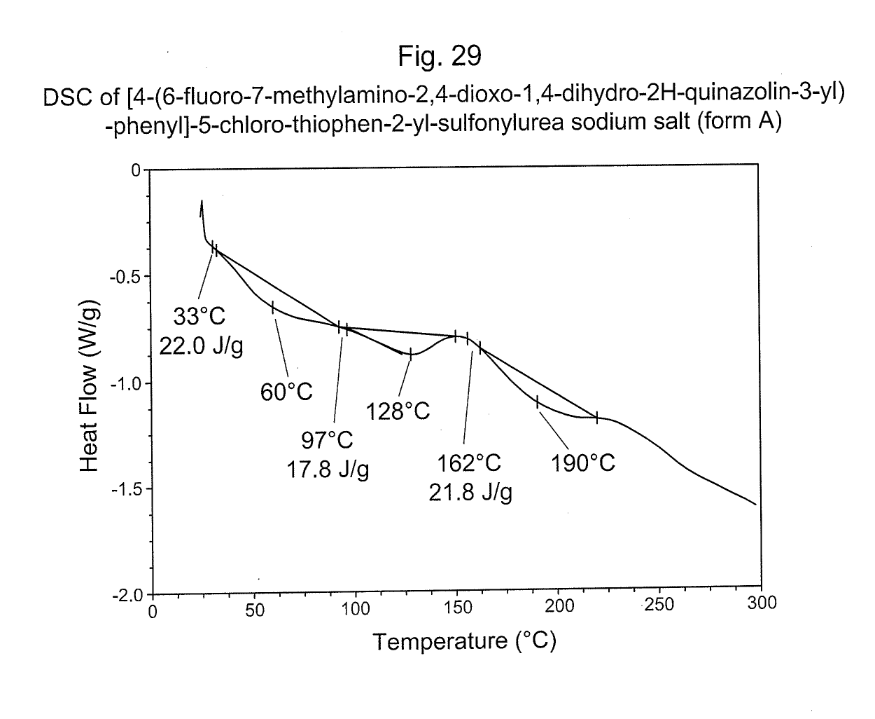

FIGS. 29 and 25 respectively show the DSC trace and the X-ray powder pattern for another crystalline solid form A. In the DSC trace, multiple endotherms with onset temperatures at about 33° C., 97° C. and 162° C. are noteworthy.

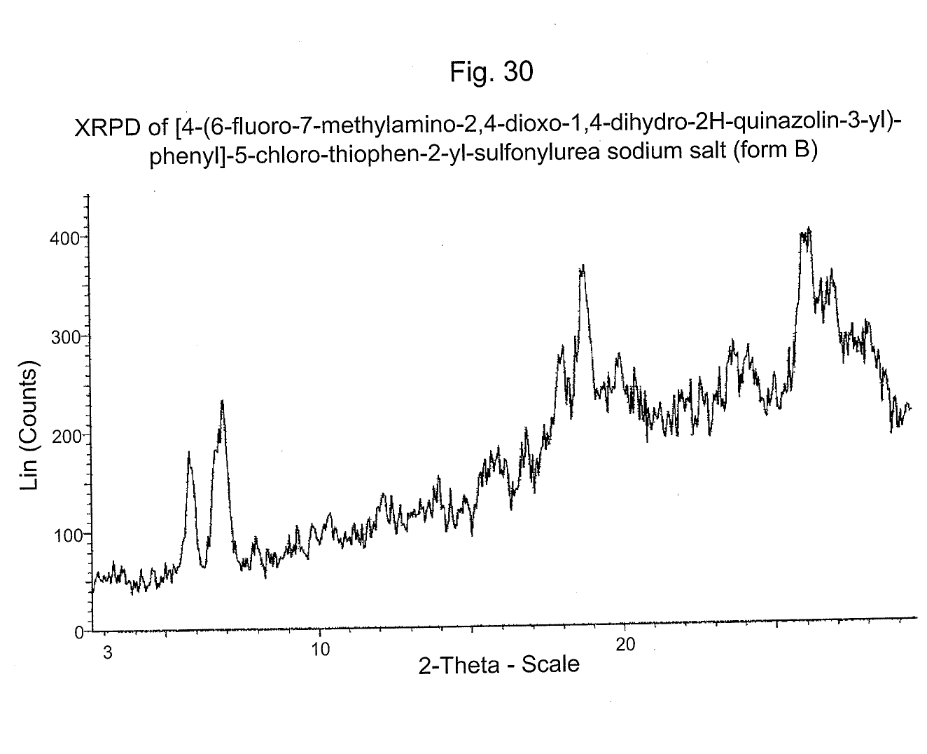

FIG. 30 shows the X-ray powder pattern for another crystalline solid form B.

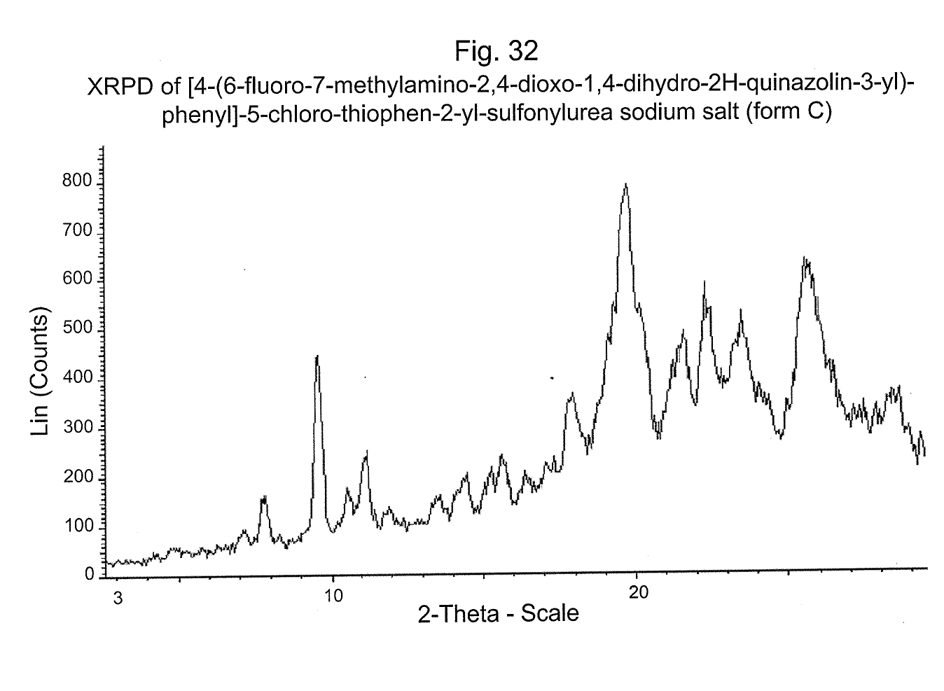

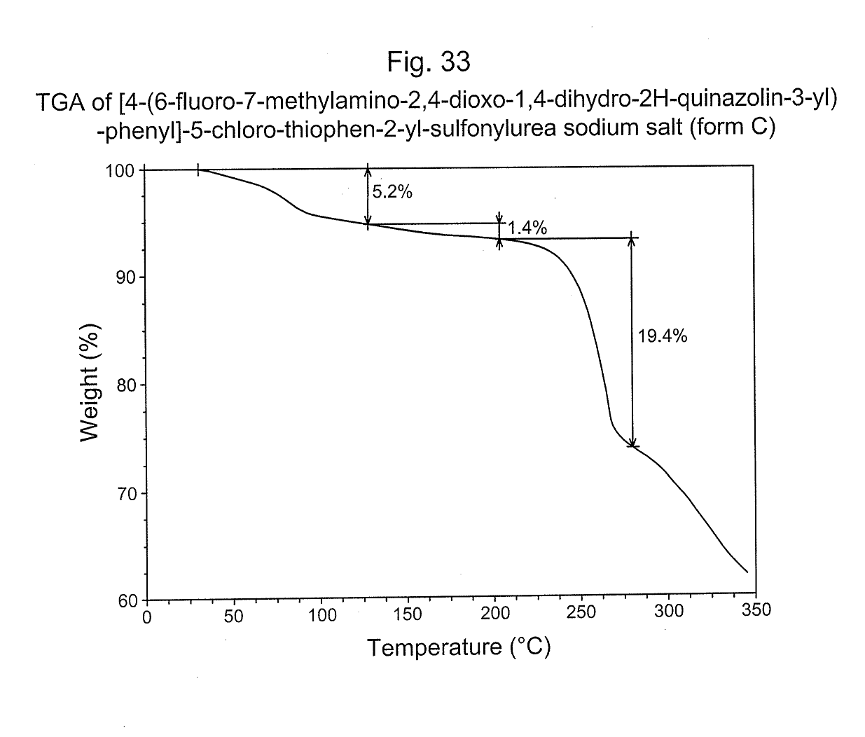

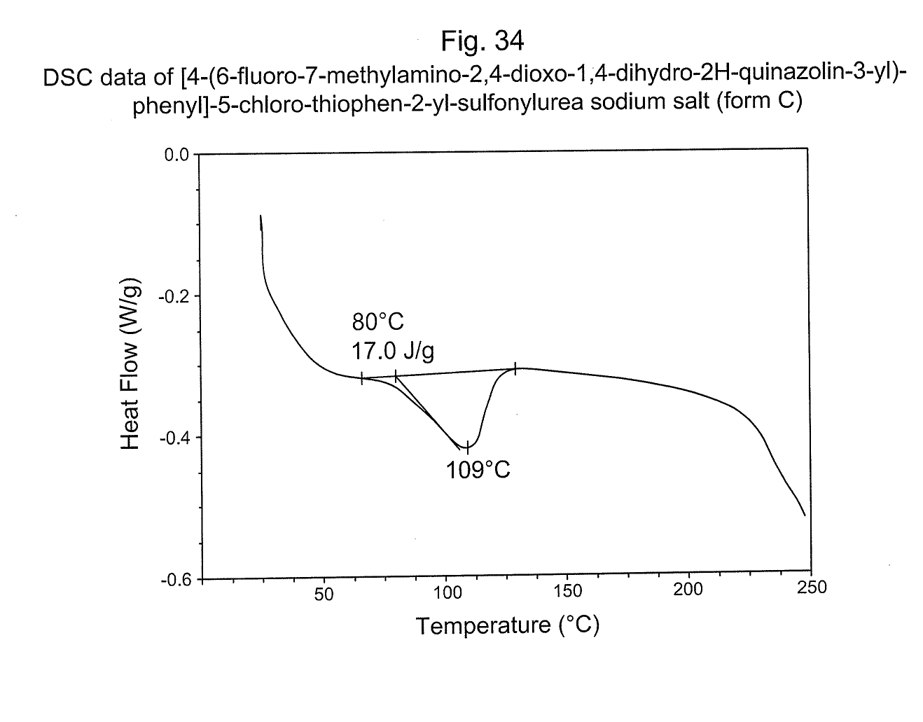

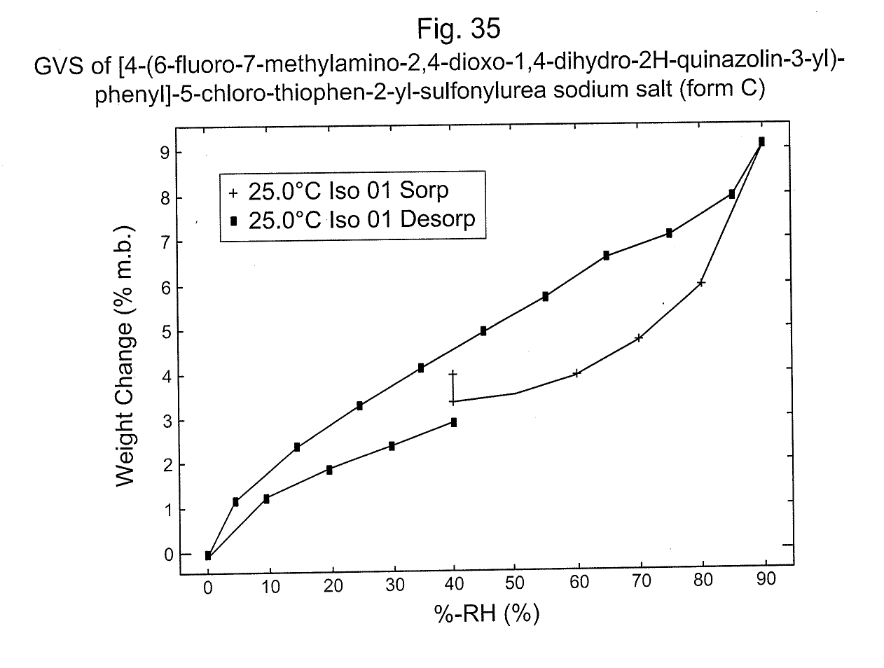

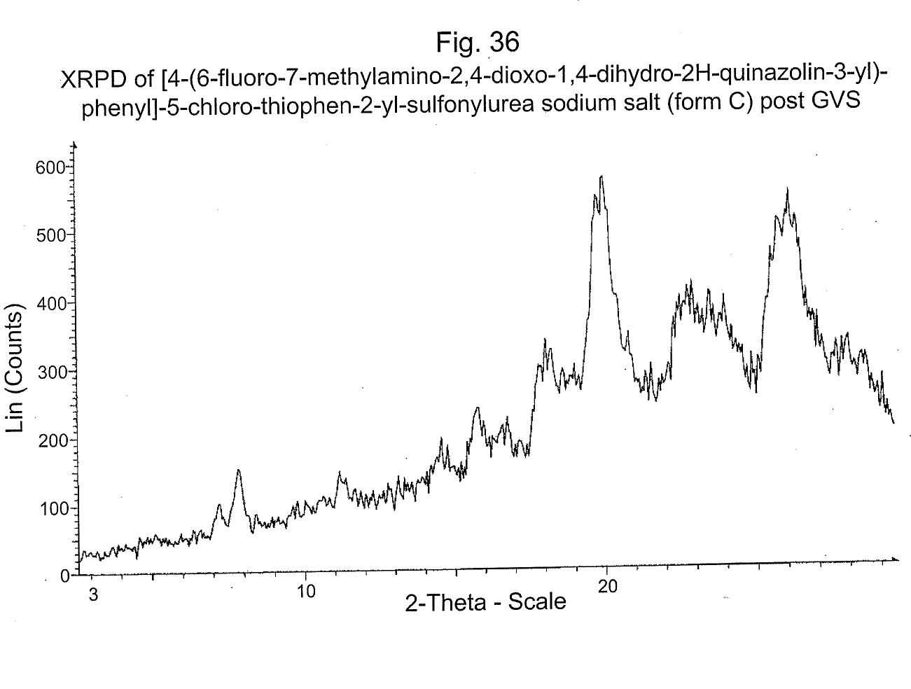

FIGS. 34 and 32 respectively show the DSC trace and the X-ray powder pattern for another crystalline solid form C. In the DSC trace an endotherm onset at about 80° C. is noteworthy.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in new crystalline forms designated as form A, form B and form C.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form, including a substantially pure form, which provides at least one of:

(i) an X-ray powder diffraction pattern substantially in accordance with FIG. 25; and
(ii) a DSC scan substantially in accordance with FIG. 29; herein designated as form A.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form, including a substantially pure form, which provides a DSC trace with endotherm onset temperatures at about 33° C., 97° C. and 162° C.; herein designated as form A.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form, including a substantially pure form, which provides:

(i) an X-ray powder diffraction pattern substantially in accordance with FIG. 30; herein designated as form B.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form, including a substantially pure form, which provides at least one of:

(i) an X-ray powder diffraction pattern substantially in accordance with FIG. 32; and
(ii) a DSC scan substantially in accordance with FIG. 34; herein designated as form C.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form, including a substantially pure form, which provides a DSC endotherm onset at about 80° C.; herein designated as form C.

### Calcium Salt Form A

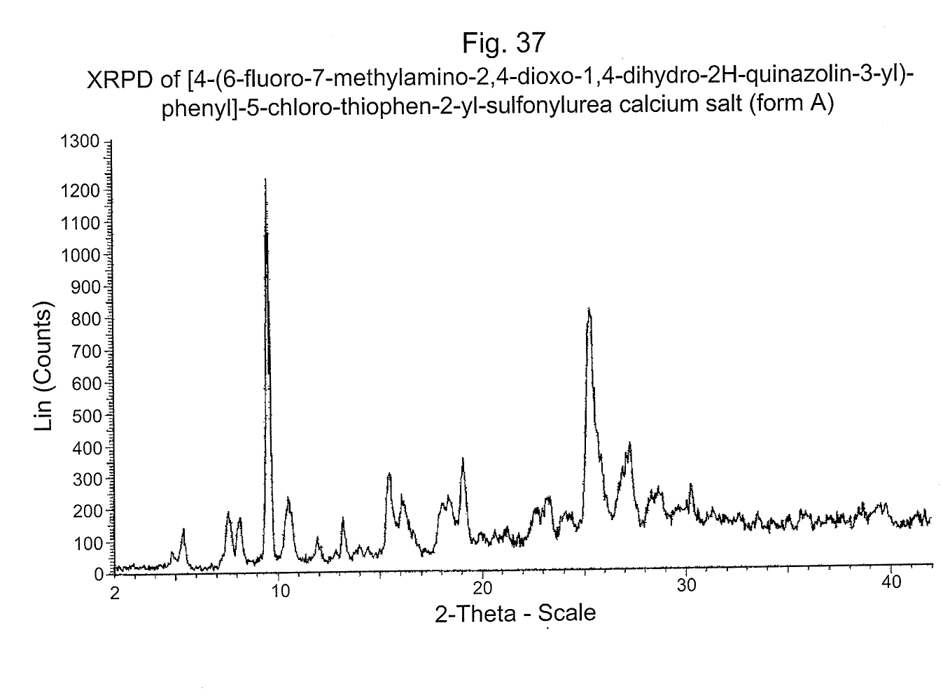

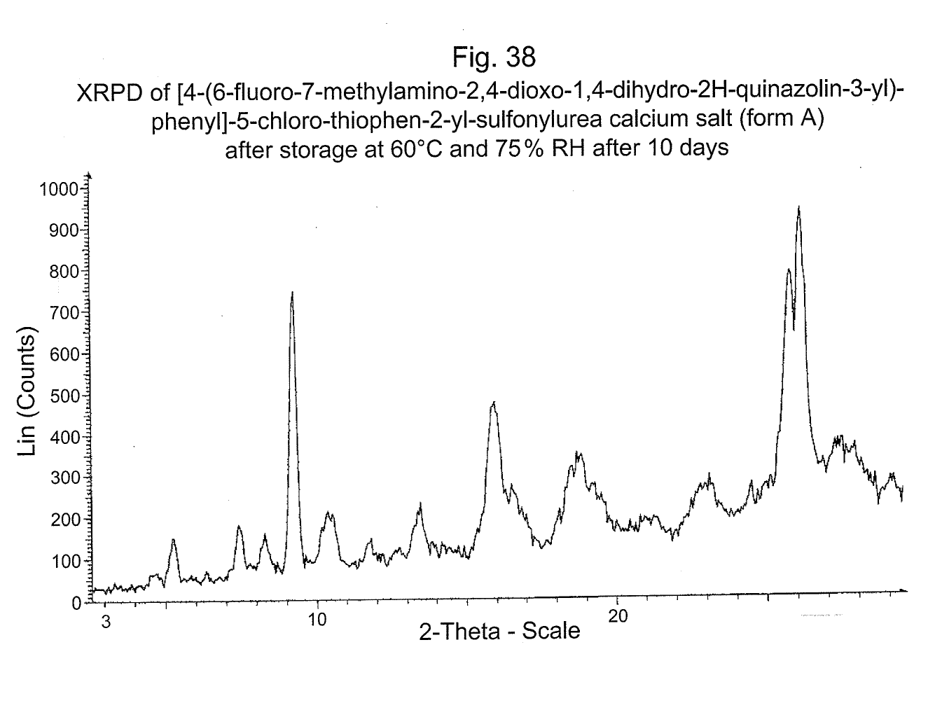

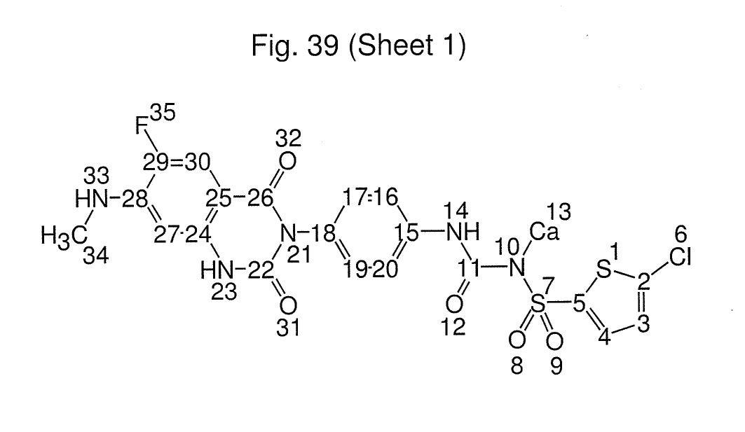

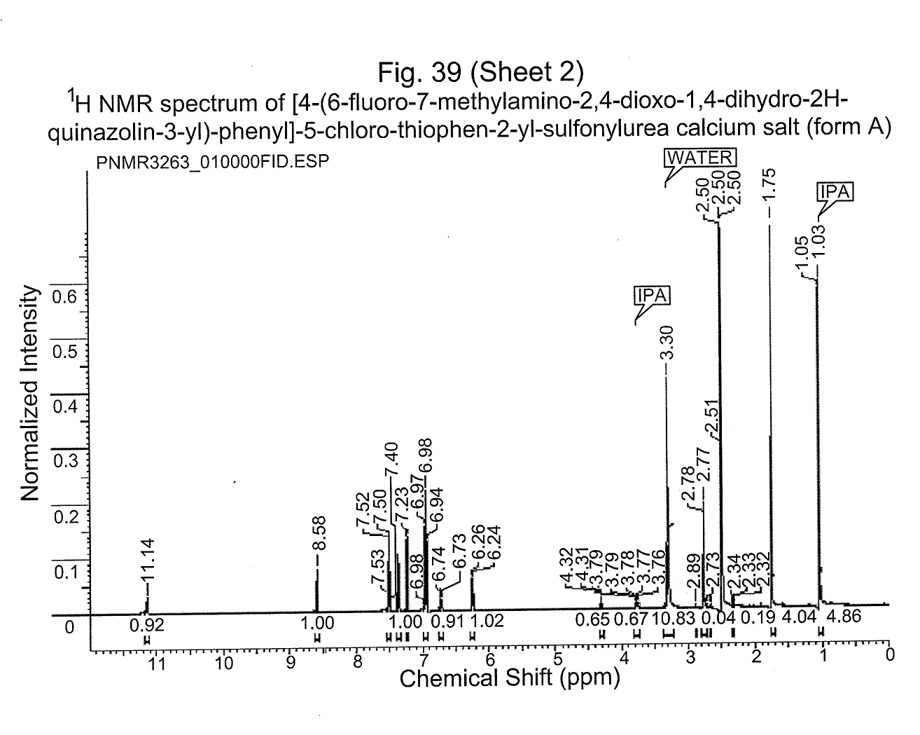

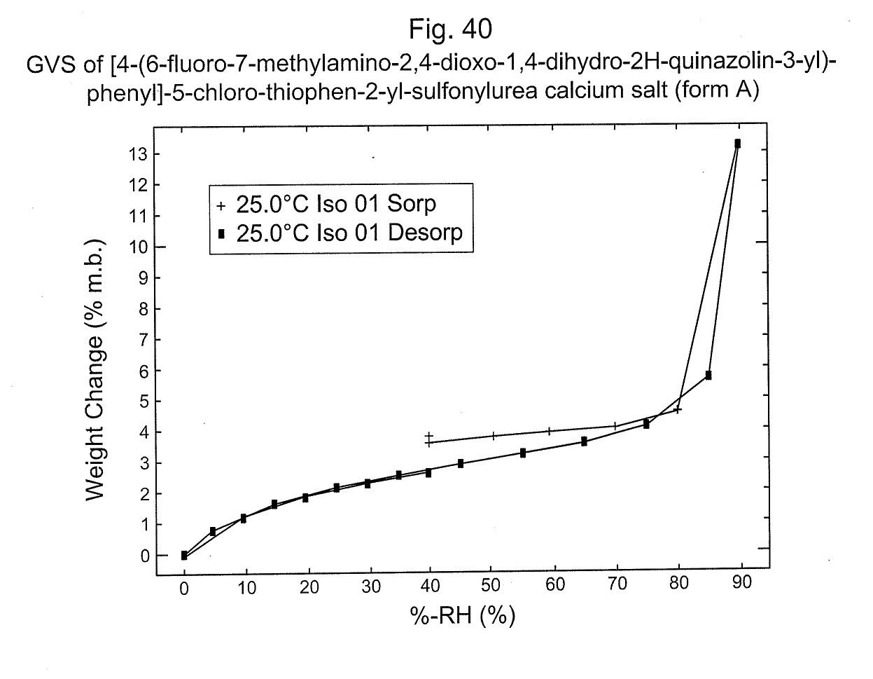

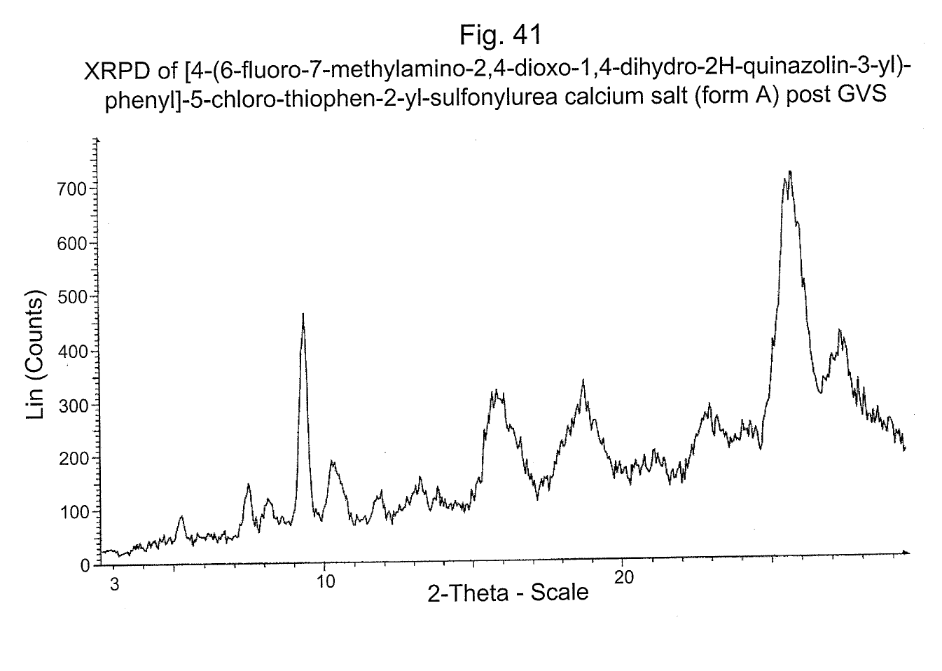

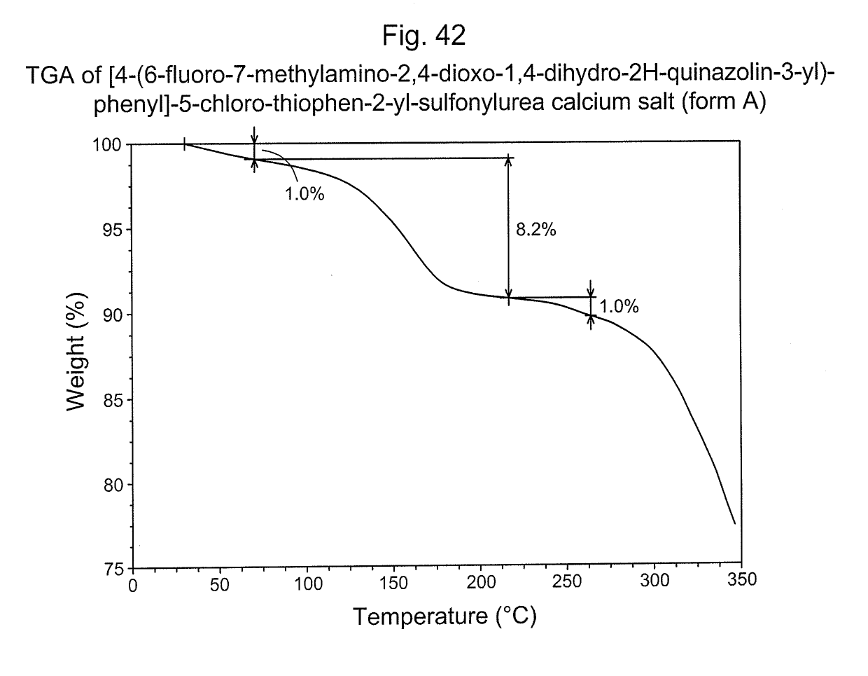

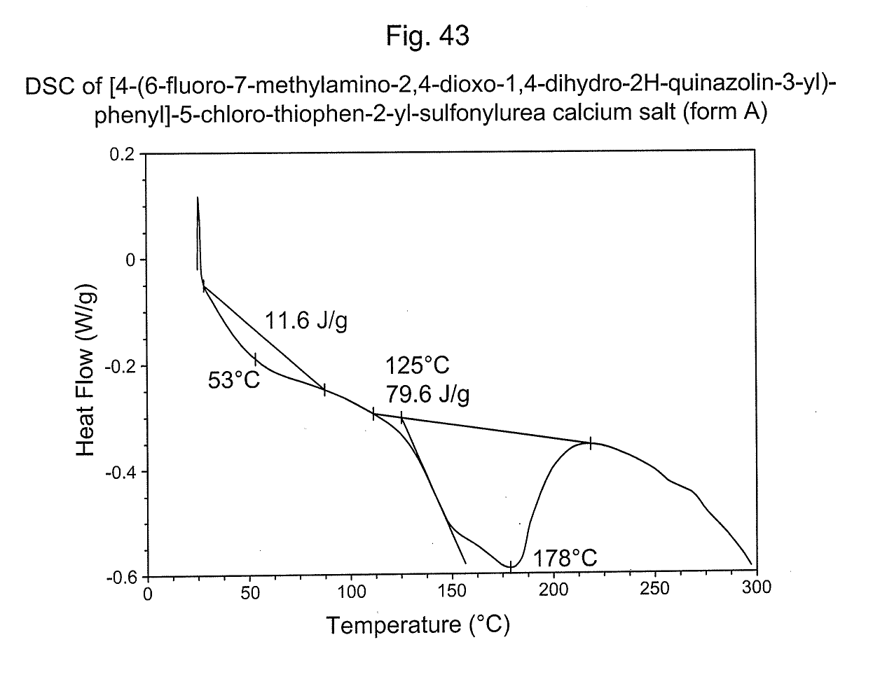

FIGS. 43 and 37 respectively show the DSC trace and the X-ray powder pattern for another crystalline solid form A. In the DSC trace, an endotherm onset at about 125° C. is noteworthy.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea calcium salt in a new crystalline form designated as form A.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea calcium salt in a crystalline solid form, including a substantially pure form, which provides at least one of:

(i) an X-ray powder diffraction pattern substantially in accordance with FIG. 37; and
(ii) a DSC scan substantially in accordance with FIG. 43; herein designated as form A.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea calcium salt in a crystalline solid form, including a substantially pure form, which provides a DSC endotherm onset at about 125° C.; herein designated as form A.

### Tromethamine Salt Form A

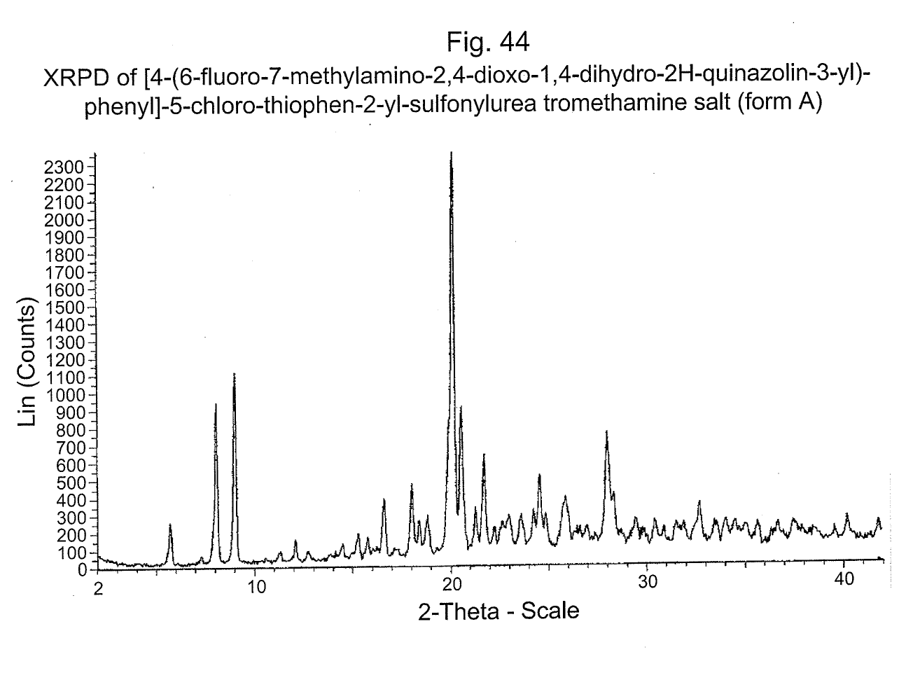

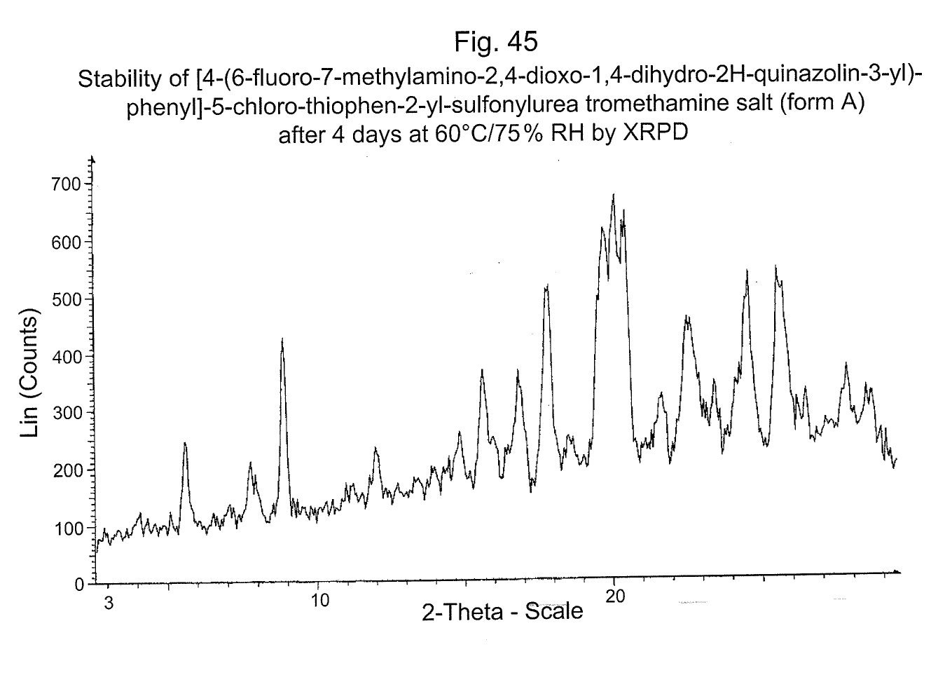

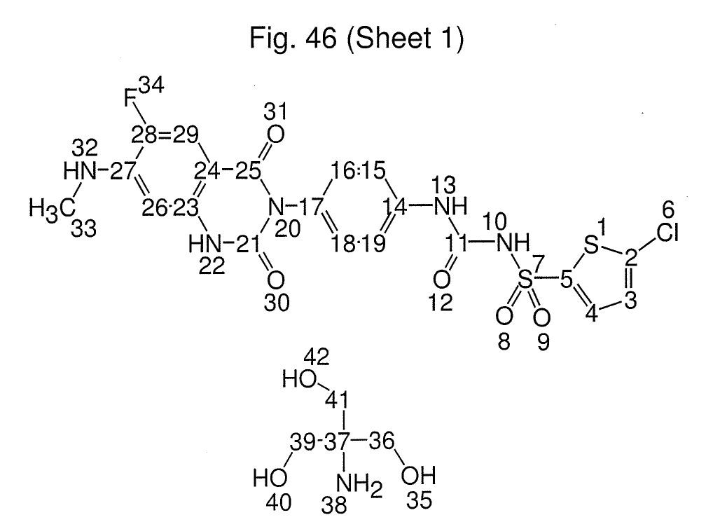

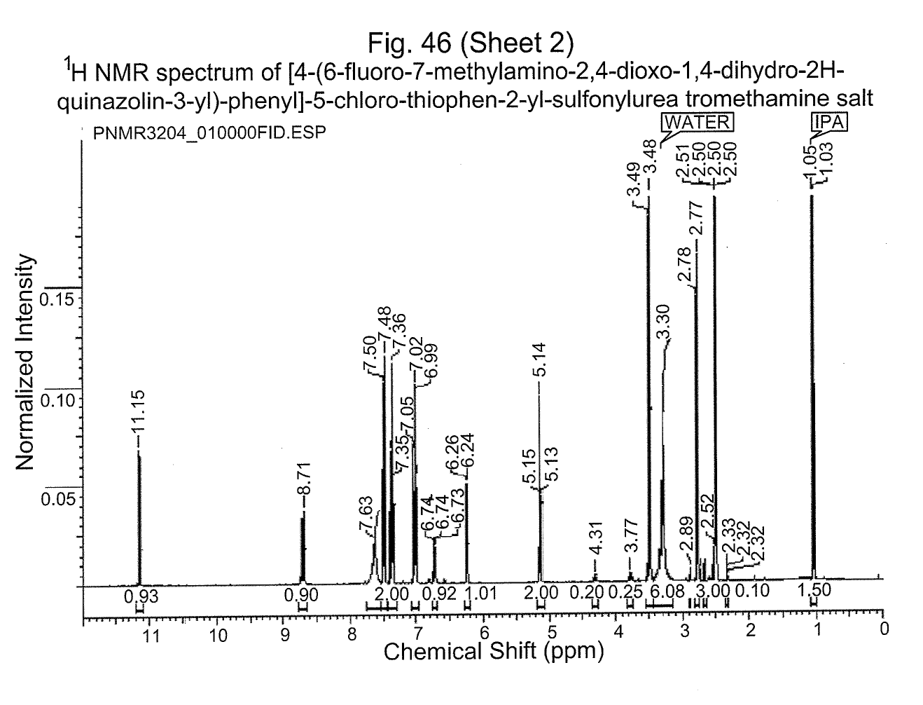

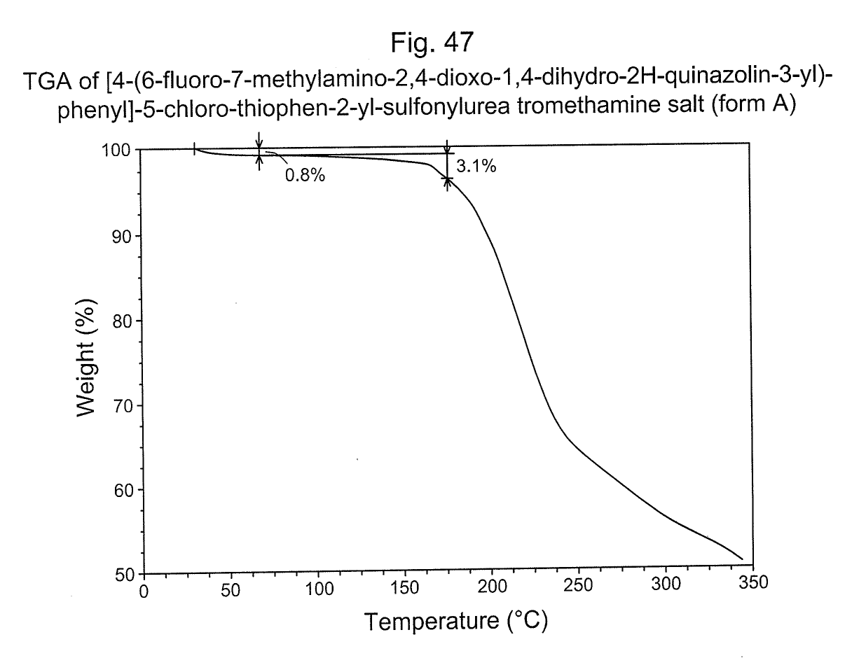

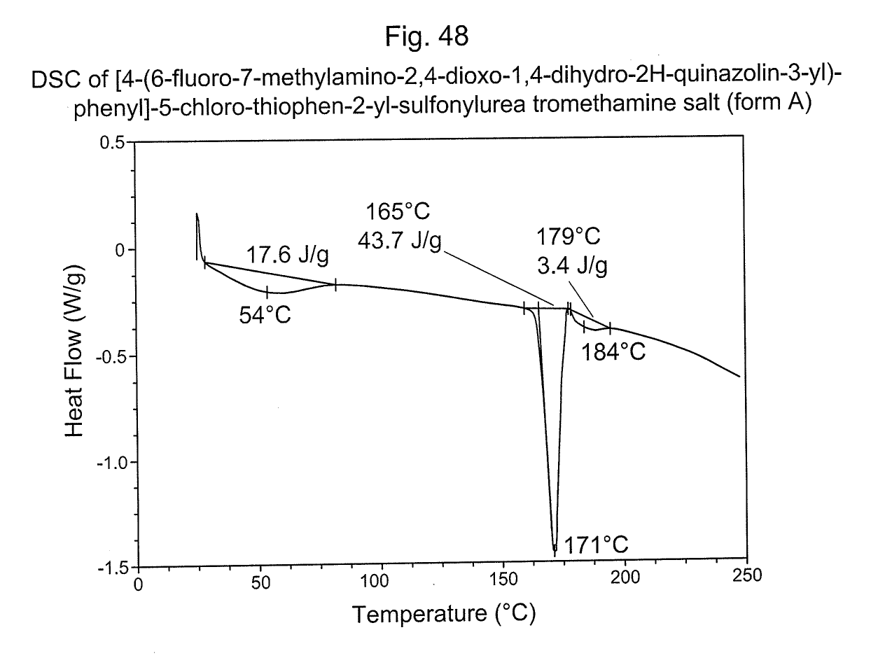

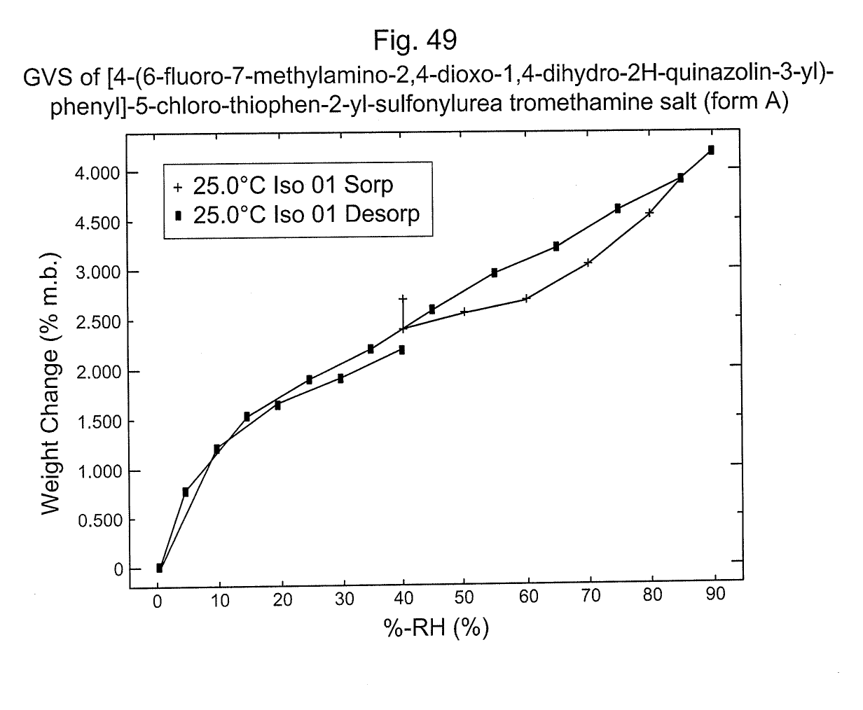

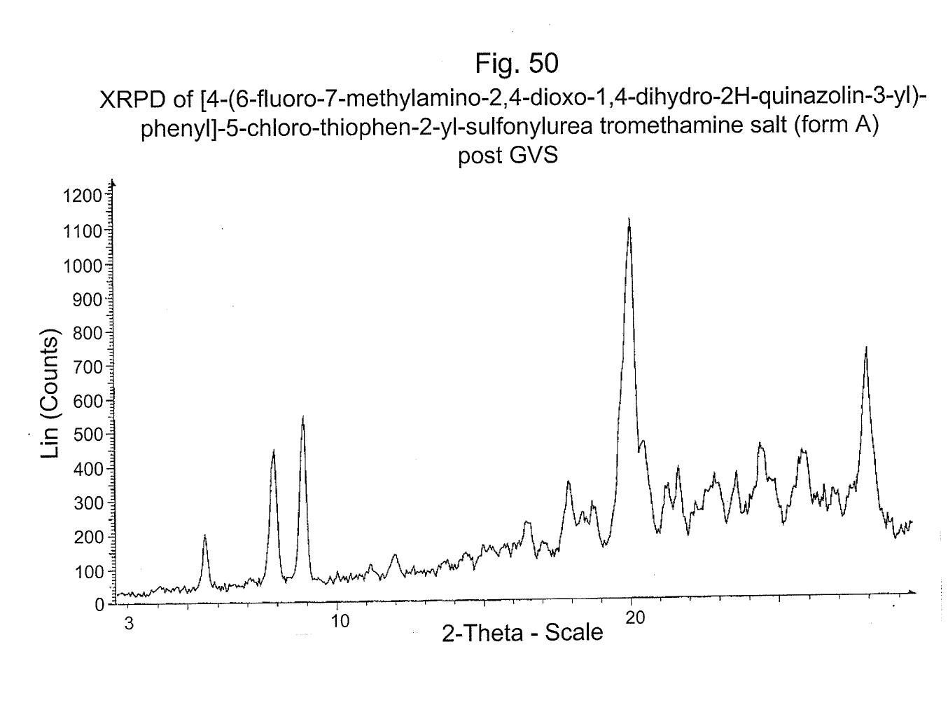

FIGS. 48 and 44 respectively show the DSC trace and the X-ray powder pattern for another crystalline solid form A. In the DSC trace, an endotherm onset at about 165° C. is noteworthy.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea tromethamine salt in a new crystalline form designated as form A.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea tromethamine salt in a crystalline solid form, including a substantially pure form, which provides at least one of:

(i) an X-ray powder diffraction pattern substantially in accordance with FIG. 44; and
(ii) a DSC scan substantially in accordance with FIG. 48; herein designated as form A.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea tromethamine salt in a crystalline solid form, including a substantially pure form, which provides a DSC endotherm onset at about 165° C.; herein designated as form A.

### Ammonium Salt Forms A and B

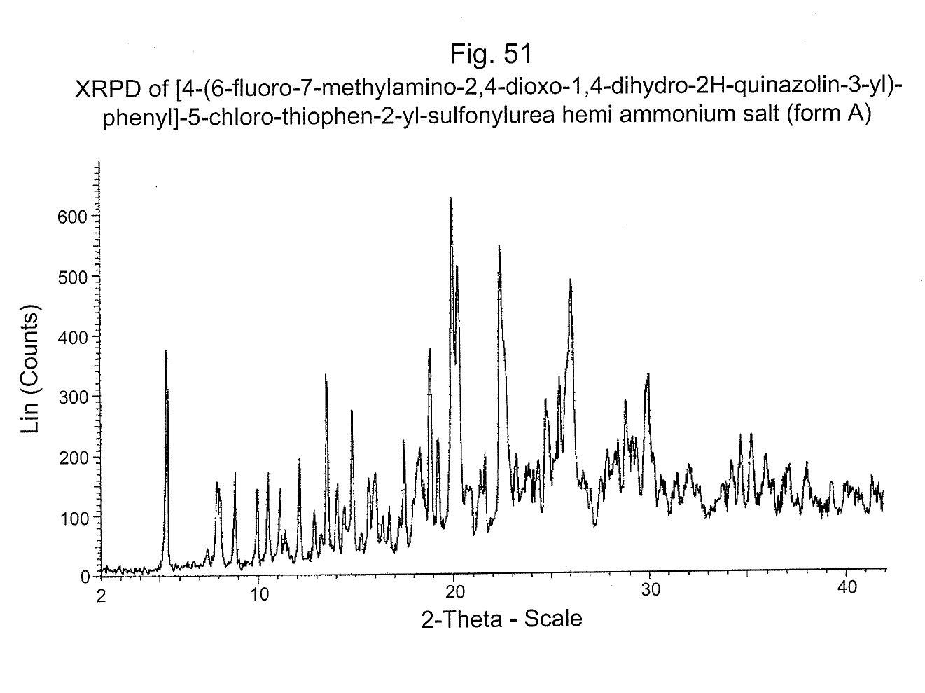

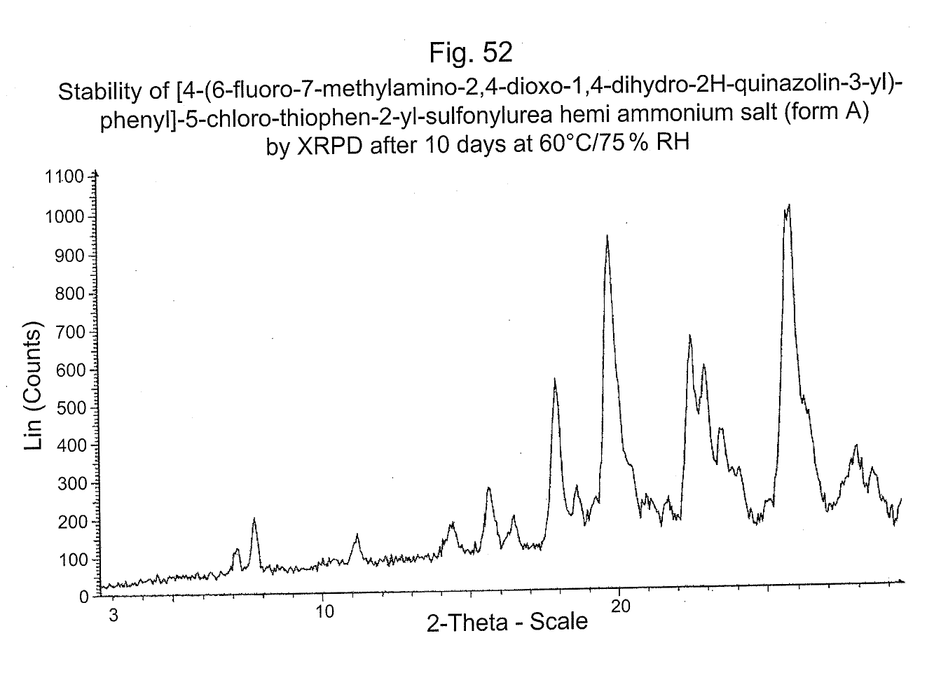

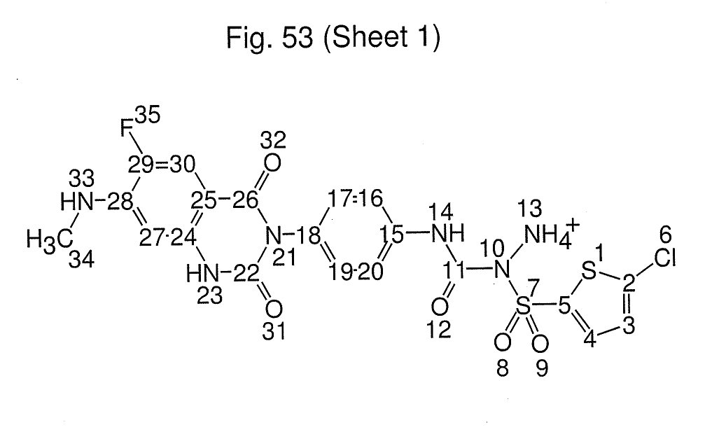

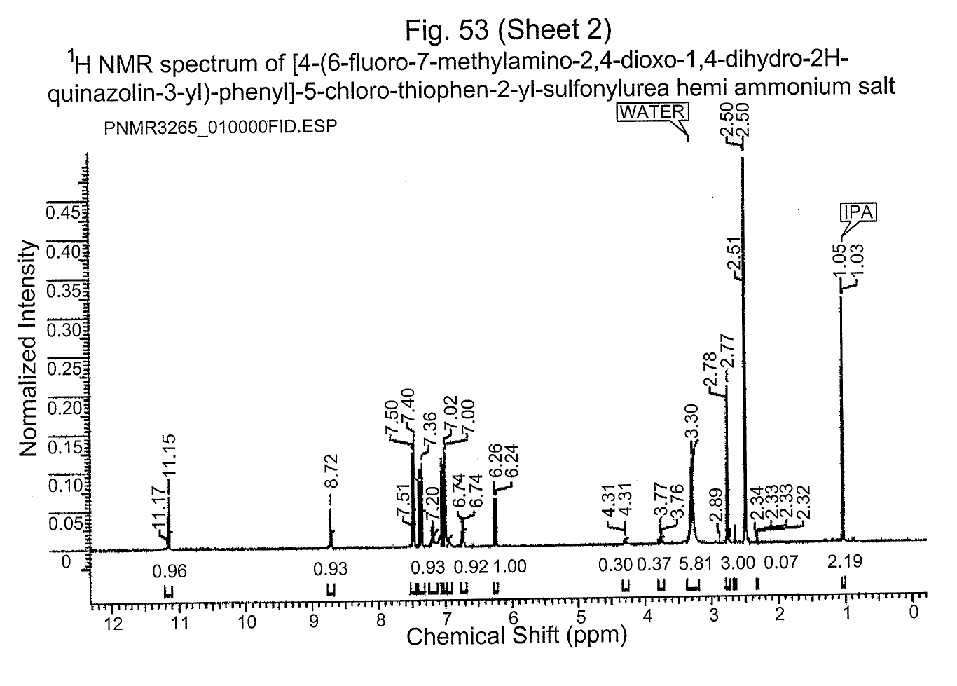

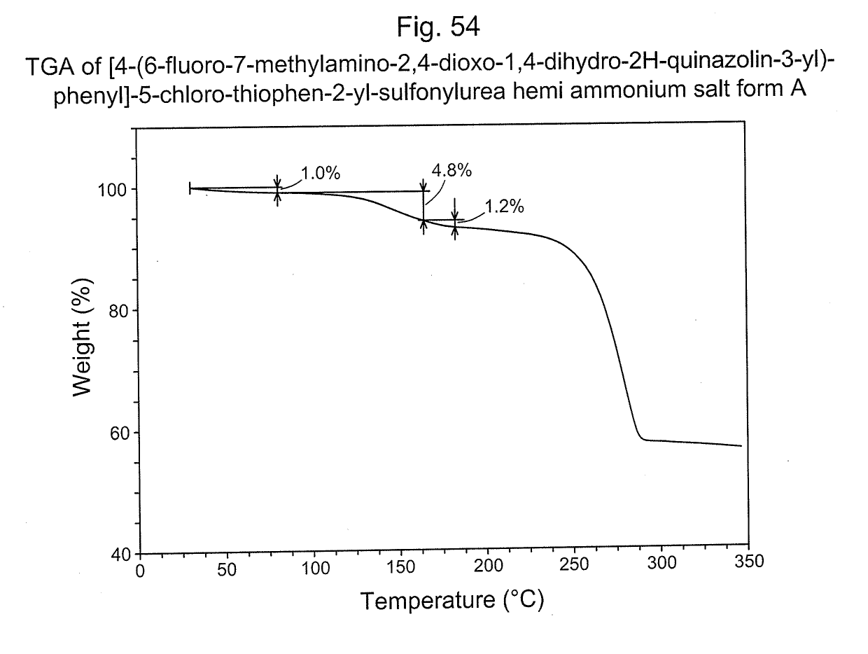

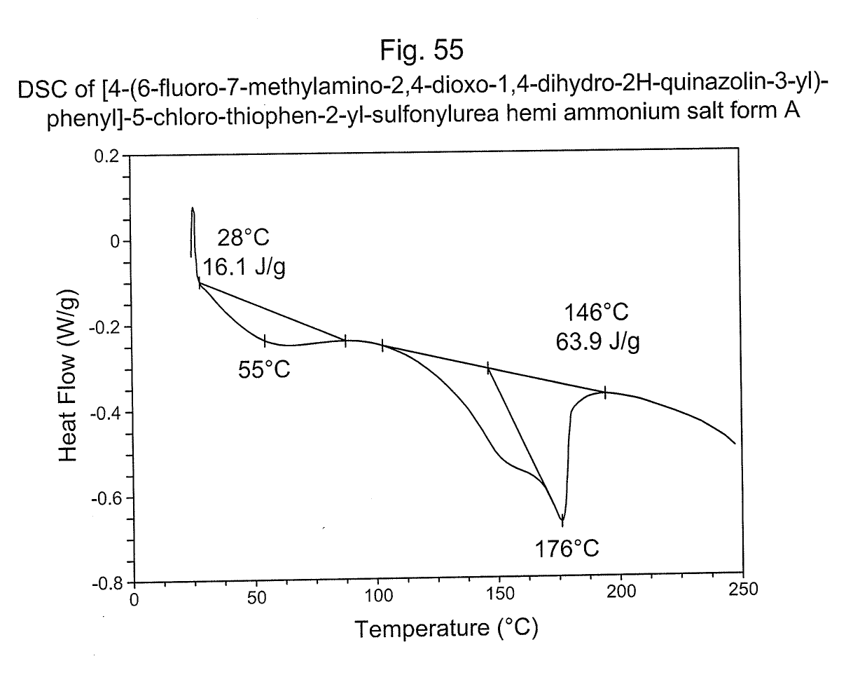

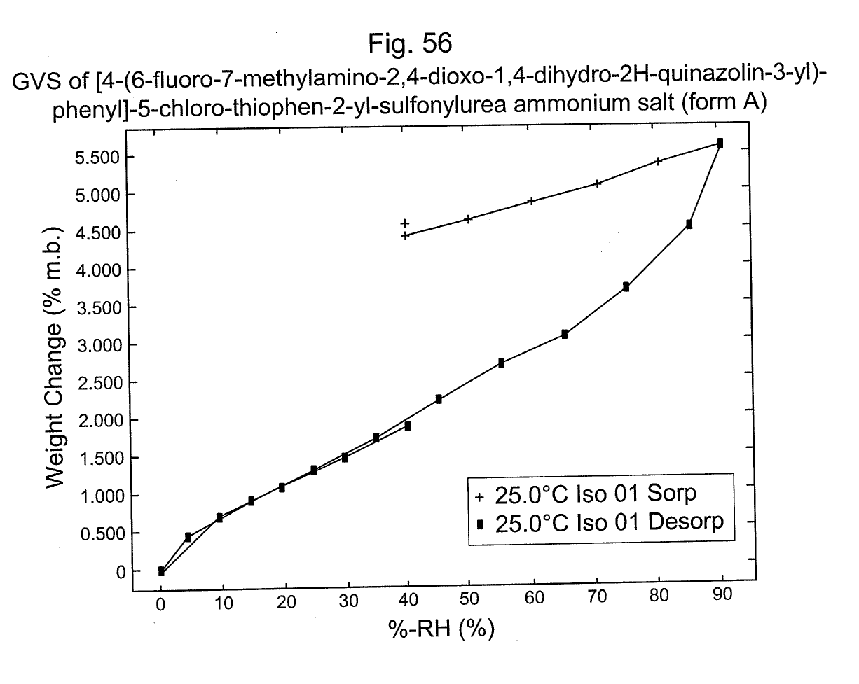

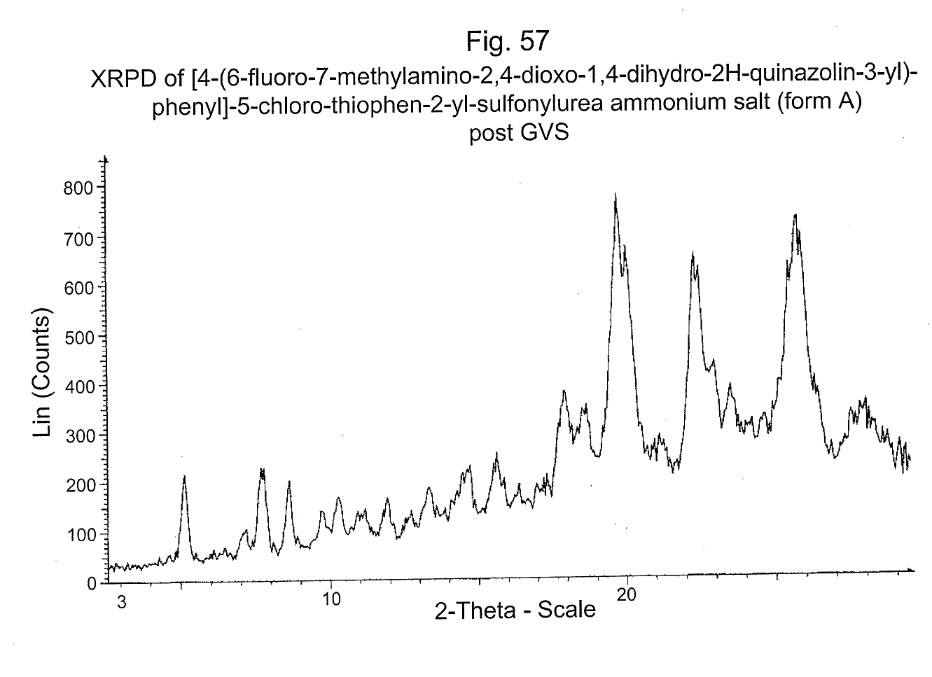

FIGS. 55 and 51 respectively show the DSC trace and the X-ray powder pattern for another crystalline solid form A. In the DSC trace, an endotherm onset at about 146° C. is noteworthy.

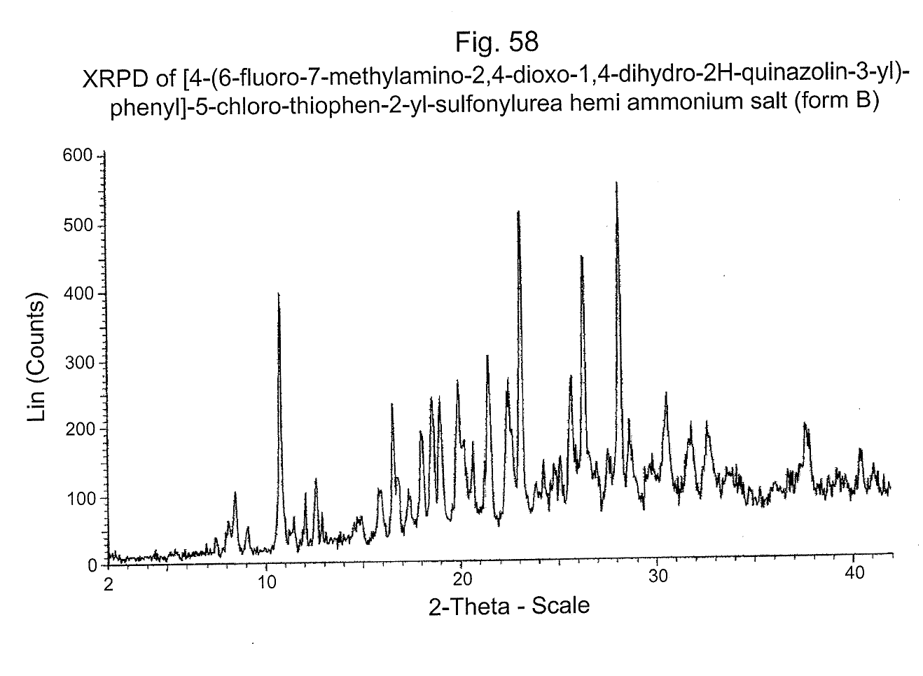

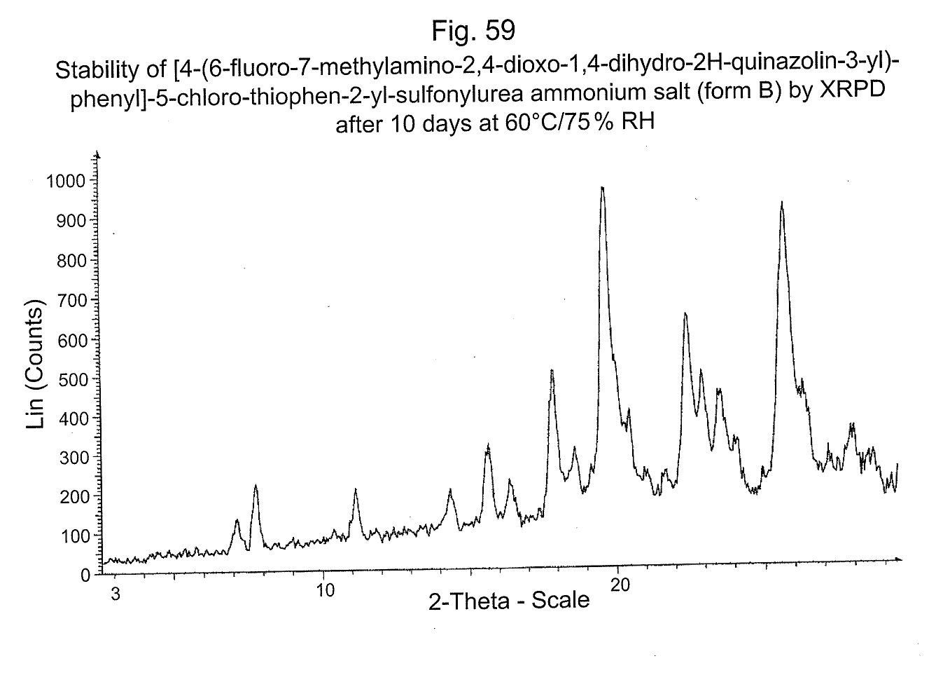

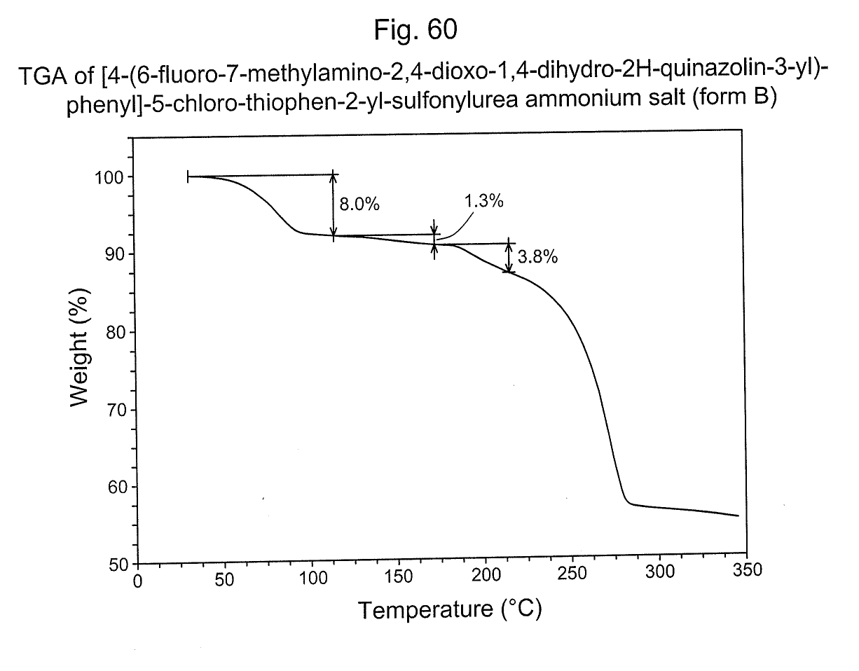

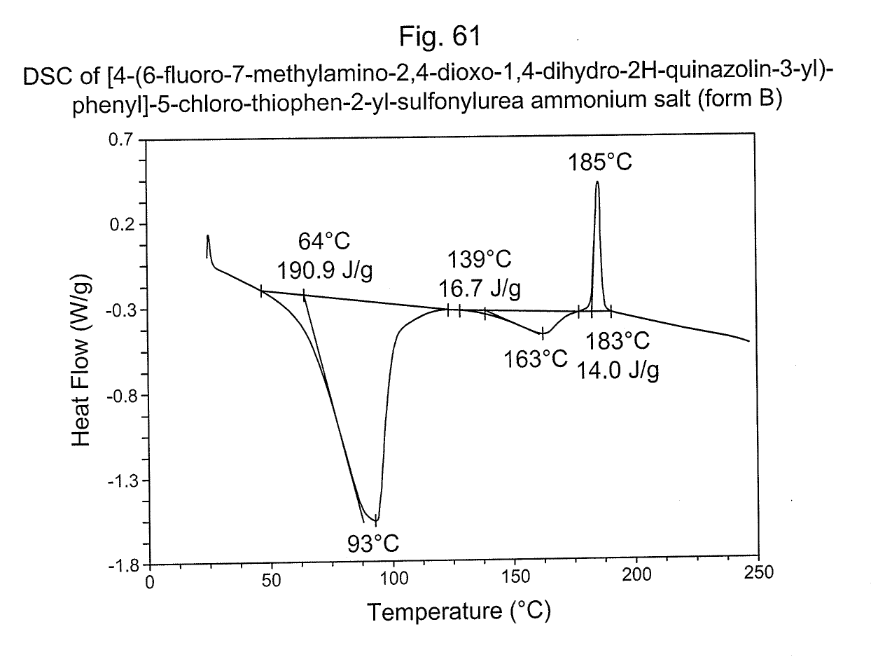

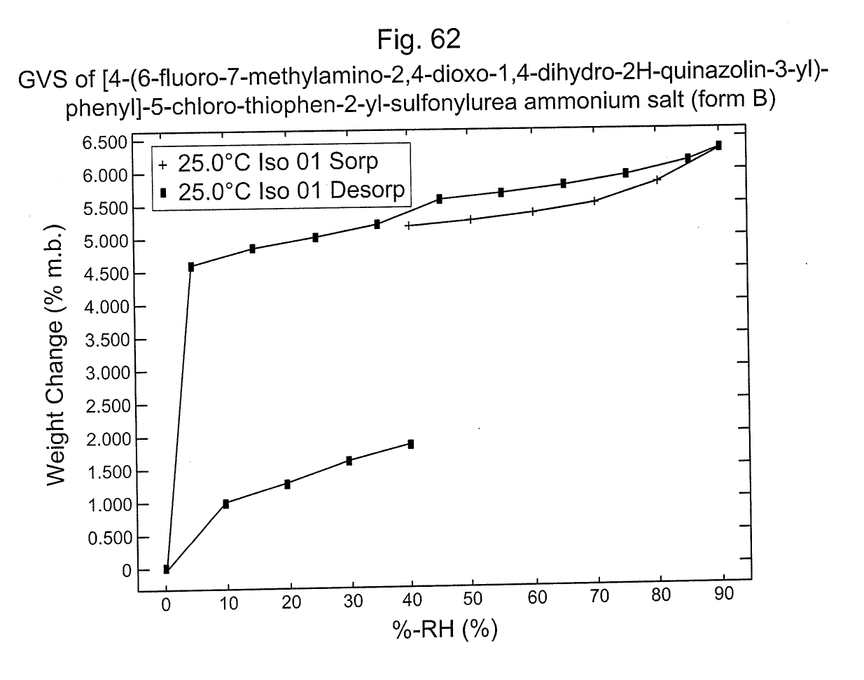

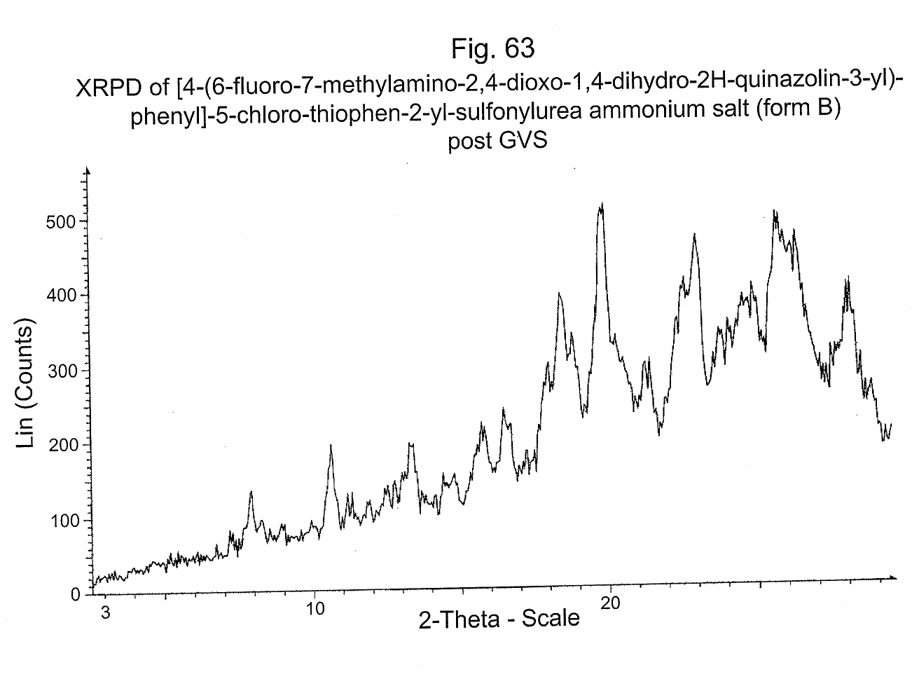

FIGS. 61 and 58 respectively show the DSC trace and the X-ray powder pattern for another crystalline solid form B. In the DSC trace, endotherms with onset temperatures at about 64° C. and 139° C. and an exotherm onset temperature at about 183° C. are noteworthy.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea ammonium salt in new crystalline forms designated as form A and form B.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea ammonium salt in a crystalline solid form, including a substantially pure form, which provides at least one of:

(i) an X-ray powder diffraction pattern substantially in accordance with FIG. 51; and
(ii) a DSC scan substantially in accordance with FIG. 55; herein designated as form A.

In another embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea ammonium salt in a crystalline solid form, including a substantially pure form, which provides a DSC maximum endotherm at about 146° C.; herein designated as form A.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea ammonium salt in a crystalline solid form, including a substantially pure form, which provides at least one of:

(i) an X-ray powder diffraction pattern substantially in accordance with FIG. 58; and
(ii) a DSC scan substantially in accordance with FIG. 61; herein designated as form B.

### L-lysine Salt Amorphous Form

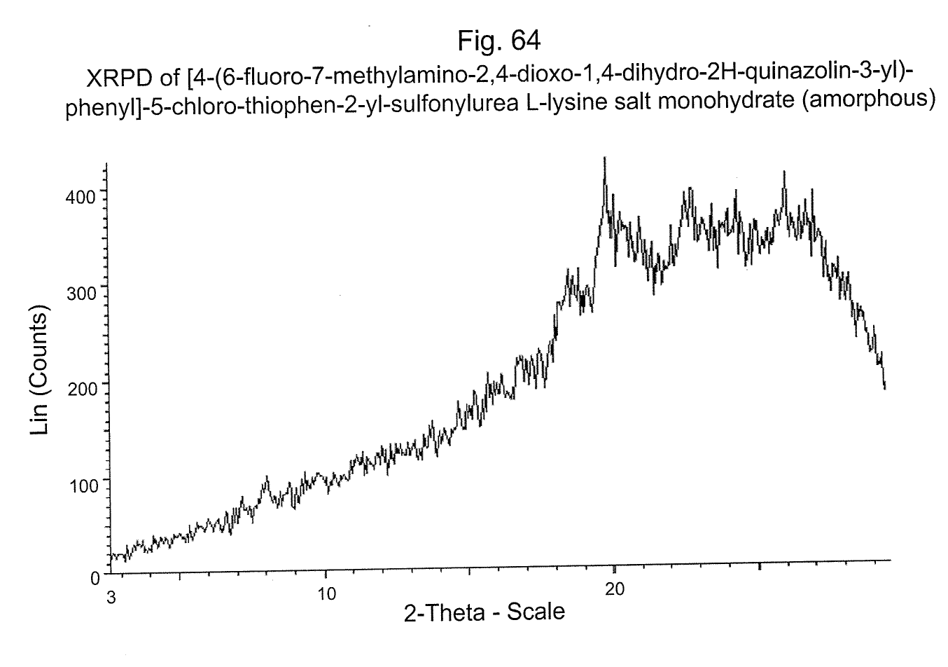

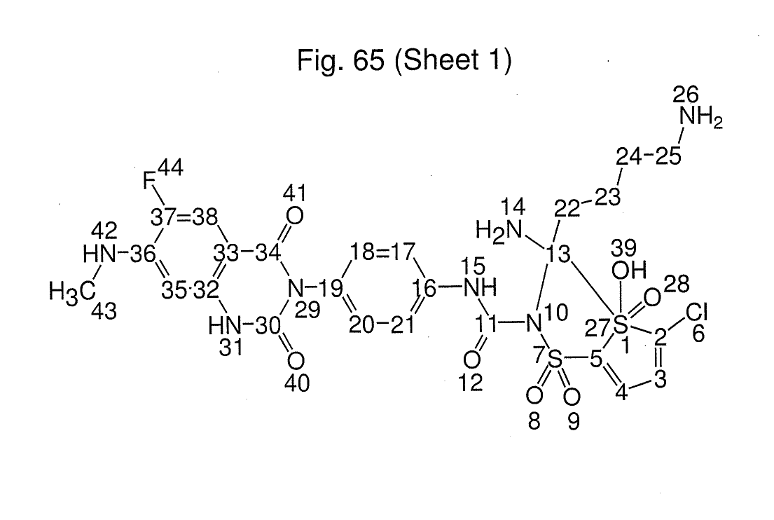

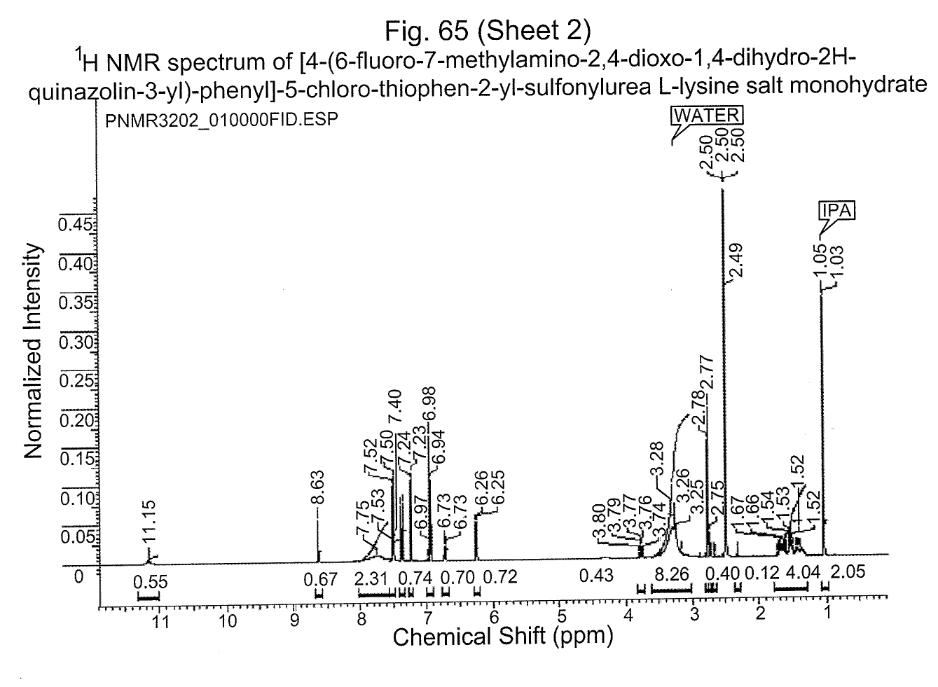

FIG. 64 shows the X-ray powder pattern for the amorphous form.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea L-lysine salt in an amorphous form.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea L-lysine salt in an amorphous form, including a substantially pure form, which provides an X-ray powder diffraction pattern substantially in accordance with FIG. 64; herein designated as amorphous.

### Magnesium Salt Form A

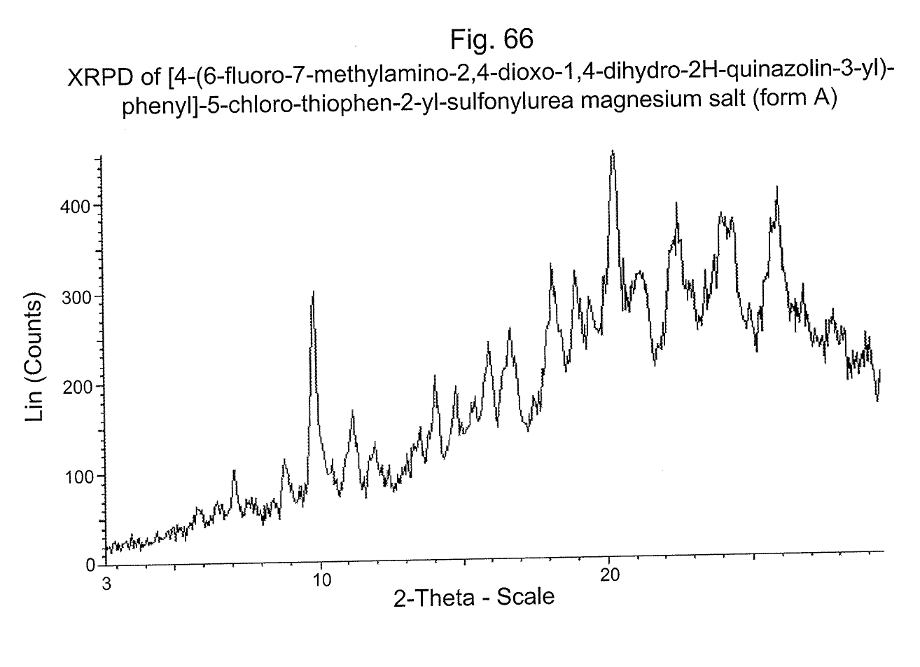

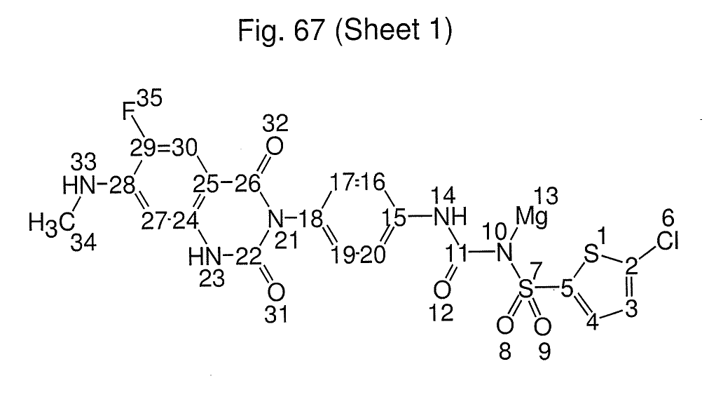

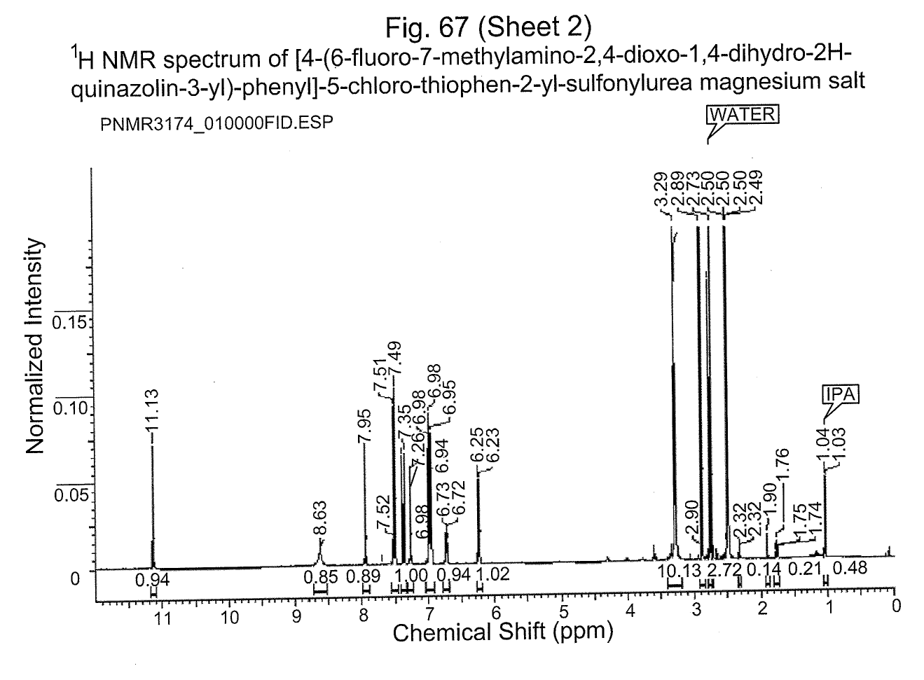

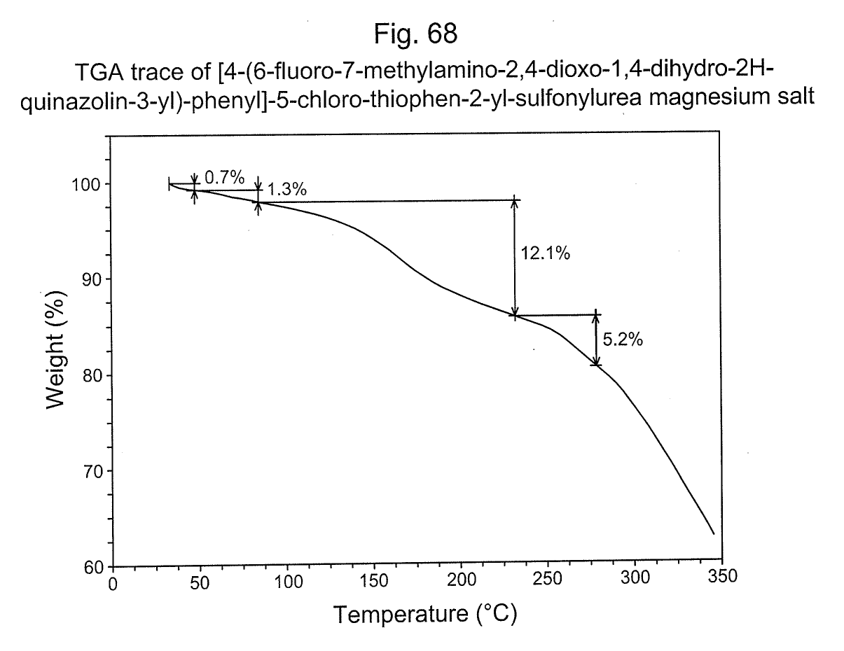

FIG. 66 shows the X-ray powder pattern for a crystalline solid form A.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea magnesium salt in a new crystalline form designated as form A.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea magnesium salt in a crystalline solid form, including a substantially pure form, which provides an X-ray powder diffraction pattern substantially in accordance with FIG. 66; herein designated as form A.

### L-Arginine Salt Amorphous Form

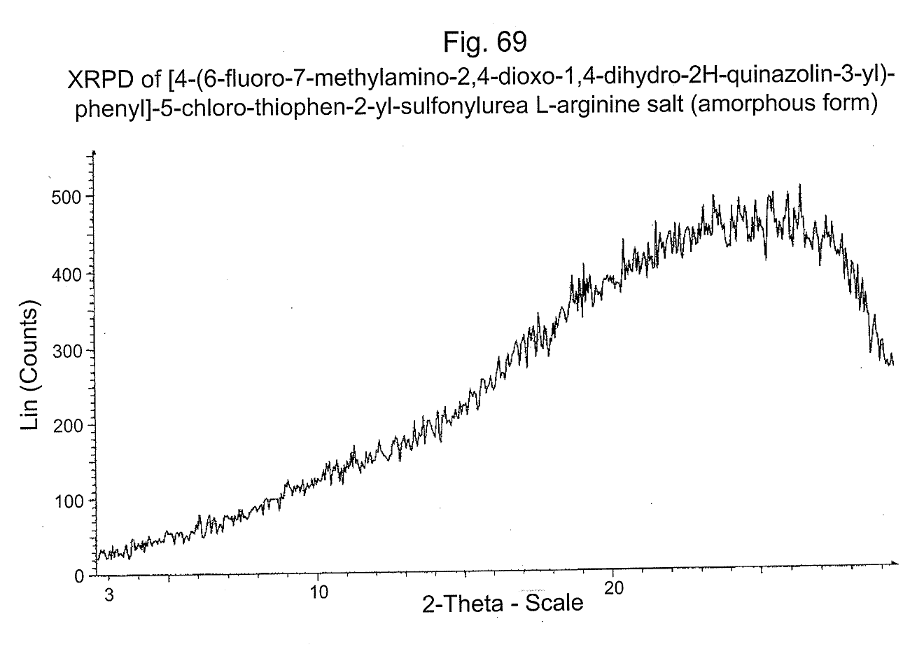

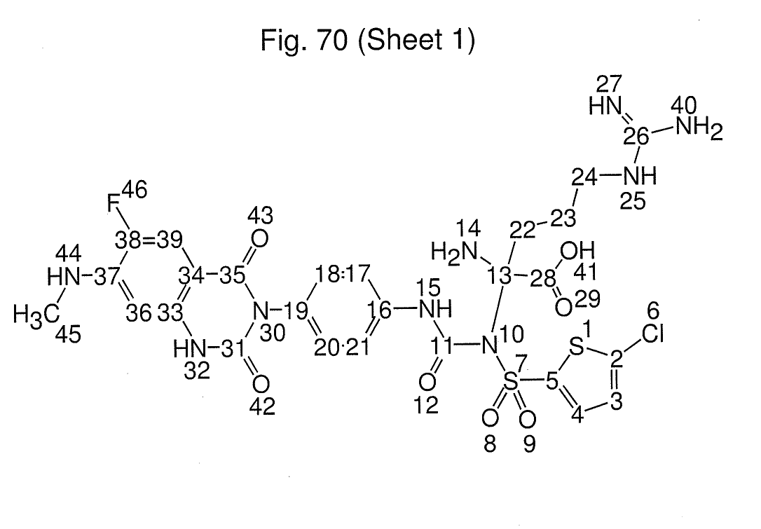

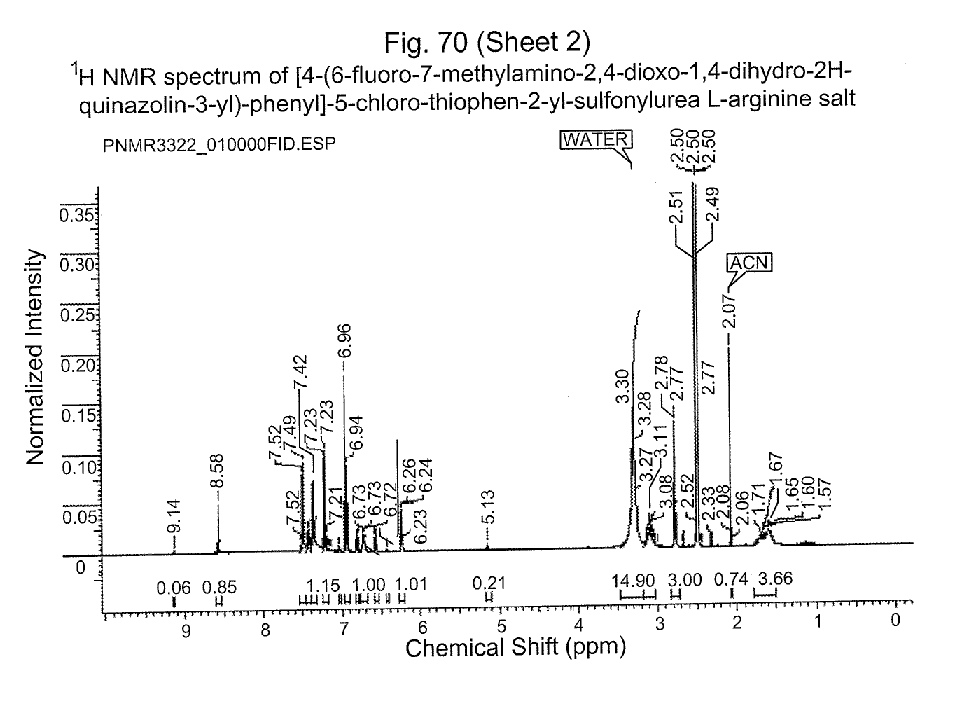

FIG. 69 shows the X-ray powder pattern for the amorphous form.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea L-arginine salt in an amorphous form.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea L-arginine in amorphous form, including a substantially pure form, which provides an X-ray powder diffraction pattern substantially in accordance with FIG. 69; herein designated as amorphous.

### N-ethylglucamine Salt Amorphous Form

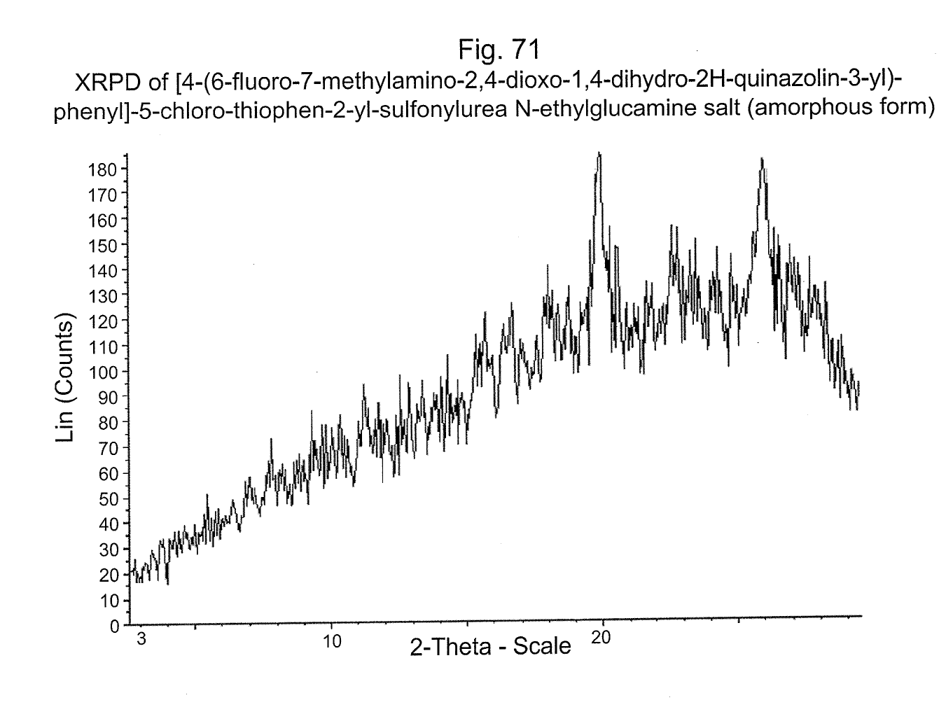

FIG. 71 shows the X-ray powder pattern for an amorphous form.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea N-ethylglucamine salt in an amorphous form.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea N-ethylglucamine in amorphous form, including a substantially pure form, which provides an X-ray powder diffraction pattern substantially in accordance with FIG. 71; herein designated as amorphous.

### N-methylglucamine Salt Amorphous Form

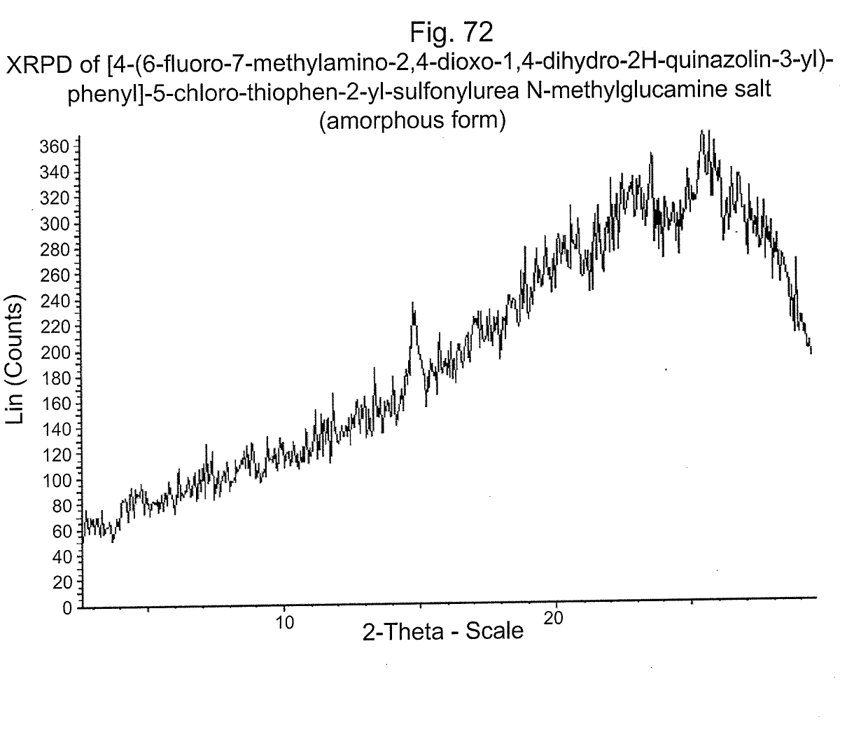

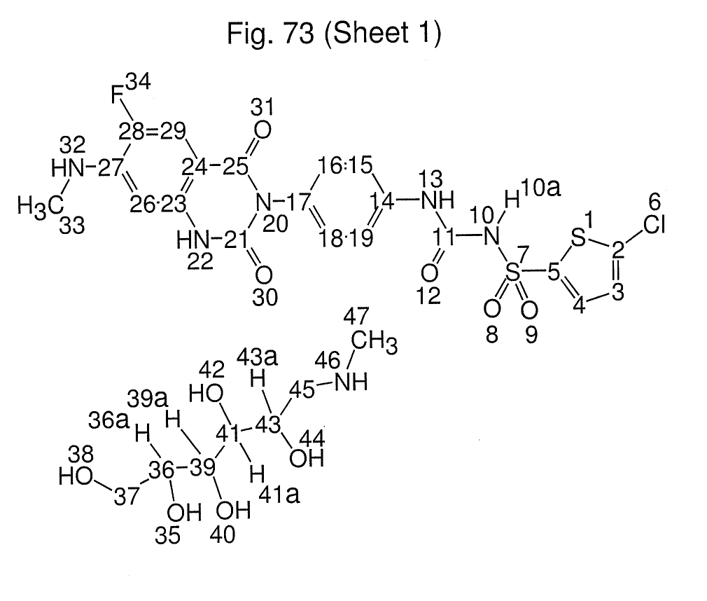

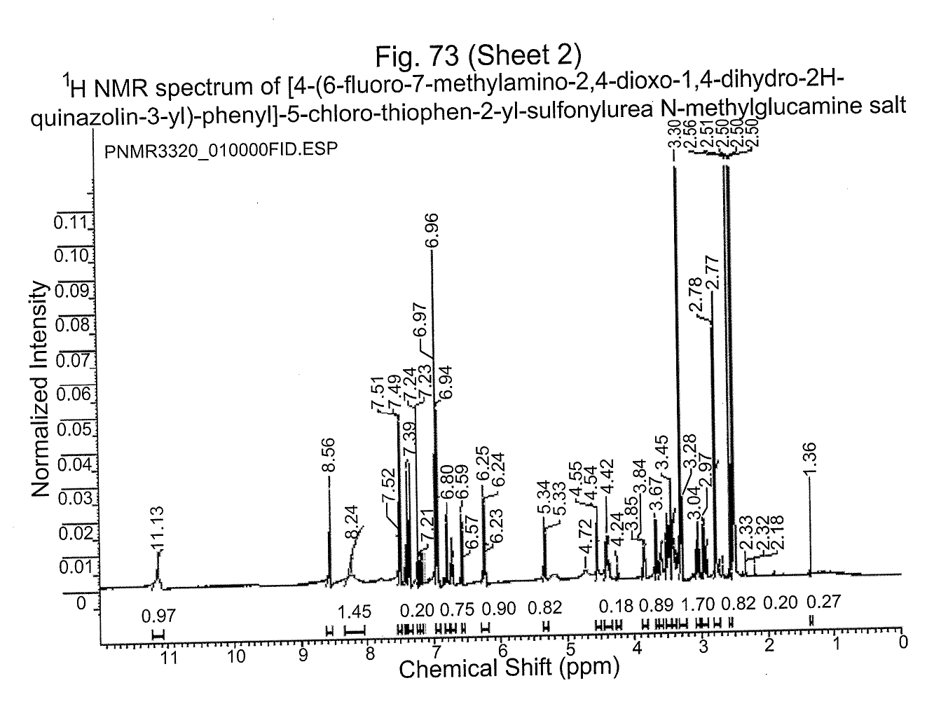

FIG. 72 shows the X-ray powder pattern for an amorphous form.

Thus in one embodiment, the present invention provides [4-(6-fluoro-7-methylamino-2,4-dioxo-1,4-dihydro-2H-quinazolin-3-yl)-phenyl]-5-chloro-thiophen-2-yl-sulfonylurea N-methylglucamine salt in an amorphous form.

Thus in one embodiment, the invention provides [4-(6-fluoro-7-methylamino-2,4-dioxo-1,4-dihydro-2H-quinazolin-3-yl)-phenyl]-5-chloro-thiophen-2-yl-sulfonylurea N-methylglucamine in an amorphous form, including a substantially pure form, which provides an X-ray powder diffraction pattern substantially in accordance with FIG. 72; herein designated as amorphous.

Crystalline form A of [4-(6-fluoro-7-methylamino-2,4-dioxo-1,4-dihydro-2H-quinazolin-3-yl)-phenyl]-5-chloro-thiophen-2-yl-sulfonylurea potassium salt is a 2.5 hydrate which is stable between 20-90% RH at 25° C. but which dehydrates between 20 and 0% RH at 25° C. Form A of the potassium salt has been found to be equally stable as the amorphous form of the sodium salt. No change in the chemical purity of either salt form was observed after one week when in accelerated stability tests at high temperature (40° C.) and high relative humidity (75% RH). An advantage of the potassium crystalline form A is that it is less hygroscopic than the amorphous form of the sodium salt which picks up >15% w/w water at 40% RH. Form B of the potassium salt is a hemi hydrate and non-hygroscopic. Form B of the potassium salt retains a better physical appearance and handling properties over a longer period of time. An improvement in the physical appearance of a dosage form of a drug enhances both physician and patient acceptance and increases the likelihood of success of the treatment.

Further embodiments of the invention include mixtures of the different crystalline solid forms, and the amorphous form, of [4-(6-fluoro-7-methylamino-2,4-dioxo-1,4-dihydro-2H-quinazolin-3-yl)-phenyl]-5-chloro-thiophen-2-yl-sulfonylurea and its salts. Such mixtures include compositions comprising at least one solid form or at least two solid forms selected from form A, form B, form C, form D and the amorphous form of the potassium salt. Any of the analytical techniques described herein may be used to detect the presence of the solid forms in such compositions. Detection may be done qualitatitvely, quantitatively, or semi-quantitatively as those terms as used and understood by those of skill in the solid-state analytical arts.

For these analyses, use of standard analytical techniques involving reference standards may be used. Further, such methods may include use of techniques such as least squares in conjunction with a spectroscopic analytical technique. These techniques may also be used in pharmaceutical compositions of the invention.

### V. Preparation of Crystalline Solid and Amorphous Forms of the Invention

Furthermore, the present invention is directed to processes for the preparation of crystalline solid and amorphous forms of [4-(6-fluoro-7-methylamino-2,4-dioxo-1,4-dihydro-2H-quinazolin-3-yl)-phenyl]-5-chloro-thiophen-2-yl-sulfonylurea potassium and sodium salts.

Crystalline solid and amorphous forms of the compounds of the invention may be prepared by various methods as outlined below. Other well-known crystallization procedures as well as modification of the procedures outline above may be utilized.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form A, which is obtained by at least one of:

(i) crystallizing [4-(6-fluoro-7-methylamino-2,4-dioxo-1,4-dihydro-2H-quinazolin-3-yl)-phenyl]-5-chloro-thiophen-2-yl-sulfonylurea potassium salt from at least one solvent selected from the group consisting of ethanol, methanol, and combinations thereof and drying such that the crystal contained some solvent;
(ii) recrystallisation by heating [4-(6-fluoro-7-methylamino-2,4-dioxo-1,4-dihydro-2H-quinazolin-3-yl)-phenyl]-5-chloro-thiophen-2-yl-sulfonylurea potassium salt in at least one solvent selected from the group consisting of ethanol, methanol, and combinations thereof; crystallizing at a temperature of from about 50° C. to −10° C. and drying until the crystals contained at least about 0.05% solvent.
(iii) heating [4-(6-fluoro-7-methylamino-2,4-dioxo-1,4-dihydro-2H-quinazolin-3-yl)-phenyl]-5-chloro-thiophen-2-yl-sulfonylurea with sodium hydroxide or sodium ethoxide in tetrahydrofuran; crystallizing at a temperature of from about 50° C. to 25° C. and drying until the crystals contained at least about 0.05% solvent.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form B, which is obtained by at least one of:

(i) heating [4-(6-fluoro-7-methylamino-2,4-dioxo-1,4-dihydro-2H-quinazolin-3-yl)-phenyl]-5-chloro-thiophen-2-yl-sulfonylurea potassium salt in a solvent combination of ethanol and water; crystallizing at a temperature of from about 50° C. to −10° C. and drying until the crystals contain less than 0.05% organic solvent;
(ii) crystallizing [4-(6-fluoro-7-methylamino-2,4-dioxo-1,4-dihydro-2H-quinazolin-3-yl)-phenyl]-5-chloro-thiophen-2-yl-sulfonylurea potassium salt from a solvent combination of ethanol and water and drying such that the crystal contained less than 0.05% organic solvent; and
(iii) heating [4-(6-fluoro-7-methylamino-2,4-dioxo-1,4-dihydro-2H-quinazolin-3-yl)-phenyl]-5-chloro-thiophen-2-yl-sulfonylurea in potassium hydroxide or potassium ethoxide in isopropanol or a solvent combination of acetonitrile and water; crystallizing at a temperature of from about 50° C. to 4° C. and drying until the crystals contain less than 0.05% organic solvent.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form C, which is obtained by at least one of:

(i) [4-(6-fluoro-7-methylamino-2,4-dioxo-1,4-dihydro-2H-quinazolin-3-yl)-phenyl]-5-chloro-thiophen-2-yl-sulfonylurea was treated with 1.15 equivalent of potassium ethoxide in water; and heated for 50° C. for 2 hours followed by cooling to 4° C. and drying.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea potassium salt in a crystalline solid form D, which is obtained by at least one of:

(i) [4-(6-fluoro-7-methylamino-2,4-dioxo-1,4-dihydro-2H-quinazolin-3-yl)-phenyl]-5-chloro-thiophen-2-yl-sulfonylurea was treated with 1.15 equivalent of potassium hydroxide or potassium ethoxide in THF; and heated for 50° C. for 2 hours followed by cooling to 4° C. and drying.

In another embodiment of the present invention there is provided an amorphous form of [4-(6-fluoro-7-methylamino-2,4-dioxo-1,4-dihydro-2H-quinazolin-3-yl)-phenyl]-5-chloro-thiophen-2-yl-sulfonylurea potassium salt by triturating in isopropanol and drying.

In another embodiment of the present invention there is provided an amorphous form of [4-(6-fluoro-7-methylamino-2,4-dioxo-1,4-dihydro-2H-quinazolin-3-yl)-phenyl]-5-chloro-thiophen-2-yl-sulfonylurea sodium salt which is obtained by at least one of:

(i) heating [4-(6-fluoro-7-methylamino-2,4-dioxo-1,4-dihydro-2H-quinazolin-3-yl)-phenyl]-5-chloro-thiophen-2-yl-sulfonylurea sodium salt in at least one solvent selected from the group consisting of isopropanol, acetonitrile, ethanol and combinations thereof; and precipitating at a temperature of from about 50° C. to −10° C.;
(ii) precipitating [4-(6-fluoro-7-methylamino-2,4-dioxo-1,4-dihydro-2H-quinazolin-3-yl)-phenyl]-5-chloro-thiophen-2-yl-sulfonylurea sodium salt from at least one solvent selected from the group consisting of isopropanol, acetonitrile, ethanol and combinations thereof.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form A, which is obtained by at least one of:

(iii) heating [4-(6-fluoro-7-methylamino-2,4-dioxo-1,4-dihydro-2H-quinazolin-3-yl)-phenyl]-5-chloro-thiophen-2-yl-sulfonylurea with sodium hydroxide in tetrahydrofuran or isopropanol at 50° C. followed by cooling to 25° C., filtering and drying to give sodium salt Form A.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form B, which is obtained by at least one of:

(iii) heating [4-(6-fluoro-7-methylamino-2,4-dioxo-1,4-dihydro-2H-quinazolin-3-yl)-phenyl]-5-chloro-thiophen-2-yl-sulfonylurea with sodium ethoxide in isopropanol at 50° C. followed by cooling to 25° C., filtering and drying to give sodium salt Form B.

In another embodiment of the present invention there is provided [4-(6-fluoro-7-methylamino-2,4-dioxo-1,4-dihydro-2H-quinazolin-3-yl)-phenyl]-5-chloro-thiophen-2-yl-sulfonylurea sodium salt in a crystalline solid form C, which is obtained by at least one of:

(iii) heating [4-(6-fluoro-7-methylamino-2,4-dioxo-1,4-dihydro-2H-quinazolin-3-yl)-phenyl]-5-chloro-thiophen-2-yl-sulfonylurea with sodium ethoxide in isopropanol at 50° C. followed by cooling to 25° C., filtering and drying to give sodium salt Form C.

Furthermore, the present invention is directed to the above described processes for the preparation of crystalline solid and amorphous forms of [4-(6-fluoro-7-methylamino-2,4-dioxo-1,4-dihydro-2H-quinazolin-3-yl)-phenyl]-5-chloro-thiophen-2-yl-sulfonylurea potassium and sodium salts.

[4-(6-fluoro-7-methylamino-2,4-dioxo-1,4-dihydro-2H-quinazolin-3-yl)-phenyl]-5-chloro-thiophen-2-yl-sulfonylurea in a crystalline solid or amorphous form may be prepared by various methods as further described below in the Examples. The examples illustrate, but do not limit the scope of the present invention. [4-(6-fluoro-7-methylamino-2,4-dioxo-1,4-dihydro-2H-quinazolin-3-yl)-phenyl]-5-chloro-thiophen-2-yl-sulfonylurea in crystalline solid or amorphous forms may be isolated using typical isolation and purification techniques known in the art, including, for example, chromatographic, and other procedures as well as modification of the procedures outlined above.

In formulating [4-(6-fluoro-7-methylamino-2,4-dioxo-1,4-dihydro-2H-quinazolin-3-yl)-phenyl]-5-chloro-thiophen-2-yl-sulfonylurea potassium salt to prepare immediate release beads, i.e., using wet granulation followed by extrusion, spherinization and drying, the bead dissolution was slow and incomplete. The XRPD pattern of the beads after compensating for the background signal was not consistent with form B of [4-(6-fluoro-7-methylamino-2,4-dioxo-1,4-dihydro-2H-quinazolin-3-yl)-phenyl]-5-chloro-thiophen-2-yl-sulfonylurea potassium salt, the starting API form or the known form of free acid.

A grinding experiment using a mortar and pestle to mimic wet granulation conditions was performed. Form B of [4-(6-fluoro-7-methylamino-2,4-dioxo-1,4-dihydro-2H-quinazolin-3-yl)-phenyl]-5-chloro-thiophen-2-yl-sulfonylurea potassium salt was ground with either 35% or 90% of water for approximately 10 min and then dried in oven at 40° C. overnight. The XRPD result of the sample ground with 90% of water gave a completely different XRPD pattern which was consistent with the form of the API in the beads. The sample ground with 35% of water gave a similar XRPD pattern to form B. The samples were also run by TGA and DSC. Grinding form B with 75% of water for 10-20 min. and analysis gave XRPD data which indicated that the API has converted to an amorphous form. The same sample was analyzed for XRPD again after 1 month storage at ambient room temperature. Based on XRPD results, the material had converted to form C. This result suggests that the conversion of form B to form C probably proceeds via an amorphous phase.

It was observed that after grinding the API with the diluent of Tox formulation, i.e., 0.5% methylcellulose and 0.1M phosphate sodium buffer at pH7.4, the drug particles became very dense and quickly precipitated during transfer and dosing. The procedure was repeated and it was also found that the suspended particles quickly coalesced and formed clumps which became difficult to redisperse. Work was carried out to identify a vehicle and preparation procedure that does not cause coalescence and solid state conversion. It was found that by removing 0.5% methylcellulose from the formulation, the irreversible coalescence problem can be solved. In addition, if only dry grinding is used to reduce the particle size of the API first and subsequently, the API is quickly dispersed into aqueous 0.1M phosphate buffer without applying significant mechanical stress, the solid form of the API can be maintained in form B for at least 6 hours.

A second lot of form C was prepared by repetitive grinding with more than 90% w/w water followed by drying in a 40° C. oven for at least 2 hours. During different stages of the preparation, the solid state of the API was followed by DSC and TGA.

Other methods of preparing amorphous and crystalline forms of salts of the invention are provided in the Examples.

### VI. Pharmaceutical Compositions

A salt of formula (I) according to the invention may be formulated into pharmaceutical compositions. Accordingly, the invention also provides a pharmaceutical composition for preventing or treating thrombosis in a mammal, particularly those pathological conditions involving platelet aggregation, containing a therapeutically effective amount of a salt of formula (I) or a pharmaceutically acceptable salt thereof, each as described above, and a pharmaceutically acceptable carrier or agent. Preferably, a pharmaceutical composition of the invention contains a salt of formula (I), or a form thereof, in an amount effective to inhibit platelet aggregation, more preferably, ADP-dependent aggregation, in a mammal, in particular, a human. Pharmaceutically acceptable carriers or agents include those known in the art and are described below.

Pharmaceutical compositions of the invention may be prepared by mixing the salt of formula (I) with a physiologically acceptable carrier or agent. Pharmaceutical compositions of the invention may further include excipients, stabilizers, diluents and the like and may be provided in sustained release or timed release formulations. Acceptable carriers, agents, excipients, stablilizers, diluents and the like for therapeutic use are well known in the pharmaceutical field, and are described, for example, in Remington's Pharmaceutical Sciences, Mack Publishing Co., ed. A. R. Gennaro (1985). Such materials are non-toxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, di-saccharides, and other carbohydrates including cellulose or its derivatives, glucose, mannose or dextrins, chelating agents such as EDTA, sugar alcohols such as mannitol or sorbitol, counterions such as sodium and/or non-ionic surfactants such as TWEEN, or polyethyleneglycol.

Further embodiments of the invention include pharmaceutical compositions of [4-(6-fluoro-7-methylamino-2,4-dioxo-1,4-dihydro-2H-quinazolin-3-ye-phenyl]-5-chloro-thiophen-2-yl-sulfonylurea, its salts and forms, including in therapeutically effective amounts crystalline and amorphous forms of the salts disclosed herein. Said amounts of the at least one of said forms may or may not be in therapeutically effective amounts. Such pharmaceutical compositions may be in the form of a solid oral composition such as a tablet or a capsule or as a dry powder for inhalation.

Wet granulation is an important method to prepare solid oral pharmaceutical dosage forms. Form C of the potassium salt is a unique form that is generated during a wet granulation process. The presence of form C has hindered dissolution of spheronized beads which contain it until the beads were physically crushed. This hindered dissolution may be due to a specific interaction between form C and excipients in this particular formulation. Improved or at least equivalent dissolution behavior may be realized with different excipient compositions.

### Pharmaceutically Acceptable Carriers

Diagnostic applications of the salts of this invention will typically utilize formulations such as solutions or suspensions.

In the management of thrombotic disorders the salts of this invention may be utilized in compositions such as tablets, capsules, lozenges or elixirs for oral administration, suppositories, sterile solutions or suspensions or injectable administration, and the like, or incorporated into shaped articles. Subjects in need of treatment (typically mammalian subjects) can be administered appropriate dosages of the compounds of this invention that will provide optimal efficacy. The dose and method of administration will vary from subject to subject and be dependent upon such factors as the type of mammal being treated, its sex, weight, diet, concurrent medication, overall clinical condition, the particular salts employed, the specific use for which these salts are employed, and other factors which those skilled in the medical arts will recognize.

Capsules useful in the present invention can be prepared using conventional and known encapsulation techniques, such as that described in Stroud et al., U.S. Pat. No. 5,735,105. The capsule is typically a hollow shell of generally cylindrical shape having a diameter and length sufficient so that the pharmaceutical solution compositions containing the appropriate dose of the active agent fits inside the capsule. The exterior of the capsules can include plasticizer, water, gelatin, modified starches, gums, carrageenans, and mixtures thereof. Those skilled in the art will appreciate what compositions are suitable.

In addition to the active agent, tablets useful in the present invention can comprise fillers, binders, compression agents, lubricants, disintegrants, colorants, water, talc and other elements recognized by one of skill in the art. The tablets can be homogeneous with a single layer at the core, or have multiple layers in order to realize preferred release profiles. In some instances, the tablets of the instant invention may be coated, such as with an enteric coating. One of skill in the art will appreciate that other excipients are useful in the tablets of the present invention.

Lozenges useful in the present invention include an appropriate amount of the active agent as well as any fillers, binders, disintegrants, solvents, solubilizing agents, sweeteners, coloring agents and any other ingredients that one of skill in the art would appreciate is necessary. Lozenges of the present invention are designed to dissolve and release the active agent on contact with the mouth of the patient. One of skill in the art will appreciate that other delivery methods are useful in the present invention.

Formulations of the salts of this invention are prepared for storage or administration by mixing the salt having a desired degree of purity with physiologically acceptable carriers, excipients, stabilizers etc., and may be provided in sustained release or timed release formulations. Acceptable carriers or diluents for therapeutic use are well known in the pharmaceutical field, and are described, for example, in Remington's Pharmaceutical Sciences, Mack Publishing Co., (A. R. Gennaro Ed. 1985). Such materials are non-toxic to the recipients at the dosages and concentrations employed, and include buffers such as phosphate, citrate, acetate and other organic acid salts, antioxidants such as ascorbic acid, low molecular weight (less than about ten residues) peptides such as polyarginine, proteins, such as serum albumin, gelatin, or immunoglobulins, hydrophilic polymers such as polyvinylpyrrolidinone, amino acids such as glycine, glutamic acid, aspartic acid, or arginine, monosaccharides, disaccharides, and other carbohydrates including cellulose or its derivatives, glucose, mannose or dextrins, chelating agents such as EDTA, sugar alcohols such as mannitol or sorbitol, counterions such as sodium, and/or non-ionic surfactants such as Tween, Pluronics or polyethyleneglycol.

Dosage formulations of the salts of this invention to be used for therapeutic administration must be sterile. Sterility is readily accomplished by filtration through sterile membranes such as 0.2 micron membranes, or by other conventional methods. Formulations typically will be stored in lyophilized form or as an aqueous solution. The pH of the preparations of this invention typically will be between 3 and 11, more preferably from 5 to 9 and most preferably from 7 to 8. It will be understood that use of certain of the foregoing excipients, carriers, or stabilizers will result in the formation of cyclic polypeptide salts. While the preferred route of administration is by injection, other methods of administration are also anticipated such as intravenously (bolus and/or infusion), subcutaneously, intramuscularly, colonically, rectally, nasally or intraperitoneally, employing a variety of dosage forms such as suppositories, implanted pellets or small cylinders, aerosols, oral dosage formulations (such as tablets, capsules and lozenges) and topical formulations such as ointments, drops and dermal patches. The sterile of this invention are desirably incorporated into shaped articles such as implants which may employ inert materials such as biodegradable polymers or synthetic silicones, for example, Silastic, silicone rubber or other polymers commercially available.

The salts of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

The salts of this invention may also be delivered by the use of antibodies, antibody fragments, growth factors, hormones, or other targeting moieties, to which the salt molecules are coupled. The salts of this invention may also be coupled with suitable polymers as targetable drug carriers. Such polymers can include polyvinylpyrrolidinone, pyran copolymer, polyhydroxy-propyl-methacrylamide-phenol, polyhydroxyethyl-aspartamide-phenol, or polyethyleneoxide-polylysine substituted with palmitoyl residues. Furthermore, salts of the invention may be coupled to a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

### Dosing

Typically, about 0.5 to 500 mg of a salt or mixture of salts of this invention is compounded with a physiologically acceptable vehicle, carrier, excipient, binder, preservative, stabilizer, dye, flavor etc., as called for by accepted pharmaceutical practice. The amount of active ingredient in these compositions is such that a suitable dosage in the range indicated is obtained.

It is contemplated that a typical dosage will range from about 0.001 mg/kg to about 1000 mg/kg, preferably from about 0.01 mg/kg to about 100 mg/kg, and more preferably from about 0.10 mg/kg to about 20 mg/kg. The compounds of this invention may be administered once or several times daily and other dosage regimens may also be useful.

### VII. Methods of Treatment/Administration

### A. Preventing and Treating Disease Conditions Characterized by Undesired Thrombosis

Methods for preventing or treating thrombosis in a mammal embraced by the invention administering a therapeutically effective amount of a salt of formula (I) alone or as part of a pharmaceutical composition of the invention as described above to a mammal, in particular, a human. Compounds of formula (I) and pharmaceutical compositions of the invention containing a salt of formula (I) of the invention are suitable for use alone or as part of a multi-component treatment regimen for the prevention or treatment of cardiovascular diseases, particularly those related to thrombosis. For example, a compound or pharmaceutical composition of the invention may be used as a drug or therapeutic agent for any thrombosis, particularly a platelet-dependent thrombotic indication, including, but not limited to, acute myocardial infarction, unstable angina, chronic stable angina, transient ischemic attacks, strokes, peripheral vascular disease, preeclampsia/eclampsia, deep venous thrombosis, embolism, disseminated intravascular coagulation and thrombotic cytopenic purpura, thrombotic and restenotic complications following invasive procedures, e.g., angioplasty, carotid endarterectomy, post CABG (coronary artery bypass graft) surgery, vascular graft surgery, stent placements and insertion of endovascular devices and protheses, and hypercoagulable states related to genetic predisposition or cancers. In other groups of embodiments, the indication is selected from the group consisting of percutaneous coronary intervention (PCI) including angioplasty and/or stent, acute myocardial infarction (AMI), unstable angina (USA), coronary artery disease (CAD), transient ischemic attacks (TIA), stroke, peripheral vascular disease (PVD), Surgeries-coronary bypass and carotid endarectomy.

Compounds and pharmaceutical compositions of the invention may also be used as part of a multi-component treatment regimen in combination with other therapeutic or diagnostic agents in the prevention or treatment of thrombosis in a mammal. In certain preferred embodiments, compounds or pharmaceutical compositions of the invention may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin or anti-inflammatories (non-steriodal anti-inflammatories, cyclooxygenase II inhibitors). Co-administration may also allow for application of reduced doses of both the anti-platelet and the thrombolytic agents and therefore minimize potential hemorrhagic side-effects. Compounds and pharmaceutical compositions of the invention may also act in a synergistic fashion to prevent re-occlusion following a successful thrombolytic therapy and/or reduce the time to reperfusion.

The compounds and pharmaceutical compositions of the invention may be utilized in vivo, ordinarily in mammals such as primates, (e.g., humans), sheep, horses, cattle, pigs, dogs, cats, rats and mice, or in vitro. The biological properties, as defined above, of a compound or a pharmaceutical composition of the invention can be readily characterized by methods that are well known in the art such as, for example, by in vivo studies to evaluate antithrombotic efficacy, and effects on hemostasis and hematological parameters.

Compounds and pharmaceutical compositions of the invention may be in the form of solutions or suspensions. In the management of thrombotic disorders the compounds or pharmaceutical compositions of the invention may also be in such forms as, for example, tablets, capsules or elixirs for oral administration, suppositories, sterile solutions or suspensions or injectable administration, and the like, or incorporated into shaped articles. Subjects (typically mammalian) in need of treatment using the compounds or pharmaceutical compositions of the invention may be administered dosages that will provide optimal efficacy. The dose and method of administration will vary from subject to subject and be dependent upon such factors as the type of mammal being treated, its sex, weight, diet, concurrent medication, overall clinical condition, the particular salt of formula (I) employed, the specific use for which the compound or pharmaceutical composition is employed, and other factors which those skilled in the medical arts will recognize.

### B. Therapeutically Effective Amount

Dosage formulations of compounds of formula (I), or pharmaceutical compositions contain a compound of the invention, to be used for therapeutic administration must be sterile. Sterility is readily accomplished by filtration through sterile membranes such as 0.2 micron membranes, or by other conventional methods. Formulations typically will be stored in a solid form, preferably in a lyophilized form. While the preferred route of administration is orally, the dosage formulations of compounds of formula (I) or pharmaceutical compositions of the invention may also be administered by injection, intravenously (bolus and/or infusion), subcutaneously, intramuscularly, colonically, rectally, nasally, transdermally or intraperitoneally. A variety of dosage forms may be employed as well including, but not limited to, suppositories, implanted pellets or small cylinders, aerosols, oral dosage formulations and topical formulations such as ointments, drops and dermal patches. The compounds of formula (I) and pharmaceutical compositions of the invention may also be incorporated into shapes and articles such as implants which may employ inert materials such biodegradable polymers or synthetic silicones as, for example, SILASTIC, silicone rubber or other polymers commercially available. The compounds and pharmaceutical compositions of the invention may also be administered in the form of liposome delivery systems, such as small unilamellar vesicles, large unilamellar vesicles and multilamellar vesicles. Liposomes can be formed from a variety of lipids, such as cholesterol, stearylamine or phosphatidylcholines.

Therapeutically effective dosages may be determined by either in vitro or in vivo methods. For each particular compound or pharmaceutical composition of the present invention, individual determinations may be made to determine the optimal dosage required. The range of therapeutically effective dosages will be influenced by the route of administration, the therapeutic objectives and the condition of the patient. For injection by hypodermic needle, it may be assumed the dosage is delivered into the body's fluids. For other routes of administration, the absorption efficiency must be individually determined for each compound by methods well known in pharmacology. Accordingly, it may be necessary for the therapist to titer the dosage and modify the route of administration as required to obtain the optimal therapeutic effect. The determination of effective dosage levels, that is, the dosage levels necessary to achieve the desired result, will be readily determined by one skilled in the art. Typically, applications of compound are commenced at lower dosage levels, with dosage levels being increased until the desired effect is achieved.

The determination of effective dosage levels, that is, the dosage levels necessary to achieve the desired result, i.e., platelet ADP receptor inhibition, will be readily determined by one skilled in the art. Typically, applications of a compound or pharmaceutical composition of the invention are commenced at lower dosage levels, with dosage levels being increased until the desired effect is achieved. The compounds and compositions of the invention may be administered orally in an effective amount within the dosage range of about 0.01 to 1000 mg/kg in a regimen of single or several divided daily doses. If a pharmaceutically acceptable carrier is used in a pharmaceutical composition of the invention, typically, about 5 to 500 mg of a salt of formula (I) is compounded with a pharmaceutically acceptable carrier as called for by accepted pharmaceutical practice including, but not limited to, a physiologically acceptable vehicle, carrier, excipient, binder, preservative, stabilizer, dye, flavor, etc. The amount of active ingredient in these compositions is such that a suitable dosage in the range indicated is obtained.

### C. Administration

Therapeutic compound liquid formulations generally are placed into a container having a sterile access port, for example, an intravenous solution bag or vial having a stopper pierceable by hypodermic injection needle.

Typical adjuvants which may be incorporated into tablets, capsules, lozenges and the like are binders such as acacia, corn starch or gelatin, and excipients such as microcrystalline cellulose, disintegrating agents like corn starch or alginic acid, lubricants such as magnesium stearate, sweetening agents such as sucrose or lactose, or flavoring agents. When a dosage form is a capsule, in addition to the above materials it may also contain liquid carriers such as water, saline, or a fatty oil. Other materials of various types may be used as coatings or as modifiers of the physical form of the dosage unit. Sterile compositions for injection can be formulated according to conventional pharmaceutical practice. For example, dissolution or suspension of the active compound in a vehicle such as an oil or a synthetic fatty vehicle like ethyl oleate, or into a liposome may be desired. Buffers, preservatives, antioxidants and the like can be incorporated according to accepted pharmaceutical practice.

### D. Combination Therapies

The compounds of the present invention may also be used in combination with other therapeutic or diagnostic agents. In certain preferred embodiments, the compounds of this invention may be co-administered along with other compounds typically prescribed for these conditions according to generally accepted medical practice such as anticoagulant agents, thrombolytic agents, or other antithrombotics, including platelet aggregation inhibitors, tissue plasminogen activators, urokinase, prourokinase, streptokinase, heparin, aspirin, or warfarin. The compounds of the present invention may act in a synergistic fashion to prevent re-occlusion following a successful thrombolytic therapy and/or reduce the time to reperfusion. These compounds may also allow for reduced doses of the thrombolytic agents to be used and therefore minimize potential hemorrhagic side-effects. The compounds of this invention can be utilized in vivo, ordinarily in mammals such as primates, (e.g. humans), sheep, horses, cattle, pigs, dogs, cats, rats and mice, or in vitro.

It should be understood that the foregoing discussion, embodiments and examples merely present a detailed description of certain preferred embodiments. It will be apparent to those of ordinary skill in the art that various modifications and equivalents can be made without departing from the spirit and scope of the invention. All the patents, journal articles and other documents discussed or cited above are herein incorporated by reference.

The following preparations and examples are given to enable those skilled in the art to more clearly understand and to practice the present invention. They should not be considered as limiting the scope of the invention, but merely as being illustrative and representative thereof.

### VIII. Examples

Unless stated otherwise, the abbreviations used throughout the specification have the following meanings:

| | |
|---|---|
| Å | Angstrom |
| A % | total percent area |
| aq. | aqueous |
| AcN, ACN | acetonitrile, methyl cyanide |
| n-BuOAc | n-butyl acetate |
| s-BuOAc | s-butyl acetate |
| ca. | approximately |
| cm | centimeter |
| ClPh | chlorophenol |
| d | doublet |
| DCE | dichloroethane |
| DCM | dichloromethane, methylene chloride |
| DIPE | di-isopropylether |
| DMA | dimethyl acetamide |
| DMF | dimethyl formamide |
| DS | drug substance |
| DSC | differential scanning calorimetry |
| EDTA | ethylenediaminetetraacetic acid |
| Et₂O | di-ethyl ether |
| EtOAc | ethyl acetate |
| EtOH | ethanol, ethyl alcohol |
| eq. | equivalent |
| f.a. | free acid |
| f.b. | free base |
| g | gram |
| H₂O | water-distilled or HPLC grade |
| HPLC | high performance liquid chromatography |
| hr | hour |
| Hz | Hertz |
| IR | infrared |
| IPA | iso-propy alcohol, propan-2-ol |
| iPrOAc | iso-propyl acetate |
| iPrOH | iso-propy alcohol, propan-2-ol |
| J | coupling constant |
| kg | kilogram |
| kV | kilovolts |
| L | liter |
| LOD | limit of detection |
| LNB | Laboratory Note Book |
| MeCN | methyl cyanide, acetonitrile |
| MEK | methyl ethyl ketone, butanone |
| M | molar |
| m | multiplet |
| mA | milliampere |
| Me | methyl |
| MeOH | methanol, methyl alcohol |
| MIBK | methyl isobutyl ketone, 2,2-dimethyl butan-3-one |
| mg | milligram |
| min. | minute |
| mL | milliliter |
| mm | millimeter |
| MTBE | tertiary butyl methyl ether |
| nBuOH | n-butanol, butan-1-ol |
| N | normal |
| nM | nanomolar |
| NMP | n-methyl pyrrolidone |
| NMR | nuclear magnetic resonance |
| nPrOH | n-propanol, propan-1-ol |
| PF | Project Folder |
| PTFE | polytetrafluoroethene, polytetrafluoroethylene |
| RM | reaction mixture |
| RT | room temperature |
| s | singlet |
| SM | starting material |
| tBME/TBME | t-butyl methyl ether |
| tBuOH | t-butanol (2-methyl-propan-2-ol) |
| TDS | total dissolved solids |
| TGA | thermal gravimetric analysis |
| THF | tetrahydrofuran |
| TMP | 2,2,4-trimethylpentane, iso-octane |
| μM | micromolar |

### General Methods

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1967-2004, Volumes 1-22; Rodd's Chemistry of Carbon Compounds, Elsevier Science Publishers, 1989, Volumes 1-5 and Supplementals; and Organic Reactions, Wiley & Sons: New York, 2005, Volumes 1-65. The following synthetic reaction schemes are merely illustrative of some methods by which the compounds of the present invention can be synthesized, and various modifications to these synthetic reaction schemes can be made and will be suggested to one skilled in the art having referred to the disclosure contained in this Application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein preferably are conducted under an inert atmosphere at atmospheric pressure at a reaction temperature range of from about −78° C. to about 150° C., more preferably from about 0° C. to about 125° C., and most preferably and conveniently at about room (or ambient) temperature, e.g., about 20° C. to about 75° C.

Referring to the examples that follow, compounds of the present invention were synthesized using the methods described herein, or other methods, which are well known in the art.

The compounds and/or intermediates were characterized by high performance liquid chromatography (HPLC) using a Waters Alliance chromatography system with a 2695 Separation Module (Milford, Mass.). The analytical columns were C-18 SpeedROD RP-18E Columns from Merck KGaA (Darmstadt, Germany). Alternately, characterization was performed using a Waters Unity (HPLC) system with Waters Acquity UPLC BEH C-18 2.1 mm×15 mm columns. A gradient elution was used, typically starting with 5% acetonitrile/95% water and progressing to 95% acetonitrile over a period of 5 minutes for the Alliance system and 1 minute for the Acquity system. All solvents contained 0.1% trifluoroacetic acid (TFA). Compounds were detected by ultraviolet light (UV) absorption at either 220 or 254 nm. HPLC solvents were from EMD Chemicals, Inc. (Gibbstown, N.J.). In some instances, purity was assessed by thin layer chromatography (TLC) using glass backed silica gel plates, such as, for example, EMD Silica Gel 60 2.5 cm×7.5 cm plates. TLC results were readily detected visually under ultraviolet light, or by employing well known iodine vapor and other various staining techniques.

Mass spectrometric analysis was performed on one of two Agilent 1100 series LCMS instruments with acetonitrile/water as the mobile phase. One system using TFA as the modifier and measures in positive ion mode [reported as MH+, (M+1) or (M+H)+] and the other uses either formic acid or ammonium acetate and measures in both positive [reported as MH⁺, (M+1) or (M+H)⁺] and negative [reported as M−, (M−1) or (M−H)⁻] ion modes.

Nuclear magnetic resonance (NMR) analysis was performed on some of the compounds with a Varian 400 MHz NMR (Palo Alto, Calif.). The spectral reference was either TMS or the known chemical shift of the solvent.

The purity of some of the invention compounds is assessed by elemental analysis (Robertson Microlit, Madison N.J.).

Melting points are determined on a Laboratory Devices MeI-Temp apparatus (Holliston, Mass.).

Preparative separations were carried out using either an Sq16x or an Sg100c chromatography system and prepackaged silica gel columns all purchased from Teledyne Isco, (Lincoln, Nebr.). Alternately, compounds and intermediates were purified by flash column chromatography using silica gel (230-400 mesh) packing material, or by HPLC using a C-18 reversed phase column. Typical solvents employed for the Isco systems and flash column chromatography were dichloromethane, methanol, ethyl acetate, hexane, acetone, aqueous hydroxyamine and triethyl amine. Typical solvents employed for the reverse phase HPLC were varying concentrations of acetonitrile and water with 0.1% trifluoroacetic acid.

### Instrumental and Methodology Details for Solid Forms

### 1. FT Infrared Spectroscopy (FTIR)

Samples were studied on a Perkin-Elmer Spectrum One fitted with a Universal ATR sampling accessory and running Spectrum V5.0.1 software. The resolution was set to 4 cm⁻¹ and 16 scans were collected over the range 4000 cm⁻¹ to 400 cm⁻¹. Control and Analysis software: Spectrum v 5.0.1.

2. Differential Scanning calorimetry (DSC)

DSC data (thermograms) were collected on a TA instruments Q1000 equipped with a 50 position auto-sampler or a Mettler instrument model DSC 823e, equipped with a 34 position auto-sampler. The energy and temperature calibration standard for both instruments was certified indium. The method used for either instrument was that the samples were heated at a rate of 10° C./min from 10° C. to 250° C. A nitrogen purge was maintained over the sample at about 30 to 50 ml/min for the TA instrument and 50 ml/min for the Mettler instrument.

Between 0.5 and 3 mg of sample was used, unless otherwise stated, and all samples were sealed in an aluminum pan with a pinhole in the lid. The control software for the TA instrument was: Advantage for Q series v 2.2.0.248, Thermal Advantage Release 4.2.1. and the analysis software for the TA instrument was: Universal Analysis 2000 v 4.1D Build 4.1.0.16. The control and the analysis software for the Mettler DSC was: STARE v. 9.01.

### 3. Thermogravimetric Analysis (TGA)

TGA data (thermograms) were collected on a TA Instrument Q500 TGA with a 16 position auto-sampler, or a Mettler instrument model: TGA/SDTA 851e, with a 34 position auto-sampler. The TA instrument was temperature calibrated using certified Alumel, and the Mettler instrument with certified indium. The method used for both instruments was that the samples were heated at a rate of 10° C./minute from ambient temperature to 350° C. A nitrogen purge of about 50 to 60 ml/min was maintained over the sample.

When using the TA instrument, typically 5-30 mg of each sample was loaded onto a pre-tared platinum crucible and open aluminum DSC pan. The control software was: Advantage for Q series v 2.2.0.248, Thermal Advantage Release 4.2.1. and the analysis software was: Universal Analysis 2000 v 4.1D Build 4.1.0.16. When using the Mettler instrument typically 5-10 mg sample was placed in an open aluminum pan. The software for this instrument (instrument control and data analysis) was: STARE v. 9.01.

### 4. XRPD (X-Ray Powder Diffraction)

### Bruker AXS C2 GADDS Diffractometer

X-Ray Powder Diffraction patterns were collected on a Bruker AXS C2 GADDS diffractometer using Cu Kα radiation (40 kV, 40 mA), automated XYZ stage, laser video microscope for auto-sample positioning and a HiStar 2-dimensional area detector. X-ray optics consists of a single Göbel multilayer mirror coupled with a pinhole collimator of 0.3 mm.

The beam divergence, i.e. the effective size of the X-ray beam on the sample, was approximately 4 mm. A θ-θ continuous scan mode was employed with a sample—detector distance of 20 cm which gives an effective 20 range of 3.2°-29.7°. Typically the sample would be exposed to the X-ray beam for 120 seconds.

### Ambient Conditions

Samples run under ambient conditions were prepared as flat plate specimens using powder as received without grinding. Approximately 1-2 mg of the sample was lightly pressed on a glass slide or silicon wafer to obtain a flat surface.

### Single Crystal XRD (SCXRD)

Data were collected on a Bruker AXS 1K SMART CCD diffractometer or a Bruker-Nonius Kappa CCD equipped with an Oxford Cryosystems Cryostream cooling device. Structures were solved using either the SHELXS or SHELXD programs and refined with the SHELXL program as part of the Bruker AXS SHELXTL suite. Unless otherwise stated, hydrogen atoms attached to carbon were placed geometrically and allowed to refine with a riding isotropic displacement parameter. Hydrogen atoms attached to a heteroatom were located in a difference Fourier synthesis and were allowed to refine freely with an isotropic displacement parameter.

### 5. Gravimetric Vapor Sorption (GVS) Studies

Sorption isotherms were obtained using a Hiden IGASorp moisture sorption analyser, controlled by CFRSorp software. The sample temperature was maintained at 25° C. by a Huber re-circulating water bath. The humidity was controlled by mixing streams of dry and wet nitrogen, with a total flow rate of 250 ml·min⁻¹. The relative humidity was measured by a calibrated Vaisala RH probe (dynamic range of 0-95% RH), located near the sample. The weight change, (mass relaxation) of the sample as a function of % RH was constantly monitored by the microbalance (accuracy ±0.001 mg).

Typically 10-20 mg of sample was placed in a tared mesh stainless steel basket under ambient conditions. The sample was loaded and unloaded at 40% RH and 25° C. (typical room conditions).

A moisture sorption isotherm was performed as outlined below (2 scans giving 1 complete cycle). The standard isotherm was performed at 25° C. at 10% RH intervals over a 0-90% RH range.

| Parameters | Values |
|---|---|
| Adsorption-Scan 1 | 40-90 |
| Desorption/Adsorption-Scan 2 | 85-Dry, Dry-40 |
| Intervals (% RH) | 10 |
| Number of Scans | 2 |
| Flow rate (ml · min⁻¹) | 250 |
| Temperature (° C.) | 25 |
| Stability (° C. · min⁻¹) | 0.05 |
| Minimum Sorption Time (hours) | 1 |
| Maximum Sorption Time (hours) | 4 |
| Mode | AF2 |
| Accuracy (%) | 98 |

The software uses a least squares minimisation procedure together with a model of the mass relaxation, to predict an asymptotic value. The measured mass relaxation value must be within 5% of that predicted by the software, before the next % RH value is selected. The minimum equilibration time was set to 1 hour and the maximum to 4 hours.

The sample was recovered after completion of the isotherm and re-analysed by XRPD.

### 6. ¹H NMR

Spectra were collected on a Bruker 400 MHz equipped with auto sampler. Samples were prepared in d₆-DMSO, unless otherwise stated.

### 7. Purity Analysis

Purity analysis was performed on an Agilent HP1100 system equipped with a diode array detector and using ChemStation software v9.

| | Normal Phase | Reverse Phase | X |
|---|---|---|---|
| Type of method | Isocratic | Gradient | X |
| Column: | Betabasic C18, 5 μm, 150 × 4.6 mm | | |
| Column Temperature (° C.): | 25 | | |
| Injection (μl): | 5 | | |
| Detection: | 325 | | |
| Wavelength, Bandwidth(nm): | 90 | | |
| Flow Rate (ml · min−1): | 0.8 | | |
| Phase A: | formic acid v/v in water | | |
| Phase B: | Acetonitrile: water 90:10 with 0.1% v/v | | |
| | formic acid | | |
| Timetable: | Time (min) | % Phase A | % Phase B |
| | 0 | 90 | 10 |
| | 2 | 90 | 10 |
| | 17 | 10 | 90 |
| | 21 | 10 | 90 |
| | 21.3 | 90 | 10 |
| | 25 | 90 | 10 |
| | Normal Phase | Reverse Phase | X |
| Type of method | Isocratic | Gradient | X |
| Column: | Phenomenex Luna C18 (2), 150 × | | |
| | 4.6 mm, 5 μm | | |
| Column Temperature (° C.): | 25 | | |
| Injection (μl): | 5 | | |
| Detection: | 255 | | |
| Wavelength, Bandwidth(nm): | 90 | | |
| Flow Rate (ml · min − 1): | 0.8-1.0 | | |
| Phase A: | 0.1% TFA v/v in water | | |
| Phase B: | 0.085% TFA in acetonitrile | | |
| Timetable: | Time (min) | % Phase A | % Phase B |
| | 0 | 95 | 5 |
| | 25 | 5 | 95 |
| | 25.2 | 95 | 5 |
| | 30 | 95 | 5 |

### 8 Polarised Light Microscopy (PLM)

Samples were studied on a Leica LM/DM polarised light microscope with a digital video camera for image capture. A small amount of each sample was placed on a glass slide, mounted in immersion oil and covered with a glass slip, the individual particles being separated as well as possible. The sample was viewed with appropriate magnification and partially polarised light, coupled to a λ false-colour filter.

### 9 Hot Stage Microscopy (HSM)

Hot Stage Microscopy was carried out using a Leica LM/DM polarised light microscope combined with a Mettler-Toledo MTFP82HT hot-stage and a digital video camera for image capture A small amount of each sample was placed onto a glass slide with individual particles separated as well as possible. The sample was viewed with appropriate magnification and partially polarised light, coupled to a λ false-colour filter, whilst being heated from ambient temperature typically at 10-20° C.·min⁻¹.

### 10 Water Determination by Karl Fischer

The water content of each sample was measured on a Mettler Toledo DL39 Coulometer using Hydranal Coulomat AG reagent and an argon purge. Weighed solid samples were introduced into the vessel on a platinum TGA pan which was connected to a subaseal to avoid water ingress. Approx 10 mg of sample was used per titration and duplicate determination were made.

### 11 Aqueous Solubility

Aqueous solubility was determined by suspending sufficient compound in 0.25 ml of water to give a maximum final concentration of ≧10 mg·ml⁻¹ of the parent free-form of the compound. The suspension was equilibrated at 25° C. for 24 hours then the pH was measured. The suspension was then filtered through a glass fibre C filter into a 96 well plate. The filtrate was then diluted by a factor of 101. Quantitation was by HPLC with reference to a standard solution of approximately 0.1 mg·ml⁻¹. in DMSO. Different volumes of the standard, diluted and undiluted sample solutions were injected. The solubility was calculated using the peak areas determined by integration of the peak found at the same retention time as the principal peak in the standard injection.

If there was sufficient solid in the filter plate, the XRPD pattern was collected.

| Type of method: | Reverse phase with gradient elution | | |
|---|---|---|---|
| Column: | Phenomenex Luna, C18 (2) 5 μm 50 × | | |
| | 4.6 mm | | |
| Column Temperature (° C.): | 25 | | |
| Injection (μl): | 5, 8 and 50 | | |
| Detection: | 260, 80 | | |
| Wavelength, Bandwidth (nm): | | | |
| Flow Rate (ml · min⁻¹): | 2 | | |
| Phase A: | 0.1% TFA in water | | |
| Phase B: | 0.085% TFA in acetonitrile | | |
| Timetable: | Time (min) | % Phase A | % Phase B |
| | 0.0 | 95 | 5 |
| | 1.0 | 80 | 20 |
| | 2.3 | 5 | 95 |
| | 3.3 | 5 | 95 |
| | 3.5 | 95 | 5 |
| | 4.4 | 95 | 5 |

### 12 Ion Chromatography

Data were collected on a Metrohm 861 Advanced Compact IC using IC Net software v2.3. Samples were prepared as 1000 ppm stocks in water. Where sample solubility was low, a suitable solvent such as DMSO was used. Samples were diluted to 50 ppm or 100 ppm with an appropriate solvent prior to testing. Quantification was achieved by comparison with standard solutions of known concentration of the ion being analysed.

| Type of method | Anion exchange |
|---|---|
| Column: | Metrosep A Supp 5-250 (4.0 × 250 mm) |
| Column Temperature (° C.): | Ambient |
| Injection (μl): | 20 |
| Detection: | Conductivity detector |
| Flow Rate (ml · min⁻¹): | 0.7 |
| Eluent: | 3.2 mM sodium carbonate, |
| | 1.0 mM sodium hydrogen carbonate in water |
| Type of method | Cation exchange |
| Column: | Metrosep C 2-250 (4.0 × 250 mm) |
| Column Temperature (° C.): | Ambient |
| Injection (μl): | 20 |
| Detection: | Conductivity detector |
| Flow Rate (ml · min⁻¹): | 1.0 |
| Eluent: | 4.0 mM Tartaric acid, |
| | 0.75 mM Dipicolinic acid in water |

### Example 1

### Synthesis of the Intermediate Sulfonylurea Carbamate (8)

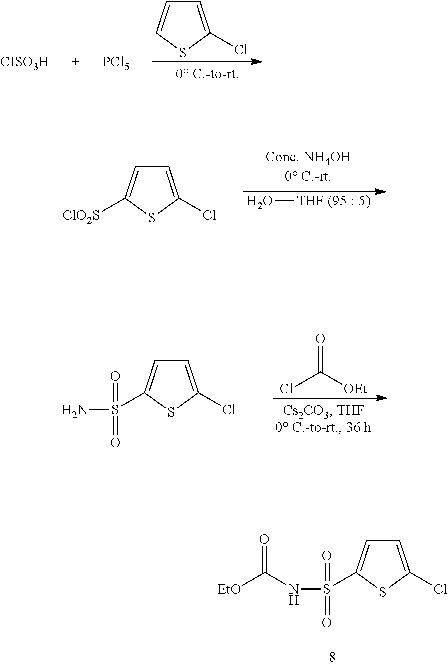

### Step 1—Preparation 5-chlorothiophene-2-sulfonyl chloride

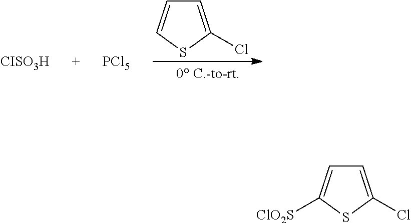

The following procedure was adapted from C. A. Hunt, et al. J. Med. Chem. 1994, 37, 240-247. In a three-necked R.B. flask, equipped with a mechanical stirrer, an air condenser, a dropping funnel, and a moisture-guard tube, was placed chlorosulfonic acid (240 mL, 3.594 mol). Under stirring, PCl₅ (300 g, 1.44 mol, 0.40 equiv) was added in portions, over ca. 45 mins. During the addition, a large volume of HCl gas evolved vigorously, but the temperature of the mixture did not rise significantly (<40° C.). By the time all the PCl₅ had been added, an almost clear, pale yellow solution resulted, with only a few solid pieces of PCl₅ floating in the suspension. It was stirred until gas evolution ceased (0.5 h).

Then the reaction vessel was cooled in ice, and 2-chloro-thiophene (66.0 mL, 0.715 mol) was added via the dropping funnel, over 1.0 h. With the addition of the very first few drops of 2-Cl-thiophene, the mixture turned dark purple, and by the time all of the thiophene had been added, a dark purple solution resulted. During the addition, HCl gas evolved continuously, at a slow rate. The reaction mixture was then stirred at room temperature overnight.

Then the mixture, dark-purple clear solution, was added dropwise to crushed ice (3 L), over 0.5 h. On addition to ice, the purple color disappeared instantaneously; the colorless thin emulsion was stirred mechanically at room temperature for ca. 15 h. Then the mixture was extracted with CH₂Cl₂ (3×300 mL). The combined CH₂Cl₂-extract was washed with water (1×200 mL), saturated NaHCO₃ (1×250 mL), brine (1×100 mL), dried (Na₂SO₄), and concentrated on a rotary evaporator to yield the crude product as a pale yellow glue, which showed a tendency to solidify, yielding a semi-solid mass. This was then purified by high-vacuum distillation (bp 110-112°/12 mm) to yield 135.20 g (88%) of the title compound as a colorless/pale-yellow semi solid.

### Step 2—5-chlorothiophene-2-sulfonamide

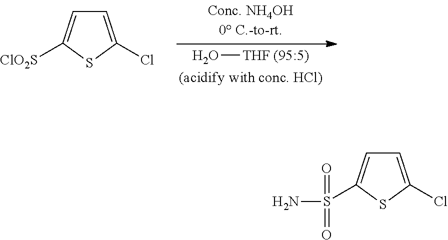

The following procedure was adapted from C. A. Hunt, et al. J. Med. Chem. 1994, 37, 240-247. In a three-necked R.B. flask, equipped with a mechanical stirrer, conc. NH₄OH (500 mL, 148.50 g NH₃, 8.735 mol NH₃, 13.07 equiv NH₃) was placed. The flask was cooled in ice and 5-chlorothiophene-2-sulfonyl chloride (145.0 g, 0.668 mol) was added, in portions over 0.5 h (it is a low-melting solid, and it was melted by warming, which was then conveniently added via a wide-bored polyethylene pipette). The sulfonyl chloride immediately solidifies in the reaction flask. After all the sulfonyl chloride had been added, the flask containing it was rinsed with THF (25 mL), and this also was transferred to the reaction vessel. Then the heavy suspension was stirred at room temperature for ca. 20 h. At the end of this time the reaction mixture was still a suspension but of a different texture.

Then the mixture was cooled in ice, diluted with H₂O (1.5 l), and acidified with conc. HCl to pH ca. 3. The solid product was collected by filtration using a Buchner funnel, rinsed with cold water, and air-dried to afford the title compound as a white solid, 103.0 g (78%). MS (M−H): 196.0; 198.0

### Step 3—Ethyl 5-chlorothiophen-2-ylsulfonylcarbamate

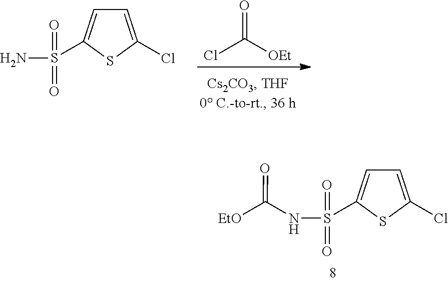

A 2-L 3-necked R.B. flask, equipped with a mechanical stirrer and a dropping funnel, was charged with sulfonamide (60.0 g, 303.79 mmol), and Cs₂CO₃ (200 g, 613.83 mmol, 2.02 equiv) in THF (900 mL). The clear solution was cooled in ice, and ethyl chloroformate (70.0 mL, 734.70 mmol, 2.418 equiv) was added over ca. 30 mins. The heavy suspension was then stirred at room temperature for ca. 36 h.

Then the mixture was diluted with water (200 mL) to yield a clear colorless solution, which was concentrated on rotary evaporator to one-third its volume. This was then diluted with EtOAc (250 mL), cooled in ice, and acidified with 6N HCl to pH ca. 1. The biphasic mixture was transferred to a separatory funnel, layers were separated, and the aqueous layer was again extracted with 2×75 mL EtOAc. The combined organic extract was washed with water/brine (2×50 mL), brine (1×50 mL), dried over Na₂SO₄, and concentrated to yield the title compound as lightly colored oil. This was purified by filtration through a silica-gel plug. The crude product was applied to the silica-gel plug on a sintered funnel in EtOAc, and then was eluted with EtOAc (1 liter). Concentration of the EtOAc filtrate provided the title compound 8 as a white solid, 71.28 g (87%). MS (M−H): 268.0; 270.0. ¹H NMR (DMSO): δ 7.62 (d, 1H), 7.25 (d, 1H), 4.10 (q, 2H), 1.16 (t, 3H).

### Example 2

### Synthesis of [4-(6-fluoro-7-methylamino-2,4-dioxo-1,4-dihydro-2H-quinazolin-3-yl)-phenyl]-5-chloro-thiophen-2-yl-sulfonylurea (7a)

### Step 1

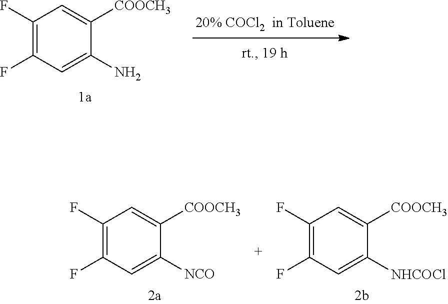

Aniline 1 (¹H NMR (DMSO): δ 7.58 (dd, 1H), 6.72 (dd, 1H), 3.77 (s, 3H); 6.0 g, 32.085 mmol) was placed in a 500 mL round bottomed flask and 20% phosgene in toluene (175 mL, 332.50 mmol, 10.36 equiv) was added. The resulting somewhat sticky suspension was then magnetically stirred overnight at room temperature resulting in a clear, colorless solution. An aliquot removed, blown dry with argon, quenched with MeOH, and analyzed by RP-HPLC/MS to show no unreacted aniline 1 and clean formation of the isocyanate 2a and/or carbamoyl chloride 2b as analyzed as its methyl-carbamate. The mixture was concentrated first by rotary evaporation and then under high vacuum to yield 6.76 g (99% yield) of the isocyanate 2a and/or carbamoyl chloride 2b as a free-flowing white solid.

### Step 2

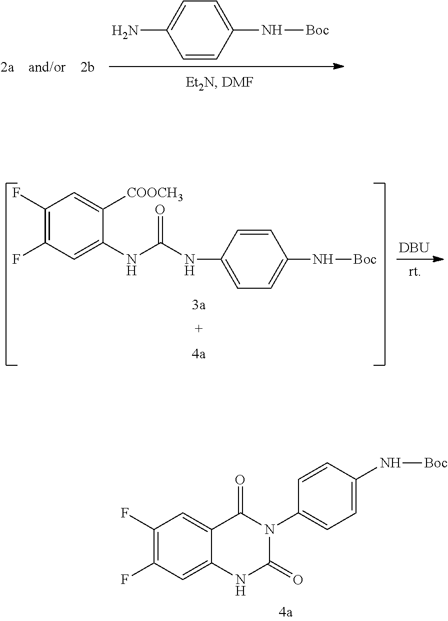

In a 500 mL R.B. flask was placed N-Boc-1,4-phenylenediamine (6.22 g, 29.866 mmol, 1.20 equiv) in DMF (100 mL). Triethylamine (5.30 mL, 38.025 mmol, 1.52 equiv) was syringed in. Then the clear, dark-brown solution was treated with a solution of the isocyanate 2a (5.30 g, 24.88 mmol) and/or carbamoyl chloride 2b in DMF (50 mL), dropwise, over 15 minutes. After the addition was over, a slightly turbid mixture resulted, which was stirred overnight at room-temperature. An aliquot was analyzed, after quenching with MeOH, to show no unreacted isocyanate, and clean formation of the urea, 3a, and quinazoline-1,3-dione, 4a, in a ratio of ca. 2.5:1. MS (M−H): 388.0.

DBU (3.75 mL, 25.07 mmol, ca. 1.0 equiv) was then syringed in, dropwise, over 5 minutes, resulting in a clear dark-brown solution. This was stirred at room temperature for 3.0 h resulting in a turbid mixture. HPLC analysis showed no urea 3a and clean formation of the quinazoline-1,3-dione 4a. The reaction mixture was concentrated on a rotary evaporator to yield the crude product as a solid. This was dried under high vacuum, and then triturated with CH₂Cl₂/H₂O (5:1) to yield 8.40 g of 4a as an almost white solid (87% yield). ¹H NMR (DMSO): δ 9.39 (s, 1H), 7.68 (dd, 1H), 7.45 (d, 2H), 7.03 (m, 2H), 6.98 (dd, 1H), 1.48 (s, 9H).

### Step 3

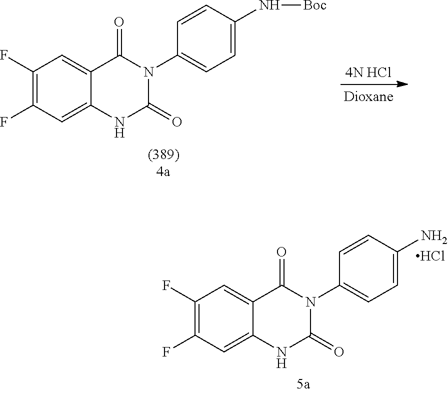

The N-Boc-aniline 4a (4.0 g, 10.28 mmol) was placed in a round-bottomed flask and 4N HCl in dioxane (50.0 mL, 200 mmol, 19.40 equiv) was added. The heavy, negligibly solvated suspension was stirred at room temperature for 5.0 h. HPLC showed no starting material and clean formation of the aniline 5a. The mixture was then concentrated on a rotary evaporator to yield the crude product. The solid thus obtained was triturated with CH₂Cl₂ to yield 3.22 g of pure 5a as an almost white solid (96% yield). MS (M−H): 290.3. ¹H NMR (DMSO): δ 11.75 (s, 1H), 7.88 (dd, 1H), 7.32 (m, 4H), 7.21 (dd, 1H).

### Step 4

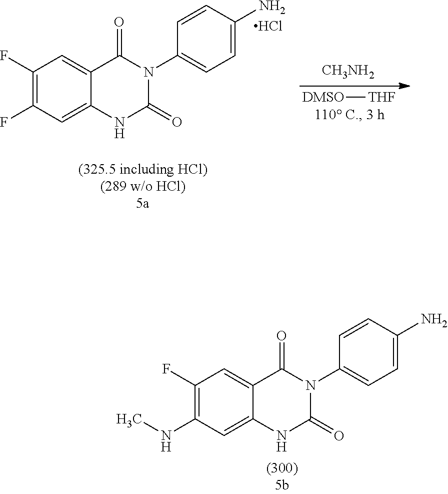

The difluoro-compound, 5a (1.0 g, 3.072 mmol) was placed in a screw-cap sealed tube. DMSO (20 mL) was added, followed by methylamine (2.0M in THF) (15.0 mL, 30 mmol, 9.76 equiv), resulting in a clear solution. This was then heated in an oil bath to 110° C. for 3 h. HPLC showed no unreacted 5a and clean formation of 5b. The mixture was then cooled to room temperature, all the MeNH₂ and THF were evaporated, and the residue was diluted with 100 mL water to precipitate 5b. After stirring for ca. 2 h at room temperature, the white solid was collected by filtration through a Buchner funnel and rinsed with H₂O (100 mL), and air-dried. HPLC analysis of this solid showed it to be pure and devoid of any DBU. This solid was further purified by triturating with Et₂O, and then CH₂Cl₂ as in the previous route to this aniline to give 875 mg of the title compound (95% yield). MS (M+1) 301.2. ¹H NMR (DMSO): δ 11.10 (s, 1H), 7.36 (d, 1H), 6.78 (d, 2H), 6.75 (m, 1H), 6.56 (d, 2H), 6.20 (d, 1H), 5.18 (d, 2H), 2.76 (d, 3H).

### Step 5—Synthesis of 1-(5-chlorothiophen-2-ylsulfonyl)-3-(4-(6-fluoro-7-(methylamino)-2,4-dioxo-1,2-dihydroquinazolin-3(4H)-yl)phenyl)urea (6a)

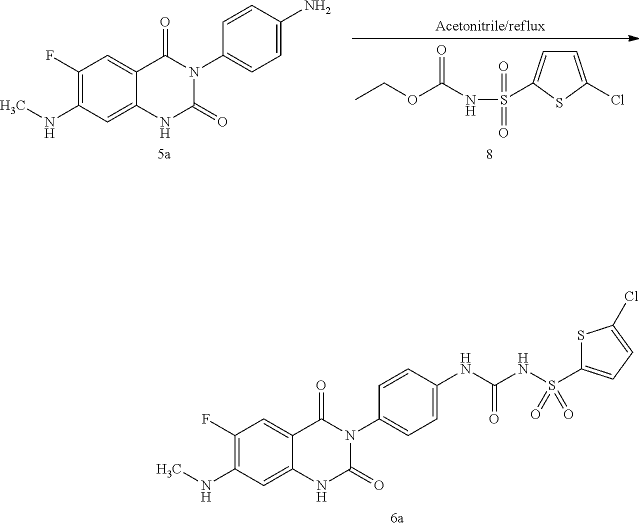

The reaction mixture comprising of the aniline (5a, 16.0 g, 53.33 mmol) and ethyl-sulfonyl-carbamate (8, 28.77 g, 106.66 mmol, 2.0 equiv) in CH₃CN (1300 mL) was heated to reflux for 36 h. During this time, the reaction mixture remained as a heavy suspension. HPLC analysis showed a clean reaction, and <1% unreacted aniline. The heavy suspension was cooled to room temperature and filtered through a Buchner funnel. The white solid product was further rinsed with CH₃CN (3×40 mL). HPLC of the filtrate showed the presence of only a trace amount of the desired product, most of it being the excess carbamate. The crude product was then triturated with CH₂Cl₂ (400 mL), and the almost white solid product (6a) was collected by filtration through a Buchner funnel: Yield, 25.69 g (92%). MS (M+1): 524.0; 526.0. ¹H NMR (DMSO): δ 11.20 (s, 1H), 9.15 (s, 1H), 7.68 (d, 1H), 7.42 (d, 2H), 7.36 (d, 1H), 7.26 (m, 1H), 7.16 (d, 2H), 6.78 (m, 1H), 6.24 (d, 1H), 2.78 (d, 3H).

### Example 3

### [4-(6-chloro-7-methylamino-2,4-dioxo-1,4-dihydro-2H-quinazolin-3-yl)-phenyl]-5-chloro-thiophen-2-yl-sulfonylurea (6b)

The compound in Example 3 is synthesized as described for Example 2 (Step 1-5) except starting with methyl-2-amino-5-chloro-4-fluorobenzoate which was synthesized by reduction of methyl-2-nitro-5-chloro-4-fluorobenzoate with Pt(S)C.

### Example 4

### Synthesis of [4-(6-fluoro-7-methylamino-2,4-dioxo-1,4-dihydro-2H-quinazolin-3-yl)-phenyl]-5-chloro-thiophen-2-yl-sulfonylurea (6a) and potassium salt (7a)

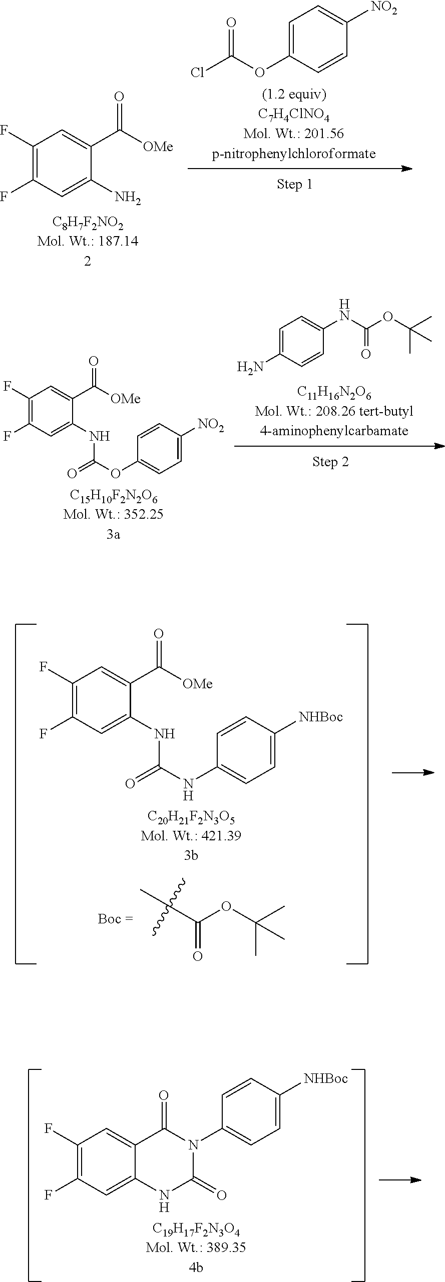

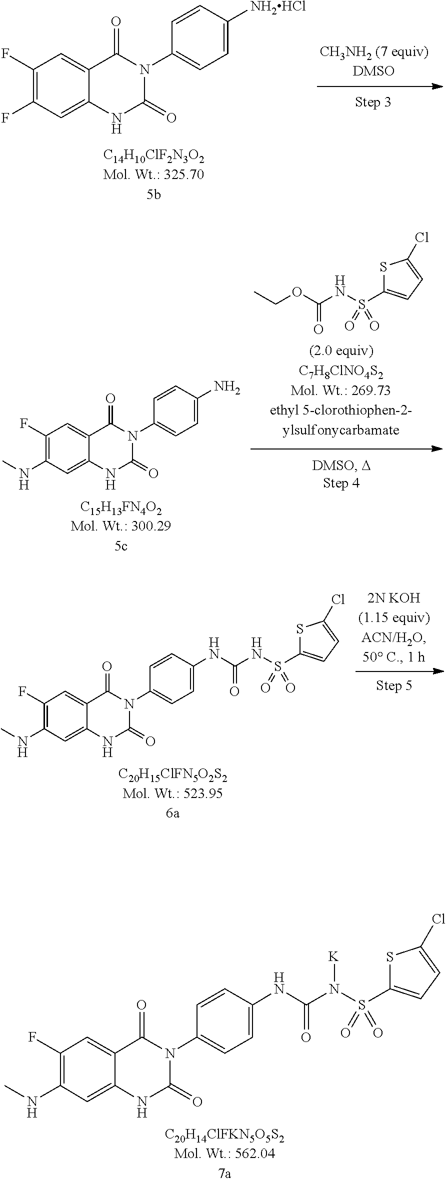

### Step 1

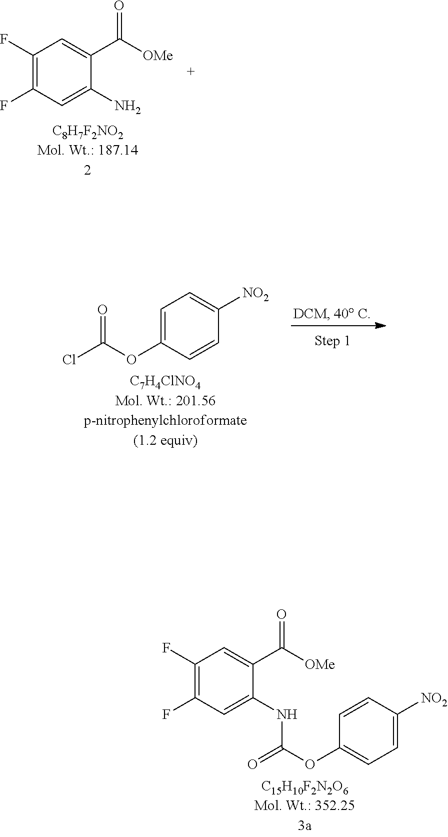

Methyl 2-amino-4,5-difluorobenzoate (2) (38 kg, 1.0 eq) and dichloromethane (560 kg, 8×, ACS>99.5%) were charged to a PP1-R1000 reactor (2000 L GL reactor). The reaction mixture was agitated for 5 mins. 4-Nitrophenylchloroformate (49.1 kg, 1.2 equiv) was charged into PP1-R2000 reactor (200 L) followed by dichloromethane (185 kg) and agitated the contents for 5 mins. After pressurizing the 200 L reactor the 4-nitrophenylchloroformate solution was transferred into the 2000 L reactor containing dichloromethane solution of (2). The reaction mixture was heated to 40±5° C. (reflux) under nitrogen gas purge for 3 hrs. The representative TLC analysis confirmed reaction completion (in-process TLC, no compound (2) remaining; 99:1 CHCl₃-MeOH). The solution was cooled to 30° C. and distilled off 460 kg of dichloromethane under vacuum. The 2000 L reactor was charged with 520 kg of hexanes and cooled the contents of the reactor to 0±5° C. and agitated for 4 hrs. The solid obtained was filtered through GF Nutsche filter lined with a sheet of T-515 LF Typar filter and a sheet of MeI-Tuf 1149-12 filter paper. The filter cake was washed with 20 kg of hexanes and vacuum dried at 35° C. until constant weight attained. The dry product was discharged (70.15 kg) with 98% yield. The product confirmed by ¹H NMR and TLC analysis.

### Step 2. Synthesis of 3-(4-aminophenyl)-6,7-difluoroquinazoline-2,4(1H,3H)-dione hydrochloride, Compound 5b

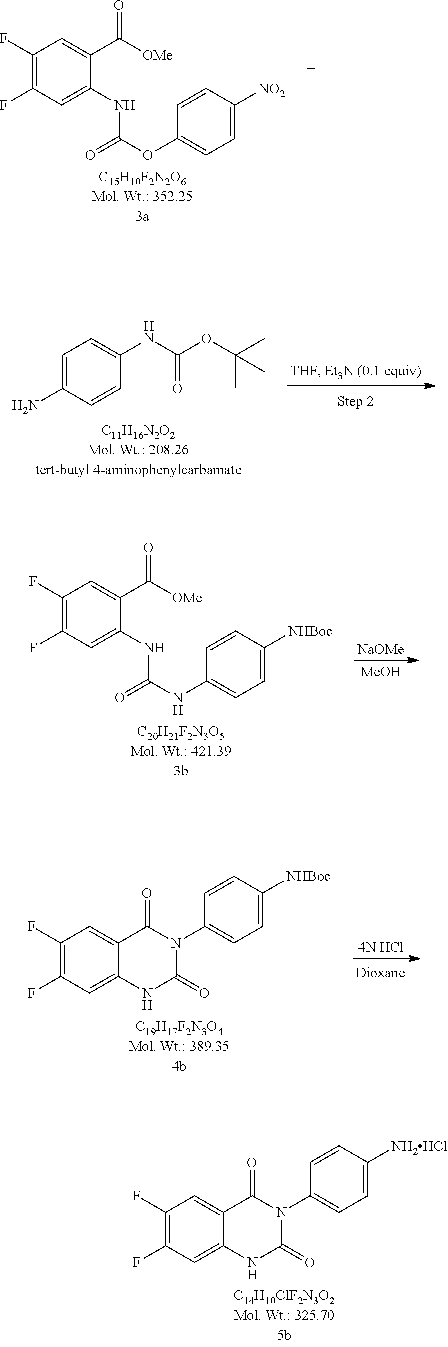

The PP1-R1000 (2000 L GL reactor) reactor was charged with 3a (64.4 kg, 1.0 eq), anhydrous tetrahydrofuran (557 kg) and triethylamine (2.2 kg, 0.1 equiv). The charging line of 2000 L GL reactor was rinsed with tetrahydrofuran (10 kg). The contents of the reactor were agitated for 25 mins during that period complete solution was obtained. The PP1-R2000 (200 L HP reactor) reactor was charged with N-Boc-p-phenylenediamine (38 kg, 1.0 equiv), tetrahydrofuran (89 kg) and agitated for 30 mins until complete solution obtained. The contents of the 200 L HP reactor were transferred to the 2000 L GL reactor containing the compound 3a and then heated at 65±5° C. for 2 hrs. The reaction was deemed complete monitored by HPLC after confirming the disappearance of starting material 3a (in-process specification <1%). The contents of 2000 L GL reactor were cooled to 20±5° C. and then charged with sodium methoxide (25% solution in methanol, 41.5 kg, 1.05 equiv.) over 20 mins. maintaining the temperature below 30° C. The charging lines were rinsed with tetrahydrofuran (10 kg). The contents were agitated at 25±5° C. for 4 hrs. In-process HPLC analysis confirmed the completion of the reaction when the amount of compound 3b remaining in the reaction mixture is <1%. To this reaction mixture added filtered process water (500 kg) and distilled under vacuum the 2000 L GL reactor contents into clean 200 L GL receiver until 300 kg of solvent is distilled. The solids obtained were filtered using GL Nutsche filter and washed with process filtered water until the color of the solid compound 4b is white to grayish. The 2000 L GL reactor is charged with wet compound 4b filter cake, dioxane (340 kg) and agitated the contents for 1 hr. The filterable solid obtained were filtered through GL Nutsche filter with a sheet of T-515 LF Typar filter paper. The solid cake was blow dried for 2 hrs and then charged with dioxane (200 kg) into the 2000 L GL reactor. The contents were agitated for 10 min and then charged with 4 N HCl in dioxane (914 kg) over 3 hrs and maintaining the internal temperature below 30° C. The charging line was rinsed with additional dioxane (10 kg) and the contents of the reactor were agitated for 6 hrs at 25±5° C. The completion of the reaction is monitored by HPLC (in process control compound 4b is <1% in the reaction mixture) for the conversion of compound 4b to compound 5b. The contents of the reactor were cooled to 5±5° C. for 2 hr and the solid obtained was filtered through GL Nutsche filter followed by washing with dioxane (50 kg). The filter cake was blow dried with 8±7 psi of nitrogen for 30 mins. and purity analyzed by HPLC. The filtered solid was dried to constant weight in vacuum oven at 45° C. for 48 hr. The compound 5b (65.8 kg, actual yield 110.6%) was discharged and analyzed by ¹H NMR and HPLC analysis. ¹H NMR (DMSO): δ 11.75 (s, 1H), 7.88 (dd, 1H), 7.32 (m, 4H), 7.21 (dd, 1H).

### Step 3. Synthesis of 3-(4-aminophenyl)-6-fluoro-7-(methylamino)quinazoline-2,4(1H,3H)-dione, Compound 5c

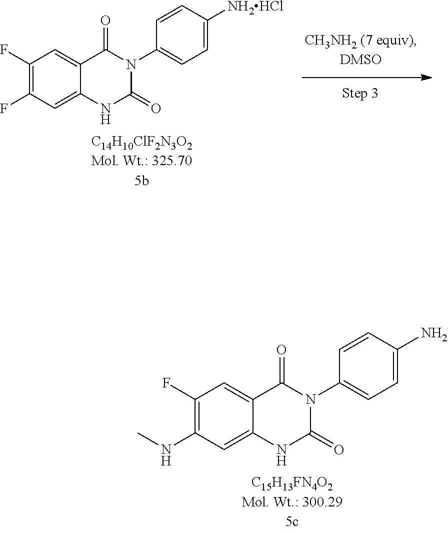

The PP1-R2000 (200 L HP reactor) was charged with compound 5b (18 kg, 1.0 eq.) and pressurized with 100±5 psi of nitrogen. The nitrogen from the reactor was vented through the atmospheric vent line then the condenser valve was opened and the reactor was then charged with dimethyl sulfoxide (>99.7%, 105 kg) under a blanket of argon. The reactor contents were agitated at 22° C. (19-25° C.) for 15 mins and then the maximum achievable vacuum was pulled on the 200 L HP reactor after closing all the remaining valves. Using the established vacuum, methylamine (33% wt % in absolute ethanol, 37.2 kg) was charged to the 200 L HP reactor at a rate that maintained the internal temperature at 25±5° C. while keeping a nitrogen blanket on the reagent solution during the charging. After rinsing the charging line with dimethyl sulfoxide (5 kg) the 200 L HP reactor condenser valve was closed and the reactor contents were heated to 110±5° C. The contents of the reactor were agitated for at least 5 hrs at 110±5° C. In-process HPLC taken after 5 hr 40 mins showed compound 5b content of 0.09%, indicating completion of the reaction (in-process specification ≦1%). The contents of 200 L HP reactor were cooled to 25±5° C. While the 200 L reactor is cooling, all the valves of the PP1-R1000 reactor (2000 L GL reactor) were closed and the reactor was charged with process filtered water (550 kg). The contents of the 200 L HP reactor were transferred to the 2000 L GL reactor over 15 minutes followed by rinsing the charging line with process filtered water (50 kg). The contents of the 2000 L GL reactor were agitated for 2 hrs at 5±5° C. The filterable solids obtained were filtered onto PPF200 (GL nutsche filter) fitted with MeI-Tuf 1149-12 filter paper under vacuum. The wet filter cake was discharged and transferred into pre-lined vacuum trays with Dupont's fluorocarbon film (Kind 100A). The special oven paper (KAVON 992) was clamped down over the vacuum trays containing the wet compound 5c and it was transferred to the vacuum oven tray dryer. The oven temperature was set to 55° C. and compound 5c dried to a constant weight for 12 hrs. The product 5c was discharged (12.70 kg) in 76.5% yield (expected 85-95%). HPLC shows 98.96% purity and ¹H NMR confirmed the structure for compound 5c. ¹H NMR (DMSO): δ 11.10 (s, 1H), 7.36 (d, 1H), 6.78 (d, 2H), 6.75 (m, 1H), 6.56 (d, 2H), 6.20 (d, 1H), 5.18 (d, 2H), 2.76 (d, 3H).

### Step 4. 5-Chloro-N-(4-(6-fluoro-7-(methylamino)-2,4-dioxo-1,2-dihydroquinazolin-3(4H)-yl)phenylcarbamoyl)thiophene-2-sulfonamide

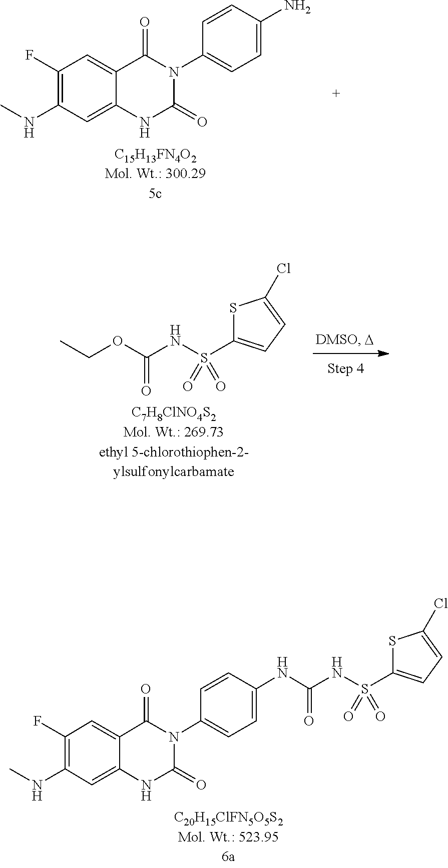

The PP1-R2000 (200 L HP reactor) reactor was charged with 6 (20.7 kg, 1.0 equiv), Ethyl 5-chlorothiophene-2-ylsulfonylcarbamate (37.5 kg, 2.0 equiv, >95%), dimethyl sulfoxide (>99%, 75 kg) and agitated for 15 mins. While pulling maximum achievable vacuum, the 200 L HP reactor Number PP1-R2000 was heated to 65±5° C. for 15 hrs. A representative sample was taken from the reactor for HPLC analysis, in-process HPLC indicated <0.9% compound 5c remaining in the reaction mixture (in-process criteria for reaction completion compound 6a <1%). The 800 L reactor number PP5-R1000 was charged with process filtered water (650 kg) and then the 200 L HP contents were transferred to the 800 L while maintaining the internal temperature below 25° C. The 200 L HP reactor was rinsed with dimethyl sulfoxide (15 kg) and transferred to the 800 L reactor which was then agitated for 2 hrs at 5±5° C. The solid formed was filtered through filter PP-F2000 to a 200 L GL receiver under vacuum and the filter cake was rinsed with process filtered water (60 kg). A representative sample of the wet cake was taken for HPLC analysis, if the purity of compound 6a is <95% (in-process control <95%) then dichloromethane trituration is needed). The 800 L GL reactor was charged with all the wet compound 6a, dichloromethane (315 kg) and the contents were agitated for 3 hrs. The solid was filtered through GL nutsche filter lined with 1 sheet of T515 LF TYPAR filter under vacuum. The filter cake was washed with dichloromethane (50 kg) and the cake was blow dried with 8±7 psi of nitrogen for 15 mins. The filter cake was transferred into pre-lined vacuum trays with Dupont fluorocarbon film (Kind 100A) and then put into the vacuum oven tray dryer set at 60° C. for 12 hrs. The dried compound 6a was isolated (33.6 kg, 93% yield) with HPLC purity of 93.5% and 4.3% of sulfonamide. ¹H NMR confirmed the structure for compound 6a. ¹H NMR (DMSO): δ 11.20 (s, 1H), 9.15 (s, 1H), 7.68 (d, 1H), 7.42 (d, 2H), 7.36 (d, 1H), 7.26 (m, 1H), 7.16 (d, 2H), 6.78 (m, 1H), 6.24 (d, 1H), 2.78 (d, 3H).

### Step 5. Potassium (5-chlorothiophen-2-ylsulfonyl)(4-(6-fluoro-7-(methylamino)-2,4-dioxo-1,2-dihydroquinazolin-3(4H)-yl)phenylcarbamoyl)amide, 7a

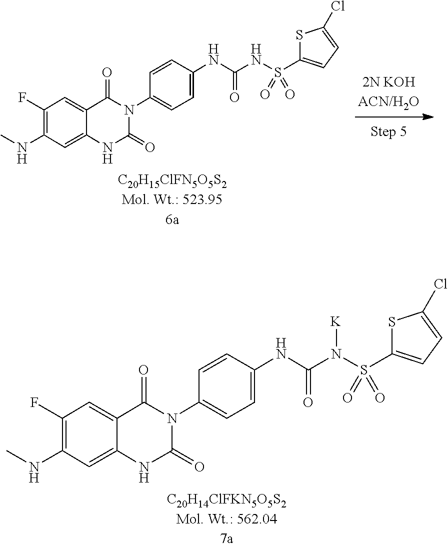

The 800 L GL reactor number PP5-R1000 was charged with acetonitrile (134 kg), WFI quality water (156 kg) and the contents were agitated for 5 mins. To this, compound 6a (33.6 kg, 1.0 equiv) was added and the reaction mixture was a suspension at this point. The suspension was charged with aqueous solution (WFI water, 35 kg) of potassium hydroxide (4.14 kg, 1.15 equiv, >85%) at a rate that maintains the internal temperature below 30° C. The charging lines were rinsed with WFI quality water (2 kg) followed by heating the 800 L GL reactor contents to 50±5° C. for 1 hr. The contents were then filtered hot through a bag filter, then a seven cartridge 0.2 μm polish filter to clean the HDPE drums. The hot filtration system was maintained through out the filtration process so no material crashed out of the solution. The 800 L GL reactor jacket was cooled to 25±5° C. before proceeding to the reactor rinse. The 800 L GL reactor was rinsed with a pre-mixed solution of acetonitrile (8.5 kg) and WFI quality water (10 kg) through the filter system into the drums labeled as 7a hot filtration. Using the pressure vessel the 800 L GL reactor was rinsed with WFI quality water (20 kg) followed by acetone (20 kg) then blown dry with nitrogen (3±2 psi). The 800GL reactor bottom valve was closed and 20±10 inches Hg of vacuum was pulled. The vacuum was broken and the reactor charged with the contents of the drums labeled as 7a hot filtration. The 800 L GL reactor number PP5-R1000 contents was cooled to 20±5° C. and then, using a polish filter (PP-PF09), the reactor was charged with methanol (373 kg, >99%) maintaining the internal temperature below 30° C. The contents of the 800GL reactor number PP5-R1000 were cooled to 15±5° C. followed by agitation of the contents for 12 hrs at this temperature. During this time the filterable solids were filtered through a clean filter apparatus (PP-F1000) into clean 200 L GL receiver (PPR-04) followed by pressurization of the reactor. 20±10 inches Hg of vacuum was pulled on the filter/receiver and the contents were filtered. The filter cake was washed with methanol (30 kg) and blown dry with 8±7 psi of nitrogen for 10 mins. The vacuum oven tray dryer temperature was set to 80° C. prior to loading the wet cake of 7a. The wet filter cake was transferred into the pre-lined vacuum trays with Dupont's fluorocarbon film—Kind 100A and the special oven paper (Kavon MeI Tuf paper) was clamped down over the vacuum trays containing the wet product 7a. The trays were transferred to the vacuum oven tray dryer. The wet 7a was dried to a constant weight (constant weight is defined as tray reading at least 1 hr apart having the same weight within ±50 g. The representative sample was analyzed for residual solvents (residual solvent specifications for API) and it met the specifications. The final API was subjected to equilibration with water (5-6%) for 12 hrs with a tray of WFI quality water present, then thoroughly turned and allowed to stand for an additional 12 hrs and finally subjected to KF analysis (5.5% water content). Compound 7 potassium salt (21.80 kg, 60.6% yield) was transferred to double heavy-duty poly bags and stored in secondary containment. HPLC showed a purity of 99.7% for 7a and ¹H NMR confirmed the structure for 7a. ¹H NMR (DMSO): δ 11.14 (s, 1H), 8.60 (s, 1H), 7.48 (m, 2H), 7.35 (d, 1H), 7.22 (d, 1H), 6.95 (m, 3H), 6.75 (m, 1H), 6.22 (d, 1H), 2.78 (d, 3H).

### Example 5

### Pharmacological Assays

The pharmacological activity of each of the compounds according to the invention is determined by the following in vitro assays:

### I. Inhibition of ADP-Mediated Platelet Aggregation In Vitro

1.

The effect of testing the compound according to the invention on ADP-induced human platelet aggregation was assessed in a 96-well microtiter assay (see generally the procedures in Jantzen, H. M. et al. (1999) Thromb. Hemost. 81:111-117) or standard cuvette light transmittance aggregometry using either human platelet-rich plasma (PRP) or human washed platelets.

For preparation of human platelet-rich plasma for aggregation assays, human venous blood was collected from healthy, drug-free volunteers into 0.38% sodium citrate (0.013 M, pH 7.0 final). Platelet-rich plasma (PRP) is prepared by centrifugation of whole blood at 160×g for 20 minutes at room temperature. The PRP layer is removed, transferred to a new tube, and the platelet count is adjusted, if necessary, to achieve a platelet concentration of ˜3×10⁸ platelets/ml using platelet-poor plasma (PPP). PPP is prepared by centrifugation of the remaining blood sample (after removal of PRP) for 20 minutes at 800×g. This preparation of PRP can subsequently be used for aggregation assays in either a 96-well plate or standard cuvette aggregometry.

For preparation of washed platelets, human venous blood is collected from healthy, drug-free volunteers into ACD (85 mM sodium citrate, 111 mM glucose, 71.4 mM citric acid) containing PGI₂ (1.25 ml ACD containing 0.2 μM PGI2 final; PGI₂ was from Sigma, St. Louis, Mo.). Platelet-rich plasma (PRP) is prepared by centrifugation at 160×g for 20 minutes at room temperature. Washed platelets are prepared by centrifuging PRP for 10 minutes at 730×g and re-suspending the platelet pellet in CGS (13 mM sodium citrate, 30 mM glucose, 120 mM NaCl; 2 ml CGS/10 ml original blood volume) containing 1 U/ml apyrase (grade V, Sigma, St. Louis, Mo.). After incubation at 37° C. for 15 minutes, the platelets are collected by centrifugation at 730×g for 10 minutes and re-suspended at a concentration of 3×10⁸ platelets/ml in Hepes-Tyrode's buffer (10 mM Hepes, 138 mM NaCl, 5.5 mM glucose, 2.9 mM KCl, 12 mM NaHCO₃, pH 7.4) containing 0.1% bovine serum albumin, 1 mM CaCl₂ and 1 mM MgCl₂. This platelet suspension is kept >45 minutes at 37° C. before use in aggregation assays.

2.

For cuvette light transmittance aggregation assays, serial dilutions (1:3) of test compounds were prepared in 100% DMSO in a 96 well V-bottom plate (final DMSO concentration in the cuvette was 0.6%). The test compound (3 μl of serial dilutions in DMSO) was pre-incubated with PRP for 30-45 seconds prior to initiation of aggregation reactions, which were performed in a ChronoLog aggregometer by addition of agonist (5 or 10 μM ADP) to 490 μL of PRP at 37° C. In some cases, light transmittance aggregometry was performed using 490 μL of washed platelets (prepared as described above) at 37° C., and aggregation was initiated by addition of 5 μM ADP and 0.5 mg/ml human fibrinogen (American Diagnostics, Inc., Greenwich, Conn.). The aggregation reaction is recorded for ˜5 mins, and maximum extent of aggregation is determined by the difference in extent of aggregation at baseline, compared to the maximum aggregation that occurs during the five minute period of the assay. Inhibition of aggregation was calculated as the maximum aggregation observed in the presence of inhibitor, compared to that in the absence of inhibitor. IC₅₀ values were derived by non-linear regression analysis using the Prism software (GraphPad, San Diego, Calif.).

3.

Inhibition of ADP-dependent aggregation was also determined in 96-well flat-bottom microtiter plates using a microtiter plate shaker and plate reader similar to the procedure described by Frantantoni et al., Am. J. Clin. Pathol. 94, 613 (1990). All steps are performed at room temperature. For 96-well plate aggregation using platelet-rich plasma (PRP), the total reaction volume of 0.2 ml/well includes 180 μl of PRP (˜3×108 platelets/ml, see above), 6 μl of either serial dilution of test compounds in 20% DMSO or buffer (for control wells), and 10 μl of 20×ADP agonist solution (100 μM). The OD of the samples is then determined at 450 nm using a microtiter plate reader (Softmax, Molecular Devices, Menlo Park, Calif.) resulting in the 0 minute reading. The plates are then agitated for 5 min on a microtiter plate shaker and the 5 minute reading is obtained in the plate reader. Aggregation is calculated from the decrease of OD at 450 nm at t=5 minutes compared to t=0 minutes and is expressed as % of the decrease in the ADP control samples after correcting for changes in the unaggregated control samples. IC₅₀ values were derived by non-linear regression analysis.

For 96-well plate aggregation using washed platelets, the total reaction volume of 0.2 ml/well includes in Hepes-Tyrodes buffer/0.1% BSA: 4.5×10⁷ apyrase-washed platelets, 0.5 mg/ml human fibrinogen (American Diagnostica, Inc., Greenwich, Conn.), serial dilutions of test compounds (buffer for control wells) in 0.6% DMSO. After ˜5 minutes pre-incubation at room temperature, ADP is added to a final concentration of 2 μM which induces submaximal aggregation. Buffer is added instead of ADP to one set of control wells (ADP− control). The OD of the samples is then determined at 450 nm using a microtiter plate reader (Softmax, Molecular Devices, Menlo Park, Calif.) resulting in the 0 minute reading. The plates are then agitated for 5 min on a microtiter plate shaker and the 5 minute reading is obtained in the plate reader. Aggregation is calculated from the decrease of OD at 450 nm at t=5 minutes compared to t=0 minutes and is expressed as % of the decrease in the ADP control samples after correcting for changes in the unaggregated control samples. IC₅₀ values were derived by non-linear regression analysis.

### II. Inhibition of [3H]2-MeS-ADP Binding to Platelets

### 1. The Ability of Candidate Molecules to Inhibit the Binding of [3H]2-MeS-ADP to the P2Y12 Receptor on Platelets was Determined Using a Radioligand Binding Assay.

Utilizing this assay the potency of inhibition of such compounds with respect to [³H]2-MeS-ADP binding to whole platelets is determined. Under the conditions described in II (3) below, the binding of [³H]2-MeS-ADP is solely due to the interaction of this ligand with the P2Y₁₂ receptor, in that all the specific binding measured in this assay is competable with a P2Y₁₂ antagonist (i.e., the specific binding is reduced to background levels by competition with an excess of P2Y₁₂ antagonist, with no competition of binding when a P2Y₁ antagonist is pre-incubated with the platelet preparation). [³H]2-MeS-ADP binding experiments are routinely performed with outdated human platelets collected by standard procedures at hospital blood banks. Apyrase-washed outdated platelets are prepared as follows (all steps at room temperature, if not indicated otherwise):

Outdated platelet suspensions are diluted with 1 volume of CGS and platelets pelleted by centrifugation at 1900×g for 45 minutes. Platelet pellets are re-suspended at 3-6×10⁹ platelets/ml in CGS containing 1 U/ml apyrase (grade V, Sigma, St. Louis, Mo.) and incubated for 15 minutes at 37° C. After centrifugation at 730×g for 20 minutes, pellets are re-suspended in Hepes-Tyrode's buffer containing 0.1% BSA (Sigma, St. Louis, Mo.) at a concentration of 6.66×10⁸ platelets/ml. Binding experiments are performed after >45 minutes resting of the platelets.

2.

Alternatively, binding experiments are performed with fresh human platelets prepared as described in section I (Inhibition of ADP-Mediated Platelet Aggregation in vitro), except that platelets are re-suspended in Hepes-Tyrode's buffer containing 0.1% BSA (Sigma, St. Louis, Mo.) at a concentration of 6.66×10⁸ platelets/ml. Very similar results are obtained with fresh and outdated platelets.

3.

A platelet ADP receptor binding assay (ARB) using the tritiated potent agonist ligand [³H]2-MeS-ADP (Jantzen, H. M. et al. (1999) Thromb. Hemost. 81:111-117) has been adapted to the 96-well microtiter format. In an assay volume of 0.2 ml Hepes-Tyrode's buffer with 0.1% BSA and 0.6% DMSO, 1×10⁸ apyrase-washed platelets are pre-incubated in 96-well flat bottom microtiter plates for 5 minutes with serial dilutions of test compounds before addition of 1 nM [³H]2-MeS-ADP ([³H]2-methylthioadenosine-5′-diphosphate, ammonium salt; specific activity 20-50 Ci/mmole, obtained by custom synthesis from Amersham Life Science, Inc., Arlington Heights, Ill., or NEN Life Science Products, Boston, Mass.). Total binding is determined in the absence of test compounds. Samples for nonspecific binding may contain 10 μM unlabelled 2-MeS-ADP (RBI, Natick, Mass.). After incubation for 15 minutes at room temperature, unbound radioligand is separated by rapid filtration and two washes with cold (4-8° C.) Binding Wash Buffer (10 mM Hepes pH 7.4, 138 mM NaCl) using a 96-well cell harvester (Minidisc 96, Skatron Instruments, Sterling, Va.) and 8×12 GF/C glassfiber filtermats (Printed Filtermat A, for 1450 Microbeta, Wallac Inc., Gaithersburg, Md.). The platelet-bound radioactivity on the filtermats is determined in a scintillation counter (Microbeta 1450, Wallac Inc., Gaithersburg, Md.). Specific binding is determined by subtraction of non-specific binding from total binding, and specific binding in the presence of test compounds is expressed as % of specific binding in the absence of test compound dilutions. IC₅₀ values were derived by non-linear regression analysis.

In the table below, activity in the PRP assay is provided as follows: +++, IC₅₀<10 μM; ++, 10 μM<IC₅₀<30 μM. Activity in the ARB assay is provided as follows: +++, IC₅₀<0.05 μM; ++, 0.05 μM<IC₅₀<0.5 μM.

TABLE 3

| Example No. | ARB Binding | PRP Activity |
|---|---|---|
| Example 2 | +++ | +++ |
| Example 3 | + | + |

### Example 6

### Synthesis of [4-(6-fluoro-7-methylamino-2,4-dioxo-1,4-dihydro-2H-quinazolin-3-yl)-phenyl]-5-chloro-thiophen-2-yl-sulfonylurea potassium salt (9a) (Amorphous Form)

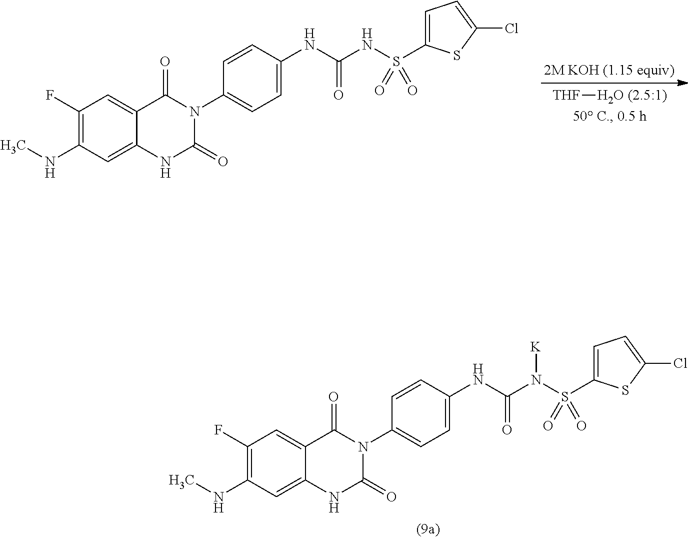

The free-acid, sulfonylurea, (7.0 g, 13.365 mmol) was suspended in THF/H₂O (55:22 mL, ca. 2.5:1), and treated with 2M KOH (7.70 mL, 15.40 mmol, 1.15 equiv) drop wise, over ca. 5 min. By the time the addition was over, a clear solution resulted. However, a solid precipitated out after <5 mins and the reaction mixture became a heavy suspension. This was heated in an oil-bath to 50° C., and the resulting clear viscous light brown solution was held at this temperature for 0.5 h. On cooling to rt., the title compound (9a) precipitated out. The mixture was diluted with i-PrOH (250 mL, 3× the original reaction volume), stirred at rt. for 3 h, and then filtered through a Buchner funnel to yield the title compound (9a) as a white solid. This was dried in a vacuum oven at 80° C. to yield 7.20 g (96%) of an amorphous solid. MS (negative scan): 521.7; 523.7.

### Example 7

### Conversion of the Sulfonylurea (7a) to its Amorphous Sodium Salt (10a)

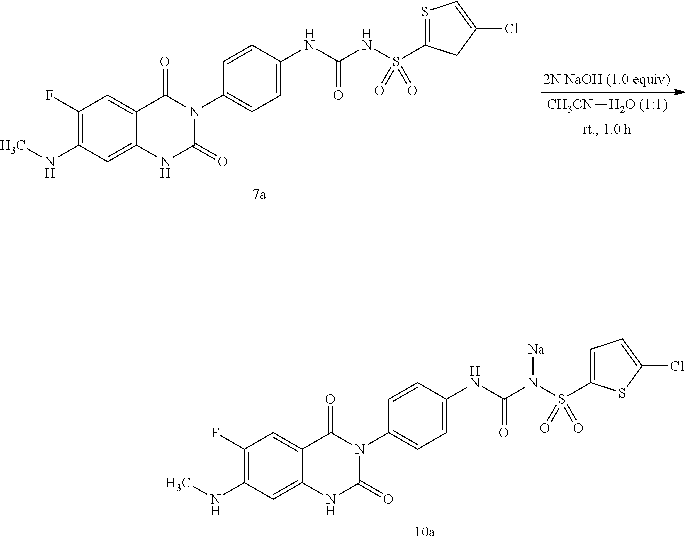

1-(5-chlorothiophen-2-yl sulfonyl)-3-(4-(6-fluoro-7-(methylamino)-2,4-dioxo-1,2-dihydroquinazolin-3(4H)-yl)phenyl)urea (3.0 g, 5.728 mmol) 7a was suspended in CH₃CN/H₂O) (1:1; 70 mL) and was treated with 2N NaOH (2.90 mL, 5.80 mmol), dropwise. Within ca. 15 minutes, a clear solution resulted. After stirring for 1.0 h, the now light brown solution was lyophilized to afford the crude product as an amorphous solid 10a. MS (negative scan): 522.0; 524.0.

### Example 8

### Alternative Preparation of Amorphous Form of the Sodium Salt

Sodium salt 10a was suspended in isopropanol (100 mL) and refluxed for ca. 45 min, then hot filtered to yield a tan coloured solid, which is mostly the title compound by HPLC. The solid was suspended in CH₃CN:EtOH (1:2) (100 mL) and refluxed for 45 mins, then hot filtered to afford 2.54 g of the title compound 10a as a tan coloured solid (99.7% pure by analytical HPLC, long column). The filtrate was diluted with EtOH until the ratio of ACN:EtOH became (1:3) and it was let to stand at room temperature overnight. An additional crop of the title compound precipitated out to afford 210 mg of solid 10a (purity: 99.7% by analytical HPLC, long column).

### Example 9

### Salt Screen of [4-(6-fluoro-7-methylamino-2,4-dioxo-1,4-dihydro-2H-quinazolin-3-yl)-phenyl]-5-chloro-thiophen-2-yl-sulfonylurea

### Primary Screen

To 20 mg of [4-(6-fluoro-7-methylamino-2,4-dioxo-1,4-dihydro-2H-quinazolin-3-yl)-phenyl]-5-chloro-thiophen-2-yl-sulfonylurea in 3 mL of the various solvents, was added 1.1 eq. of the base in 1 mL solvent. The mixture was shaken for 2 hours and the solutions were left to evaporate down to half their volume to try to precipitate out the salt. The results are presented in Table 4 below, which shows the bases used for the screen. The solutions in THF evaporated down to solids very quickly and these were analysed by XRPD. Most samples from THF were amorphous oily solids which were left to maturate at 50° C./ambient temperature. Any solutions that did not form a solid by evaporation had IPA added as an anti-solvent to induce solid to precipitate. Samples with IPA that did not precipitate were left to evaporate. As shown in Table 4 below, the solutions yielded some solids and some oils. Oils/emulsions and opaque liquids were left to maturate at 50° C./ambient in an 8 hour cycles for several weeks. Microscopy and XRPD results showed some samples were crystalline but lack of a solid meant clear diffractograms could not be obtained. Solid samples (crystalline and amorphous) were then filtered, dried and then analyzed to judge their purity, crystallinity and stability. Solids were analysed by ¹H NMR to confirm salt formation and analyzed by ion chromatography and TGA to obtain the stoichiometry of the salt.

TABLE 4
Primary Salt Screen

| | Solvent | | | | |
|---|---|---|---|---|---|
| Base | MeCN/Water | IPA | Water | DMSO | THF |
| Potassium hydroxide | solution | Partially crystalline | Partially crystalline | solution | Partially crystalline |
| | | not acid or base | not acid or base | | not acid or base |
| Sodium hydroxide | solution | Partially crystalline | solution | solution | Partially crystalline |
| | | not acid or base | | | not acid or base |
| Calcium acetate | Partially crystalline | Partially crystalline | Weakly crystalline | solution | emulsion |
| | matches free acid | not acid or base | possibly free acid | | |
| L-lysine monohydrate | solution | Amorphous | Amorphous | solution | oil |
| Ammonium hydroxide | solution | Partially crystalline | Amorphous | solution | Partially crystalline |
| | | not acid or base | | | not acid or base |
| Magnesium acetate | Partially crystalline | Partially crystalline | Partially crystalline | solution | Partially crystalline |
| | matches free acid | not acid or base | matches free acid | | matches free acid |
| L-arginine | oil | Amorphous | Amorphous | solution | oil |
| Tromethamine | Amorphous | Amorphous | Partially crystalline | solution | oil |
| | | | matches free acid | | |
| N-ethylglucamine | Amorphous | solution | Partially crystalline | solution | oil |
| | | | not acid or base | | |
| N-methylglucamine | solution | gel | Amorphous | solution | oil |
| Potassium ethoxide | solution (some | Amorphous | Amorphous | solution | Weakly crystalline |
| | ppt) | | | | possibly free acid |
| Sodium ethoxide | Amorphous | Partially crystalline | Amorphous | solution | Partially crystalline |
| | | not acid or base | | | not acid or base |

TABLES 5a and 5b
Characterisation results
Table 5a-part 1

| | | Physical | |
|---|---|---|---|
| Cation | Solvent | state | XRPD of the dried sample |
| Potassium hydroxide | MeCN/H₂O | solid | partially crystalline, Form B |
| Potassium ethoxide | MeCN/H₂O | solid | similar to Form B |
| Potassium hydroxide | IPA | solid | partially crystalline, Form B |
| Potassium ethoxide | IPA | solid | partially crystalline, Form B |
| Potassium hydroxide | water | solid | partially crystalline, Form C |
| Potassium ethoxide | water | solid | crystalline, Form C |
| Potassium hydroxide | DMSO | solution | insufficient solid |
| Potassium ethoxide | DMSO | solution | insufficient solid |
| Potassium hydroxide | THF | solid | weakly crystalline, Form D |
| Potassium ethoxide | THF | solid | weakly crystalline, Form D |
| Sodium hydroxide | MeCN/H₂O | oil | evaporated to an oil |
| Sodium ethoxide | MeCN/H₂O | solid | matches free acid |
| Sodium hydroxide | IPA | solid | weakly crystalline, Form A |
| Sodium ethoxide | IPA | solid | weakly crystalline, Form B |
| Sodium hydroxide | water | oil | evaporated to an oil |
| Sodium ethoxide | water | solid | matches free acid |
| Sodium hydroxide | DMSO | solution | insufficient solid |
| Sodium ethoxide | DMSO | solution | insufficient solid |
| Sodium hydroxide | THF | solid | weakly crystalline, Form A |
| Sodium ethoxide | THF | solid | weakly crystalline, Form A |
| Calcium acetate | MeCN/H₂O | solid | matches free acid |
| Calcium acetate | IPA | solid | crystalline, Form A |
| Calcium acetate | water | solid | matches free acid |
| Calcium acetate | DMSO | solution | insufficient solid |
| Calcium acetate | THF | emulsion | partially crystalline, Form B |
| L-lysine monohydrate | MeCN/H₂O | oil | evaporated to an oil |
| L-lysine monohydrate | IPA | solid | weakly crystalline, Form A |
| L-lysine monohydrate | water | solid | matches free acid |
| L-lysine monohydrate | DMSO | solution | insufficient solid |
| L-lysine monohydrate | THF | oil | evaporated to a gum |

Table 5a-part

| | | | IC | TGA | Adjusted |
|---|---|---|---|---|---|
| Cation | Solvent | ¹H NMR | acid:base | solvent | IC result |
| Potassium hydroxide | MeCN/H₂O | shifts seen, water and IPA | 1:1.00 | 0% | 1:1.00 |
| Potassium ethoxide | MeCN/H₂O | shifts seen, water and IPA | 1:0.96 | 3.3% | 1:0.99 |
| Potassium hydroxide | IPA | shifts seen, water and IPA | 1:1.16 | 1.5% | 1:1.18 |
| Potassium ethoxide | IPA | shifts seen, water and IPA | 1:1.01 | 3.2% | 1:1.04 |
| Potassium hydroxide | water | shifts seen, water | 1:0.87 | 9.7% | 1:0.96 |
| Potassium ethoxide | water | shifts seen, water | 1:0.92 | 9.5% | 1:1.01 |
| Potassium hydroxide | DMSO | n/a | n/a | n/a | n/a |
| Potassium ethoxide | DMSO | n/a | n/a | n/a | n/a |
| Potassium hydroxide | THF | shifts seen THF, water, | 1:0.86 | 7.1% | 1:0.92 |
| | | DMF | | | |
| Potassium ethoxide | THF | shifts seen, THF, DMF, | 1:0.82 | 8.2% | 1:0.89 |
| | | IPA and water | | | |
| Sodium hydroxide | MeCN/H₂O | n/a | n/a | n/a | n/a |
| Sodium ethoxide | MeCN/H₂O | n/a | n/a | n/a | n/a |
| Sodium hydroxide | IPA | shifts seen, IPA, water | 1:0.92 | 3.3% | 1:0.95 |
| | | (trace THF, DMF) | | | |
| Sodium ethoxide | IPA | shifts seen IPA, water, | 1:0.8 | 8.5% | 1:0.88 |
| | | DMF | | | |
| Sodium hydroxide | water | n/a | n/a | n/a | n/a |
| Sodium ethoxide | water | n/a | n/a | n/a | n/a |
| Sodium hydroxide | DMSO | n/a | n/a | n/a | n/a |
| Sodium ethoxide | DMSO | n/a | n/a | n/a | n/a |
| Sodium hydroxide | THF | shifts seen, THF, water, | 1:0.95 | 4.9% | 1:1.0 |
| | | IPA | | | |
| Sodium ethoxide | THF | shifts seen THF, water, | 1:0.89 | 7.8% | 1:0.96 |
| | | DMF | | | |
| Calcium acetate | MeCN/H₂O | n/a | n/a | n/a | n/a |
| Calcium acetate | IPA | shifts seen, IPA, water | 1:0.9 | 9.1% | 1:0.99 |
| | | (trace THF) | | | |
| Calcium acetate | water | no shifts seen, free acid | n/a | n/a | n/a |
| Calcium acetate | DMSO | n/a | n/a | n/a | n/a |
| Calcium acetate | THF | shifts seen, water and THF | 1:1.16 | 2.1% | 1:1.18 |
| L-lysine monohydrate | MeCN/H₂O | n/a | n/a | n/a | n/a |
| L-Iysine monohydrate | IPA | shifts seen, water and IPA | 1:0.77 | 7.3% | 1:0.83 |
| L-lysine monohydrate | water | n/a | n/a | n/a | n/a |
| L-lysine monohydrate | DMSO | n/a | n/a | n/a | n/a |
| L-lysine monohydrate | THF | n/a | n/a | n/a | n/a |

Table 5b-part 1

Physical

| Cation | Solvent | state | XRPD of the dried sample |
|---|---|---|---|
| Ammonium hydroxide | MeCN/H₂O | solid | crystalline, Form B |
| Ammonium hydroxide | IPA | solid | partially crystalline, Form A |
| Ammonium hydroxide | water | solid | crystalline, Form B |
| Ammonium hydroxide | DMSO | solution | insufficient solid |
| Ammonium hydroxide | THF | solid | weakly crystalline, Form A |
| Magnesium acetate | MeCN/H₂O | solid | matches free acid |
| Magnesium acetate | IPA | solid | partially crystalline, Form A |
| Magnesium acetate | water | solid | matches free acid |
| Magnesium acetate | DMSO | solution | insufficient solid |
| Magnesium acetate | THF | solid | mixture of free acid and base |
| L-arginine | MeCN/H₂O | oil | amorphous |
| L-arginine | IPA | solid | amorphous |
| L-arginine | water | solid | amorphous |
| L-arginine | DMSO | solution | insufficient solid |
| L-arginine | THF | oil | evaporated to a gum |
| Tromethamine | MeCN/H₂O | solid | matches free acid |
| Tromethamine | IPA | solid | crystalline, Form A |
| Tromethamine | water | solid | matches free acid |
| Tromethamine | DMSO | solution | insufficient solid |
| Tromethamine | THF | solid | partially crystalline, Form A |
| N-ethylglucamine | MeCN/H₂O | solid | weakly crystalline, Form A |
| N-ethylglucamine | IPA | solution | evaporated to solid that |
| | | | deliquesced |
| N-ethylglucamine | water | solid | insufficient solid |
| N-ethylglucamine | DMSO | solution | insufficient solid |
| N-ethylglucamine | THF | oil | evaporated to gum |
| N-methylglucamine | MeCN/H₂O | oil | evaporated to an oil |
| N-methylglucamine | IPA | gel | remained a gel on maturation |
| N-methylglucamine | water | solid | matches free acid |
| N-methylglucamine | DMSO | solution | insufficient solid |
| N-methylglucamine | THF | solid | amorphous, Form A |

Table 5b-part 2

| | | | IC | TGA | Adjusted |
|---|---|---|---|---|---|
| Cation | Solvent | ¹H NMR | acid:base | solvent | IC result |
| Ammonium hydroxide | MeCN/H₂O | shifts seen, water and IPA | 1:0.71 | 20.3% | 1:0.90 |
| Ammonium hydroxide | IPA | shifts seen, water and IPA | 1:0.61 | 8.3% | 1:0.67 |
| Ammonium hydroxide | water | shifts seen, water | 1:0.82 | 11.1% | 1:0.92 |
| Ammonium hydroxide | DMSO | n/a | n/a | n/a | n/a |
| Ammonium hydroxide | THF | shifts seen THF, water, | 1:0.53 | 6.8% | 1:0.57 |
| | | DMF | | | |
| Magnesium acetate | MeCN/H₂O | n/a | n/a | n/a | n/a |
| Magnesium acetate | IPA | shifts seen IPA, water, | 1:0.38 | 14.1% | 1:0.45 |
| | | DMF | | | |
| Magnesium acetate | water | n/a | n/a | n/a | n/a |
| Magnesium acetate | DMSO | n/a | n/a | n/a | n/a |
| Magnesium acetate | THF | shifts seen, water and THF | 1:0.67 | 10.1% | 1:0.74 |
| L-arginine | MeCN/H₂O | shifts seen, water & MeCN | 1:0.97 | 6.5% | 1:1.04 |
| L-arginine | IPA | shifts seen, water and IPA | 1:0.91 | 8.1% | 1:0.99 |
| L-arginine | water | insufficient sample | n/a | n/a | n/a |
| L-arginine | DMSO | n/a | n/a | n/a | n/a |
| L-arginine | THF | n/a | n/a | n/a | n/a |
| Tromethamine | MeCN/H₂O | n/a | n/a | n/a | n/a |
| Tromethamine | IPA | shifts seen IPA, water, ratio | n/a | n/a | n/a |
| | | acid:base 1:1 | | | |
| Tromethamine | water | n/a | n/a | n/a | n/a |
| Tromethamine | DMSO | n/a | n/a | n/a | n/a |
| Tromethamine | THF | shifts seen, water, THF, | n/a | n/a | n/a |
| | | ratio acid:base 1:1.55 | | | |
| N-ethylglucamine | MeCN/H₂O | insufficient solid | n/a | n/a | n/a |
| N-ethylglucamine | IPA | n/a | n/a | n/a | n/a |
| N-ethylglucamine | water | insufficient solid | n/a | n/a | n/a |
| N-ethylglucamine | DMSO | n/a | n/a | n/a | n/a |
| N-ethylglucamine | THF | n/a | n/a | n/a | n/a |
| N-methylglucamine | MeCN/H₂O | n/a | n/a | n/a | n/a |
| N-methylglucamine | IPA | n/a | n/a | n/a | n/a |
| N-methylglucamine | water | n/a | n/a | n/a | n/a |
| N-methylglucamine | DMSO | n/a | n/a | n/a | n/a |
| N-methylglucamine | THF | shifts seen, acid:base ratio | n/a | n/a | n/a |
| | | is 1:0.73, incomplete salt | | | |
| | | formation | | | |

### Scale-Up of Salt Forms

A secondary evaluation of several salt forms was carried out using the methods described above on a 100 mg scale with the results summarized in Table 6 and the figures.

TABLE 6
Scale-up Characterization
Table 6a

| Cation | Solvent | Yield | Purity | XRPD analysis of dry sample | ¹H NMR | TGA |
|---|---|---|---|---|---|---|
| Potassium | THF | 100.3% | 98.8A % | Form D, more crystalline | Shifts seen, salt | 3.4% loss (32-87° C.) |
| hydroxide | | | | | formation confirmed, | 7.8% loss (87-229° C.) |
| | | | | | Residual water, IPA and | |
| | | | | | THF | |
| Sodium | THF | 104.5% | 99.6A % | Form A, more crystalline | Shifts seen, salt | 2.1% loss (32-66° C.) |
| hydroxide | | | | | formation confirmed, | 7.5% loss (66-150° C.) |
| | | | | | Residual water, IPA and | 4.4% loss (150-231° C.) |
| | | | | | THF | 1.6% loss (231-276°) |
| Sodium | IPA | 104.2% | 99.0A % | Consistent with Form A, | Shifts seen, salt | 16.9% loss (32-222° C.) |
| hydroxide | | | | more crystalline | formation confirmed, | 1.5% loss (222-271° C.) |
| | | | | | Residual water, IPA and | |
| | | | | | THF | |
| Sodium | IPA | 103.5% | 97.5A % | Not consistant with Form | Shifts seen, salt | 5.2% loss (31-128° C.) |
| Ethoxide | | | | A or B, similar to Form A | formation confirmed, | 1.4% loss (128-204° C.) |
| | | | | but some key differences | Residual water and IPA | 19.4% loss (204-280° C.) |
| | | | | (called Form C) | | |
| Calcium | IPA | 124.7% | 98.8A % | Form A, more crystalline | Shifts seen, salt | 1.0% loss (31-71° C.) |
| acetate | | | | | formation confirmed, | 8.2% loss (71-217° C.) |
| | | | | | Residual water and IPA | 1.0% loss (217-264° C.) |
| Tromethamine | IPA | 88.6% | 98.7A % | Form A, more crystalline | Shifts seen, salt | 0.8% loss (31-68° C.) |
| | | | | | formation confirmed, | 3.1% loss (68-176° C.) |
| | | | | | ratio acid:base is 1:1.07 | |
| | | | | | i.e. mono salt Residual | |
| | | | | | water and IPA | |
| Ammonium | IPA | 89.7% | 98.1A % | Form A, similar crystallinity | Shifts seen, salt | 1.0% loss (30-80° C.) |
| hydroxide | | | | | formation confirmed, | 4.8% loss (80-165° C.) |
| | | | | | Residual water and IPA | 1.2% loss (165-183° C.) |
| Ammonium | Water | 96.6% | 98.1A % | Form B, less crystalline, some peak | Shifts seen, salt | 8.0% loss (31-115° C.) |
| hydroxide | | | | shifts to smaller 2θ values | formation confirmed, | 1.3% loss (115-173° C.) |
| | | | | | Residual water | 3.8% loss (173-216° C.) |

Table 6b

| | | IC adjusted | | | |
|---|---|---|---|---|---|
| Cation | DSC | for solvent | Aqueous solubility** | GVS | Stability |
| Potassium | Endotherm (onset 25° C., | acid:base | 2.7 mg/ml | n/a | Amorphous after 3 days at |
| hydroxide | 54.4 J/g) Endotherm (onset | 1:1.0 ie | | | 40° C./75% RH |
| | 132° C., 13.6 J/g) | mono salt | | | |
| Sodium | Endotherm (onset 33° C., | acid:base | >10 mg/ml | n/a | Amorphous after 3 days at |
| hydroxide | 22.0 J/g) Endotherm (onset | 1:1.02 ie | | | 40° C./75% RH |
| | 97° C., 17.8 J/g) Endotherm | mono salt | | | |
| | (onset 162° C., 21.8 J/g) | | | | |
| Sodium | Endotherm (onset 88° C., | acid:base | >10 mg/ml | n/a | Amorphous after 3 days at |
| hydroxide | 89.2 J/g) Endotherm (onset | 1:1.11 ie | | | 40° C./75% RH |
| | 256° C., 45.9 J/g) | mono salt | | | |
| Sodium | Endotherm (onset 80° C., | acid:base | 7.7 mg/ml | Total weight change 0-90% RH = | Some changes after 2 days |
| Ethoxide | 17.0 J/g) | 1:0.57 ie | | 8.8 wt %, little hysteresis, | at 40° C./75% RH (no |
| | | hemi salt | | XRPD post GVS shows new | further changes after 10 |
| | | | | form | days at 40° C./75% RH) |
| Calcium | Endotherm (onset 25° C., | acid:base | 0.04 mg/ml | Total weight change 0-90% RH = | No changes after 3 days at |
| acetate | 11.6 J/g) Endotherm (onset | 1:0.84 | | 13 wt %, mostly 80-90% RH, | 40° C./75% RH or a further |
| | 125° C., 79.6 J/g) | | | XRPD post GVS matches | 10 days at 60° C./75% RH |
| | | | | stability samples | |
| Tromethamine | Endotherm (onset 25° C., | n/a | 2.4 mg/ml | Total weight change 0-90% RH = | Some changes after 3 days |
| | 17.6 J/g) Melt endotherm | | | 4.1 wt %, little hysteresis, | at 40° C./75% RH but no |
| | (onset 165° C., 43.7 J/g) | | | XRPD post GVS matches | further changes after 10 |
| | | | | stability samples | days at 60° C./75% RH |
| Ammonium | Endotherm (onset 28° C., | acid:base | 2.3 mg/ml | Total weight change 0-90% RH = | Some changes after 3 days |
| hydroxide | 16.1 J/g) Endotherm (onset | 1:0.56 ie | | 5.3 wt %, sample is hydrated | at 40° C./75% RH but no |
| | 146° C., 63.9 J/g) | hemi salt | | as large wt loss on drying, | further changes after 10 |
| | | | | XRPD post GVS shows no | days at 60° C./75% RH |
| | | | | change | |
| Ammonium | Endotherm (onset 64° C., | acid:base | 1.9 mg/ml | Total weight change 0-90% RH = | No changes after 3 days at |
| hydroxide | 190.9 J/g) Endotherm (onset | 1:0.56 ie | | 6.0 wt %, sample is hydrated | 40° C./75% RH but some |
| | 139° C., 16.7 J/g) Exotherm | hemi salt | | as large wt loss on drying, | changes after a further 10 |
| | (onset 183° C., 14.0 J/g) | | | XRPD post GVS shows new | days at 60° C./75% RH |
| | | | | form | |

Yields have been calculated based on an anhydrous mono salt. Solubility is the aqueous thermodynamic solubility, expressed as free base equivalents

All samples scaled up well, with good chemical purities and yields (though some had residual solvent associated with them). All samples were confirmed to be salts by ¹H NMR.

### Sodium Salts

Both sodium salts are consistent with form A, which confirms reproducibility of form A from the THF solvent system. The IPA/sodium ethoxide method sometimes gave form B but on scale-up the powder pattern was different from both forms A and B, and was called form C. The sodium salts showed good solubility but were not stable to 40° C./75% RH for 3 days.

### Example 9

### Preparation of Solid Form a of Potassium Salt by Recrystallization

Recrystallization: The crude product can be recrystallized either from MeOH or MeOH/EtOH (3:1) by first heating to reflux to dissolve, and then cooling to room temperature to precipitate.

Recrystallization From MeOH: 1.0 g of the potassium salt was suspended in MeOH (150 mL) and heated to reflux for 0.5 h, resulting in an almost clear solution. This was then hot filtered through a Buchner funnel. The clear filtrate on standing at room temperature deposited a white solid. This was stirred overnight and then collected by filtration through a Buchner funnel. The solid product was rinsed with EtOH (2×4.0 mL) and dried in a vacuum oven at 80° C. for 20 h to yield 740 mg of a colorless solid. The mother liquor yielded more title compound on concentration to ca. one-third of the original volume.

Recrystallization from EtOH/MeOH: 1.0 g of the potassium salt was suspended in the solvent mixture EtOH/MeOH (1:3) (200 mL), and heated to reflux for 0.5 h resulting in an almost clear solution. This was then hot filtered through a Buchner funnel. The clear filtrate on standing at room temperature deposited a colorless solid. This was collected by filtration through a Buchner funnel. The solid product was rinsed with EtOH and dried in vacuum oven at 80° C. for 20 h to give a white solid. The mother liquor yielded more title compound upon concentration to ca, one-third of the original volume.

Recrystallization of Form B From MeOH: [4-(6-fluoro-7-methylamino-2,4-dioxo-1,4-dihydro-2H-quinazolin-3-yl)-phenyl]-5-chloro-thiophen-2-yl-sulfonylurea potassium salt (C5 009, 500 mg) was charged to a 100 ml round bottomed flask and methanol (67 ml) added. The suspension was heated with magnetic stirring to reflux for 30 minutes. Dissolution did not occur therefore two further portions of methanol (20 ml) were added over the course of 1 hour. Dissolution had still not occurred and the limits of the vessel had been reached. The suspension was cooled to ambient then filtered under vacuum and the solid (crop 1) was oven dried at 45° C. under vacuum. A portion of the mother liquors (ca. 20 ml) was concentrated under vacuum to dryness (crop 2) and the remaining mother liquors were concentrated to ca. 30 ml. Within minutes, it was observed that the flask became very cold and much solid precipitated (crop 3). This suggested that the solution was not saturated before concentration.

XRPD analysis of all three crops showed that only crop 3 resembled the form A powder pattern exactly. It was hypothesised that crops 1 and 2 were solids in transition between form B and form C, as crop 1 appeared to contain the 5.2 2Theta peak that is distinctive of form B, and crop 2 did not have the form B peak, but neither did it have the 4.8 2Theta form A peak. A single crystal from the liquors of crop 3 confirmed that form A is a 2.5 hydrate where one molecule of water is coordinated to the potassium and for each [4-(6-fluoro-7-methylamino-2,4-dioxo-1,4-dihydro-2H-quinazolin-3-yl)-phenyl]-5-chloro-thiophen-2-yl-sulfonylurea potassium salt moiety, 1.5 molecules of water are hydrogen bonded. It is thought that the ease of movement of the hydrogen bonded water determines whether the peak at 4.8 2Theta is observed or not. The structure details can be found in Table 18.

### Example 10

### Preparation of Form B of Potassium Salt by Recrystallization

Recrystallization: The crude product can be recrystallized from EtOH/H₂O (91:9) or a small volume of MeOH by first heating to reflux to dissolve, and then cooling to room temperature to precipitate.

Recrystallization from EtOH/H₂O: 1.0 g of the potassium salt was suspended in EtOH (190 mL) and heated to reflux. To the heavy suspension was added H₂O (18.0 mL) dropwise, resulting in a clear colorless solution. On cooling to room temperature, the title compound precipitated out as a white solid. It was collected by filtration through a Buchner funnel, and rinsed with EtOH (2×4.0 mL). This was dried in vacuum oven at 80° C. for 20 h, to give 650 mg of a colorless solid. The mother liquor yielded more title compound upon concentration to ca. one-third of the original volume.

Large Scale recrystallization from small volume of MeOH: 6.6 g of the potassium salt was suspended in MeOH (30 mL) and heated to reflux for 5 hr, the solid did not completely dissolve in this volume of methanol. After cooling the solid was filtered and rinsed with iPrOH. The solid was dried in vacuum oven at 80° C. for 20 h, to give 6.2 g of colorless solid, which after characterization was shown to be form B.

Form B has been shown to be quite stable towards moisture and temperature. The API has been exposed to 75% RH/40° C. for up to 6 months with no change in solid state.

### Example 11

### Polymorphism Studies on Form B of the Potassium Salt

The propensity of [4-(6-fluoro-7-methylamino-2,4-dioxo-1,4-dihydro-2H-quinazolin-3-yl)-phenyl]-5-chloro-thiophen-2-yl-sulfonylurea potassium salt form B to form polymorphs was studied. Form B (a hemi-hydrate) was slurried in a range of solvents (neat and mixtures). The solvents were chosen based on their pharmaceutical acceptability and also a range of functional groups and polarities such as alcohols, ethers and esters. To encourage hydrate formation, aqueous mixtures were also chosen. The solvents used are detailed in Table 7.

TABLE 7
Ambient polymorphism experiments

| solvent | volume/μl | XRPD |
|---|---|---|
| acetone | 500 | Form B |
| acetone/water | 500 | change in pattern |
| THF | 500 | Form B |
| THF/water | 500 | mixture of Form B and pattern of |
| | | solid from acetone/water |
| EtOH | 500 | Form B |
| EtOH/water | 500 | Form B |
| DCM | 500 | Form B |
| DCM/MeOH (9:1) | 500 | Form B |
| MtBE | 500 | Form B |
| 2-MeOEtOH | 500 | this solvent dissolved K salt |
| 2-MeOEtOH/water | 500 | Form B |
| dioxane | 500 | Form B |
| dioxane/water | 500 | Form B |
| MEK | 500 | Form B |
| IPA | 500 | Form B |
| IPA/water | 500 | Form B |
| EtOAc | 500 | Form B |
| EtOAc/heptane | 500 | Form B |
| MeCN | 500 | Form B |
| MeCN/water | 500 | Form B |
| water | 500 | Form B |

Approximately 50 mg of [4-(6-fluoro-7-methylamino-2,4-dioxo-1,4-dihydro-2H-quinazolin-3-yl)-phenyl]-5-chloro-thiophen-2-yl-sulfonylurea potassium salt, form B was suspended in ten volumes of the solvents detailed in table 7 and stirred at ambient for two hours. It was observed that 2-methoxyethanol was the only solvent that dissolved the potassium salt. The suspensions were filtered under vacuum and analysed by XRPD. Most of the solids remained as form B, with a 1:1 acetone/water mixture leading to a subtle change in solid form. The 1:1 tetrahydrofuran/water mixture generated a mixture of that subtly different form and form B.

To all the form B samples a further aliquot of five volumes of appropriate solvent was added and the suspensions were slurried at 50° C. for 4 hours then cooled to ambient for 4 hours. This cycle was repeated for a total of 24 hours, after which time the suspensions were filtered under vacuum and analysed by XRPD. The results are detailed in Table 8.

TABLE 8
Heat cycle polymorphism experiments

| | volume/μl | |
|---|---|---|
| | (further | |
| solvent | portion) | XRPD |
| acetone | 250 | amorphous |
| acetone/water | 250 | n/a |
| THF | 250 | amorphous |
| THF/water | 250 | n/a |
| EtOH | 250 | family 2 |
| EtOH/water | 250 | family 1 |
| DCM | 250 | family 2 |
| DCM/MeOH (9:1) | 250 | family 2 |
| MtBE | 250 | Form B |
| 2-MeOEtOH | 250 | n/a |
| 2-MeOEtOH/water | 250 | family 3 |
| dioxane | 250 | family 4 |
| dioxane/water | 250 | family 5 |
| MEK | 250 | family 2 |
| IPA | 250 | family 2 |
| IPA/water | 250 | family 1 |
| EtOAc | 250 | family 2 |
| EtOAc/heptane | 250 | family 2 |
| MeCN | 250 | Form B |
| MeCN/water | 250 | family 1 |
| water | 250 | Form B |

The changes observed in solid form were only slightly different from form B. For this reason, the different phases were categorised into families rather than given definitive form names until further analysis had confirmed them as being different.

In order to characterise the materials, a range of techniques (DSC, VTXRPD and ¹H NMR) were carried out.

### Identification of Family 1

The powder pattern of family 1 was the best match for form B of all the families isolated. The only differences appeared to be due to reduction in resolution (probably due to the instrument used). To confirm that this was the case thermal analysis was carried out. The DSC showed that the form B starting material melted slightly lower than the family 1 sample. To deduce if this was due to impurities, purity analysis was carried out on both samples.

The purity analysis measured the family 1 sample to be 99.8 area % and the foam B starting material to be 99.9%. Purity was therefore ruled out as a reason for the difference. It was decided to carry out a VT XRPD experiment to deduce what the desolvated phase was. However, the solid when reanalysed had converted completely to form B. Family 1 was therefore not re-investigated.

### Identification of Family 2

The phase labelled family 2 was isolated from many of the solvent systems used. In order to deduce whether or not the phase was a hydrate, thermal analysis was carried out. The DSC experiment showed an endotherm suspected to be associated with a desolvation from ambient to ca. 102° C. This desolvated phase then melted at 281° C. Karl Fischer analysis confirmed 3.4% water content which is equivalent to 1.1 moles. To obtain further sample for the stabilities studies, a further aliquot of the original suspension was filtered. However, the XRPD showed the distinctive 5.2 2Th peak which was indicative that the sample was changing to form B. A DSC experiment was run to confirm the melting point, and it appeared that the sample was a mixture of form B (hemi-hydrate) and the mono hydrate, as the melting point had been reduced almost to that of form B at 279° C. from 281° C.

### Identification of Family 3

This solid form was isolated from 2-MeOEtOH/H₂O (1:1), as were single crystals generated in a separate experiment. The single crystal structure was solved as being a hemi 2-methoxy ethanol solvate, hemi hydrate and it was found that the calculated powder pattern from the data was very close to the actual pattern of form B. The structure showed that the water molecules were in the coordination sphere of the potassium. However, the 2-Methoxy ethanol was interacting via hydrogen bonding. It was thought that the 2-methoxy ethanol could pass in and out of the structure without causing any change to it, i.e. resulting in a de-solvated solvate, hence the similar powder patterns.

### Identification of Family 4

The solid labelled as family 4 was the only solid isolated of this form. The DSC analysis indicated a desolvation from a broad endotherm that occurred from an onset of 25° C. to ca. 130° C. After this transition the trace was representative of an amorphous phase. It was hypothesised if this form was in fact a solvate that de-solvated to an amorphous phase. To confirm this, a VT-XRPD experiment was carried out.

The polymorphism screen concluded that form B (a hemi hydrate) showed propensity for further hydration or solvation. It was also noted that when further solvated by 2-methoxy ethanol, the solvent filled channels (detailed below).

### 2-Methoxy Ethanol/Water Crystallisations

A number of re-crystallisations were carried out using 2-methoxy ethanol and water as co-solvent as it had been deduced that 2-methoxy ethanol was the only solvent other than dimethylsulfoxide that dissolved [4-(6-fluoro-7-methylamino-2,4-dioxo-1,4-dihydro-2H-quinazolin-3-yl)-phenyl]-5-chloro-thiophen-2-yl-sulfonylurea potassium salt. The following reactions were carried out:

TABLE 9

| Solvent | Recrystallisation | Experimental | XRPD | ¹H NMR |
|---|---|---|---|---|
| system | conditions | observations | results | results |
| 2- | Form B | Solid dissolved on | Has the same | Insufficient |
| MeOEtOH/ | suspended in 10 | heating, but | powder pattern | material for |
| H₂O (1:1) | volumes and | material | as form B, | analysis |
| | heated with | crystallized in | although | |
| | stirring to 93° C. | minutes without | suspected to be | |
| | with magnetic stirring. | cooling required. | an isostructrual | |
| | | | 2-methoxy | |
| | | | ethanol solvate. | |
| 2- | Form B | Solid dissolved on | Not applicable | Not applicable |
| MeOEtOH/ | suspended in 20 | heating, but did | | |
| H₂O (60:40) | volumes and | not crystallize on | | |
| | heated to 70° C. | cooling. Oil | | |
| | with magnetic stirring | observed after 6 days. | | |
| 2- | Form B | Solid dissolved on | Very close to | 0.68 moles of |
| MeOEtOH/ | suspended in 20 | heating and | form B, but | 2-methoxy |
| H₂O (1:1) | volumes and | crystallized on | suspected to be | ethanol |
| | heated to 73° C. | cooling. | an isostructrual | integrated. |
| | with magnetic stirring. | | 2-methoxy | Unstable |
| | | | ethanol solvate. | solvates |
| | | | | containing |
| | | | | slightly |
| | | | | different |
| | | | | amounts of 2- |
| | | | | methoxy |
| | | | | ethanol |
| 2- | Form B | Solid dissolved on | Very close to | 0.49 moles of |
| MeOEtOH/ | suspended in 15 | heating and | form B, but | 2-methoxy |
| H₂O (60:40) | volumes and | crystallised on | suspected to be | ethanol |
| | heated to 73° C. | cooling. | an isostructrual | integrated. |
| | with magnetic stirring. | | 2-methoxy | Unstable |
| | | | ethanol solvate. | solvates |
| | | | | containing |
| | | | | slightly |
| | | | | different |
| | | | | amounts of 2- |
| | | | | methoxy |
| | | | | ethanol |

In order to confirm that the desolvation of the 2-methoxy ethanol solvate to the hemi hydrate (that has been called form B to date) does not cause a significant change in the structure, and hence the powder pattern, a VT XRPD was carried out and the solid re-analysed by ¹H NMR. It was deduced that 2-methoxy ethanol/water combinations could not generate any form other than a 2-methoxy ethanol solvate of the hemi hydrate form B. It was therefore ruled out as a potential recrystallisation solvent due to it being regarded as a class II (ICH guidelines) solvent and therefore having residual level limits of 50 ppm.

### Potassium Salt Formation From [4-(6-fluoro-7-methylamino-2,4-dioxo-1,4-dihydro-2H-quinazolin-3-yl)-phenyl]-5-chloro-thiophen-2-yl-sulfonylurea free acid

A selection of solvents and aqueous solvent combinations that gave rise to subtle differences from form B in the polymorphism screen were chosen as reaction solvents for generating the potassium salt from the free acid. The following experiments were carried out:

TABLE 10
Experimental observations and results

| | | | Observations | | |
|---|---|---|---|---|---|
| | | | after ca. 5 | Observations | |
| | Experimental | Experimental | minutes at | on cooling to | XRPD |
| Solvent | conditions | observations | 50° C. | ambient | result |
| acetone/water | Free acid | KOH added to | Suspension | Suspension | Form B |
| (1:1) | suspension | suspensions. | | | |
| | heated to 50° C. | Most solid | | | |
| | with stirring in | dissolved | | | |
| ethanol | 10 volumes then | KOH added to | Suspension | Suspension | Form B |
| | KOH (1.0 equ as | suspensions. | | | |
| | 1M in H₂O) added. | Suspension | | | |
| ethanol/water | | KOH added to | Suspension | Suspension | Form B |
| (1:1) | | suspensions. | | | |
| | | Most solid | | | |
| | | dissolved | | | |
| dioxane | | KOH added to | Suspension | Suspension | New |
| | | suspensions. | | | pattern |
| | | Suspension | | | |
| dioxane/water | | KOH added to | Solution | Solution | n/a |
| (1:1) | | suspensions. | | | |
| | | Most solid | | | |
| | | dissolved | | | |

The four suspensions were filtered under vacuum and air dried. XRPD analysis was then carried out. The sample from dioxane/water was discarded after one week as a brown oil was present. The solid from dioxane that gave the new powder pattern was fully characterised and deduced to be a 1,4-dioxane solvate with two equivalents of solvent to one of [4-(6-fluoro-7-methylamino-2,4-dioxo-1,4-dihydro-2H-quinazolin-3-yl)-phenyl]-5-chloro-thiophen-2-yl-sulfonylurea.

The experiments carried out have shown that when starting with form B (dried of weakly bound solvent to a hemi hydrate), the solid further hydrates to a mono hydrate or solvates with certain solvents. The solvents fill a channel which therefore causes no change in structure when the solvent molecules vacate the spaces. For this reason, techniques other than XRPD alone are needed to deduce the actual form isolated. For further development of form B, it must be confirmed that the material has been dried sufficiently to the hemi hydrate. No anhydrous forms of [4-(6-fluoro-7-methylamino-2,4-dioxo-1,4-dihydro-2H-quinazolin-3-yl)-phenyl]-5-chloro-thiophen-2-yl-sulfonylurea potassium salt were identified.

### Example 12

### Preparation of Form C of Potassium Salt by Wet Granulation

A change in solid phase from form B was identified when wet granulation was carried out. Thus grinding form B of [4-(6-fluoro-7-methylamino-2,4-dioxo-1,4-dihydro-2H-quinazolin-3-yl)-phenyl]-5-chloro-thiophen-2-yl-sulfonylurea potassium salt with 75% and 90% w/w water using mortar and pestle followed by heating at 40° C. overnight results in conversion to either an amorphous form or a new form—form C of [4-(6-fluoro-7-methylamino-2,4-dioxo-1,4-dihydro-2H-quinazolin-3-yl)-phenyl]-5-chloro-thiophen-2-yl-sulfonylurea potassium salt. Form C has XRPD and DSC properties which are different from forms A and B of [4-(6-fluoro-7-methylamino-2,4-dioxo-1,4-dihydro-2H-quinazolin-3-yl)-phenyl]-5-chloro-thiophen-2-yl-sulfonylurea potassium salt. This new form also resulted from a wet granulation process where the API was mixed with excipients including Avicel, triacyl citrate, and water in a low shear granulator followed by extrusion and spherinization. In addition, this new form was possible to make in aqueous slurry when stored at ambient room temperature or in a refrigerator (2-8° C.) for prolonged periods, i.e., 3 days.

The sample (primarily referred to as form C) was characterised by cation chromatography to confirm that the potassium salt was intact. The measurement confirmed 0.92 equivalents of potassium to [4-(6-fluoro-7-methylamino-2,4-dioxo-1,4-dihydro-2H-quinazolin-3-yl)-phenyl]-5-chloro-thiophen-2-yl-sulfonylurea, which was corrected for solvent content deduced by TGA. This new form C was subsequently identified to be a hemi-potassium salt of [4-(6-fluoro-7-methylamino-2,4-dioxo-1,4-dihydro-2H-quinazolin-3-yl)-phenyl]-5-chloro-thiophen-2-yl-sulfonylurea.

TABLE 11
Aqueous solubility measurements of form C

| Thermodynamic solubility in | |
|---|---|
| water/mg · ml⁻¹ | pH |
| 4.5 | 8.7 |
| 4.5 | 8.8 |

On an 800 mg scale and 90% volume of water, form B was ground in a glass mortar with 90% volume of water with both phosphate buffer (pH 7.4 made form H₃PO₄ and KOH) and DI water for between five and ten minutes. Samples were reanalysed by XRPD post grinding.

TABLE 12
Manual grinding experiments

| Experiment | XRPD result |
|---|---|
| Form B ground in a large | Form B |
| glass mortar with 90% | |
| volume of water for ca. 5 | |
| minutes. An aliquot taken | |
| and stored at 4° C. for 4 days. | |
| Form B ground in a large | Form B |
| glass mortar with 90% | |
| volume of water for ca. 5 | |
| minutes. The paste was | |
| spread on a glass slide and air | |
| dried. | |
| A further 90% volume of | Form B (much reduced |
| water added to the bulk | crystallinity) |
| sample and ground for ca. 5 | |
| minutes. The paste was | |
| spread on a glass slide and | |
| dried at 45° C. | |
| A further 90% volume of | Very close in pattern to form |
| water added to the bulk | C |
| sample and ground for ca. 5 | |
| minutes. The sample stored | |
| at 45° C. | |

The conclusion was that if form B was ground sufficiently to break down the lattice and the amorphous phase was in the presence of water, it would hydrate to form C. To gain further information on the relative stabilities of form B and form C a number of experiments were set up involving 1:1 mixtures of the solids.

### Qualitative Relative Stability Studies

The relative stability of form A (a dihydrate) with form B and form C was studied.

### Qualitative Relative Stability Studies Carried Out on Form B and Form C

Approximately a 1:1 ratio of form B and form C were lightly ground together in an agate mortar and a powder pattern was obtained. The following experiments were carried out on the mixture.

TABLE 13
Relative stability experiments

| | XRPD result | XRPD result |
|---|---|---|
| Experiment | after 1 day | after 4 days |
| The mixture was | The sample was | Solid had |
| suspended in | an emulsion | crystallised from |
| water (500 μl) | therefore | the dried |
| and stirred | pipetted onto a | emulsion to be |
| magnetically. | glass slide to | form C |
| | dry. | |
| The mixture was | Mixture | Mixture |
| stood in an | | |
| atmosphere of | | |
| 75% RH at | | |
| 25° C. | | |
| The mixture was | Mixture | Mixture |
| stood in | | |
| standard lab | | |
| conditions with | | |
| nothing added to | | |
| it. | | |

These results supported that it was the amorphous potassium salt that crystallized as form C.

Form C from Form B

In order to deduce if there was a robust method of converting form B to form C, a series of experiments were carried out using different lots of [4-(6-fluoro-7-methylamino-2,4-dioxo-1,4-dihydro-2H-quinazolin-3-yl)-phenyl]-5-chloro-thiophen-2-yl-sulfonylurea potassium salt. The differences between lots were in particle size. The experiments were set up, the suspensions were filtered and washed with water and the results are detailed in Table 14.

TABLE 14
Experimental procedure and results

| | | | | XRPD |
|---|---|---|---|---|
| Compound | Amount of | Volume of | Temperature of | result post |
| 7a (k salt) | salt/mg | H₂O | suspension/° C. | filtration |
| not milled | ca. 50 | 3.6 | ambient | mixture of B |
| | | | | and C |
| not milled | ca. 50 | 3.6 | 4 | mixture of B |
| | | | | and C |
| not milled | ca. 50 | 3.6 | 50 | B |
| not milled | ca. 50 | 5 | ambient | B |
| not milled | ca. 50 | 5 | 4 | B |
| milled | ca. 50 | 5 | ambient | mixture of B |
| | | | | and C |
| milled | ca. 50 | 5 | 4 | mixture of B |
| | | | | and C |
| milled | ca. 50 | 5 | 50 | mixture of B |
| | | | | and C |

From the nine experiments carried out, eight were confirmed as form B or mixtures of form B and C and one led to single crystals that were of sufficient quality for diffraction. The crystal structure was solved as a hemi potassium salt which was hydrated. The level of hydration was difficult to confirm due to the water being held in channels that allowed for easy desolvation. It is currently thought that at full occupancy 3 moles of water are present (see Table 19 for details).

### Qualitative Relative Stability Studies Carried Out on Form A and Form C

Approximately a 1:1 ratio of form A and the form C were lightly ground together in an agate mortar and a powder pattern was obtained.

TABLE 15
Relative stability experiments

| Experiment | XRPD results after 4 days |
|---|---|
| Form A/C mixture exposed | No change from mixture |
| to a 40° C./75% RH | |
| atmosphere | |
| Form A/C mixture stored in a | No change from mixture |
| 60° C./75% RH atmosphere for | |
| 5 days | |

The stressing conditions caused no conversion to either form.

### Example 13

### Single Crystal X-Ray Diffraction Studies

Four samples were submitted for single crystal X-ray diffraction studies. The resulting structure analyses are provided throughout the rest of this section.

TABLE 16
Single crystal structure of [4-(6-fluoro-7-methylamino-2,4-dioxo-
1,4-dihydro-2H-quinazolin-3-yl)-phenyl]-5-chloro-thiophen-2-yl-
sulfonylurea potassium salt hemi 2-methoxy ethanol solvate,
hemi hydrate

| Molecular formula | C_{21·50}H₁₉ClFKN₅O_{6 ·50}S₂ | | | | |
|---|---|---|---|---|---|
| Molecular weight | 609.09 | | | | |
| Crystal system | Monoclinic | | | | |
| Space group | C2/C | a | 33.3580(5) Å, | α | 90°, |
| | | b | 15.093(3) Å, | β | 92.0408(7)°, |
| | | c | 20.0081(4) Å, | γ | 90° |

| | |
|---|---|
| V | 10067(2) Å³ |
| Z | 16 |
| D_{c} | 1.607 g cm⁻¹ |
| μ | 0.542 mm⁻¹ |
| Source, λ | Mo—Kα, 0.71073 Å |
| F(000) | 4992 |
| T | 120(1)K |
| Crystal | Colourless, 0.4 × 0.4 × 0.05 mm |
| Data truncated to | 0.80 Å |
| θₘₐₓ | 22.44° |
| Completeness | 99.9% |
| Reflections | 26301 |
| Unique reflections | 10284 |
| Rᵢₙₜ | 0.0525 |

The structure solution was obtained by direct methods, full-matrix least-squares refinement on F² with weighting w⁻¹=σ²(Fₒ²)+(0.0925P)²+(20.0000P), where P=(Fₒ²+2F_{c}²)/3, anisotropic displacement parameters, no absorption correction. Final wR²={Σ[w(Fₒ²−F_{c}²)²]/Σ[w(Fₒ²)²]^{1/2}}=0.1621 for all data, conventional R₁=0.0514 on F values of 7471 reflections with Fₒ>4σ(Fₒ), S=1.002 for all data and 708 parameters. Final Δ/σ(max) 0.005, Δ/σ(mean), 0.000.

TABLE 17
Single crystal structure of mono acetonitrile solvate, hemi hydrate

| Molecular formula | C_{21·50}H₁₉ClFKN₅O_{6·50}S₂ | | | | |
|---|---|---|---|---|---|
| Molecular weight | 609.09 | | | | |
| Crystal system | Monoclinic | | | | |
| Space group | C₂/c | a | 33.6106(5) Å, | α | 90°, |
| | | b | 15.0902(3) Å, | β | 91.8800(10)°, |
| | | c | 20.1282(3) Å, | γ | 90° |

| | |
|---|---|
| V | 10203.3(3) Å³ |
| Z | 16 |
| D_{c} | 1.586 g cm⁻¹ |
| μ | 0.535 mm⁻¹ |
| Source, λ | Mo—Kα, 0.71073 Å |
| F(000) | 4992 |
| T | 120(1)K |
| Crystal | colourless, 0.4 × 0.4 × 0.05 mm |
| Data truncated to | 0.80 Å |
| θₘₐₓ | 22.44° |
| Completeness | 99.9% |
| Reflections | 45568 |
| Unique reflections | 10424 |
| Rᵢₙₜ | 0.0679 |

The structure solution was obtained by direct methods, full-matrix least-squares refinement on F² with weighting w⁻¹=σ²(Fₒ²)+(0.1000P)²+(0.0000P), where P=(Fₒ²+2F_{c}²)/3, anisotropic displacement parameters, no absorption correction. Final wR²={Σ[w(Fₒ²−F_{c}²)²]/Σ[w(Fₒ²)²]^{1/2}}=0.1808 for all data, conventional R₁=0.0567 on F values of 7073 reflections with Fₒ>4σ(Fₒ), S=1.154 for all data and 721 parameters. Final Δ/σ(max) 0.003, Δ/σ(mean), 0.000.

TABLE 18
Single crystal structure of [4-(6-fluoro-7-methylamino-2,4-dioxo-
1,4-dihydro-2H-quinazolin-3-yl)-phenyl]-5-chloro-thiophen-2-yl-
sulfonylurea potassium salt 2.5 hydrate (form A)

| Molecular formula | C₂₀H₂₀ClFKN₅O_{7·50}S₂ | | | | |
|---|---|---|---|---|---|
| Molecular weight | 608.08 | | | | |
| Crystal system | Monoclinic | | | | |
| Space group | P2₁/n | a | 21.1534(5) Å, | α | 90°, |
| | | b | 6.9137(2) Å, | β | 93.774(2)°, |
| | | c | 34.8001(11) Å, | γ | 90° |

| | |
|---|---|
| V | 5078.4(2) Å³ |
| Z | 8 |
| D_{c} | 1.591 g cm⁻¹ |
| μ | 0.54 mm⁻¹ |
| Source, λ | Mo—Kα, 0.71073 Å |
| F(000) | 2496 |
| T | 120(1)K |
| Crystal | colourless prism, 0.16 × 0.12 × 0.05 mm |
| θₘₐₓ | 22.47° |
| Completeness | 91.5% |
| Reflections | 12824 |
| Unique reflections | 6056 |
| Rᵢₙₜ | 0.0497 |

The structure solution was obtained by direct methods, full-matrix least-squares refinement on F² with weighting w⁻¹=σ²(Fₒ²)+(0.1000P)²+(0.0000P), where P=(Fₒ²+2F_{c}²)/3, anisotropic displacement parameters, no absorption correction. Final wR²={Σ[w(Fₒ²−F_{c}²)²]/Σ[w(Fₒ²)²]^{1/2}}=0.2072 for all data, conventional R₁=0.0636 on F values of 4777 reflections with Fₒ>4σ(Fₒ), S=1.493 for all data and 678 parameters. Final Δ/σ(max) 0.01, Δ/σ(mean), 0.001.

TABLE 19
Single crystal structure of [4-(6-fluoro-7-methylamino-2,4-dioxo-
1,4-dihydro-2H-quinazolin-3-yl)-phenyl]-5-chloro-thiophen-2-yl-
sulfonylurea potassium salt hemi-potassium salt, hydrate, (form C)

| Molecular formula | C₂₀H₁₄ClFK_{0·50}N₅O₅S_{2.}xH₂O (x = ca. 5) | | | | |
|---|---|---|---|---|---|
| Molecular weight | 542.48 | | | | |
| Crystal system | Triclinic | | | | |
| Space group | P-1 | a | 11.2838(6) Å, | α | 117.623(4)°, |
| | | b | 11.4461(6) Å, | β | 94.376(3)°, |
| | | c | 11.7629(7) Å, | γ | 98.599(3)° |

| | |
|---|---|
| V | 1312.43(13) Å³ |
| Z | 2 |
| D_{c} | 1.373 g · cm⁻¹ |
| μ | 0.429 mm⁻¹ |
| Source, λ | Mo—Kα, 0.71073 Å |
| F(000) | 553 |
| T | 180(2)K |
| Crystal | colourless plate, 0.12 × 0.12 × 0.02 mm |
| Data truncated to | 0.80 Å |
| θₘₐₓ | 22.44° |
| Completeness | 99.1% |
| Reflections | 9120 |
| Unique reflections | 3370 |
| Rᵢₙₜ | 0.0577 |

The structure solution was obtained by direct methods, full-matrix least-squares refinement on F² with weighting w⁻¹=σ²(Fₒ²)+(0.1500P)²+(3.5000P), where P=(Fₒ²+2F_{c}²)/3, anisotropic displacement parameters, no absorption correction. Final wR²={Σ[w(Fₒ²−F_{c}²)²]/Σ[w(Fₒ²)²]^{1/2}}=0.2571 for all data, conventional R₁=0.0778 on F values of 2459 reflections with Fₒ>4σ(Fₒ), S=1.069 for all data and 368 parameters. Final Δ/σ(max) 0.004, Δ/σ(mean), 0.000. Final difference map between +1.143 and −0.685e·Å⁻³.

### Example 14

### Preparation of Polymorph Form D of Potassium Salt by Recrystallization

Method: 40 volumes of THF was added to 100 mg of free acid at room temperature. This was then heated to 50° C. for 2 hours and cooled at 4° C. slowly. The solid was filtered and dried in a vacuum oven at 25° C. The solid was confirmed to the mono potassium salt by ion chromatography.

Although the foregoing invention has been described in some detail by way of illustration and example for purposes of clarity of understanding, one of skill in the art will appreciate that certain changes and modifications may be practiced within the scope of the appended claims. In addition, each reference provided herein is incorporated by reference in its entirety to the same extent as if each reference was individually incorporated by reference.

1. A salt comprising a compound Formula I: 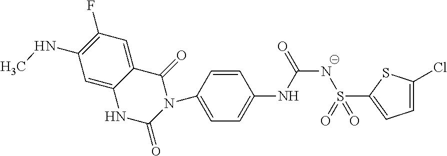
and an ion selected from the group consisting of sodium, potassium, calcium, L-lysine, ammonium, magnesium, L-arginine, tromethamine, N-ethylglucamine and N-methylglucamine.

2.-9. (canceled)

10. A salt having the formula: 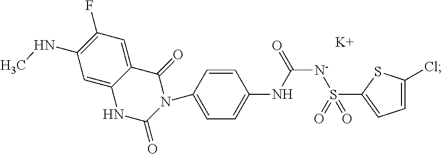
in a crystalline solid form C characterized by at least one of:
(i) an X-ray powder diffraction pattern substantially in accordance with FIG. 15; and
(ii) a DSC scan substantially in accordance with the DSC pattern shown in FIG. 20.

11.-12. (canceled)

13. A salt having the formula: 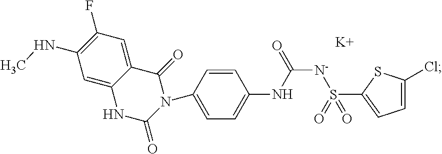
in a crystalline solid form D characterized by at least one of:
(i) an X-ray powder diffraction pattern substantially in accordance with FIG. 21; and
(ii) a DSC scan substantially in accordance with the DSC pattern shown in FIG. 24.

14.-15. (canceled)

16. A salt having the formula: 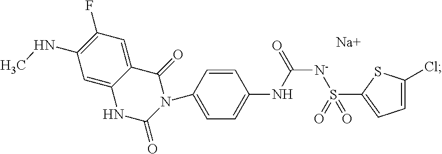
in a crystalline solid form A which provides at least one of:
(i) an X-ray powder diffraction pattern substantially in accordance with FIG. 25; and
(ii) a DSC scan substantially in accordance with FIG. 29.

17.-18. (canceled)

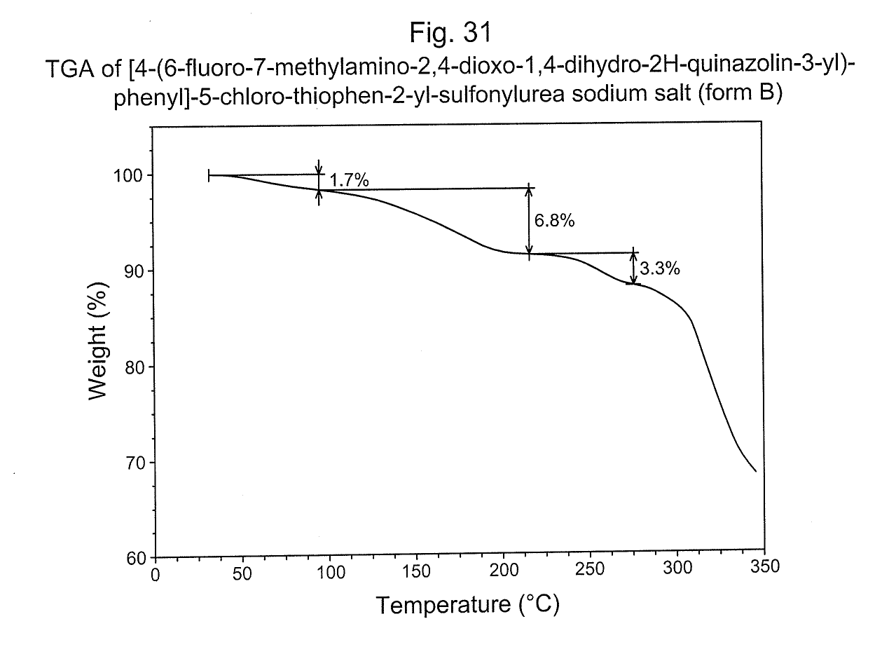

19. A salt having the formula: 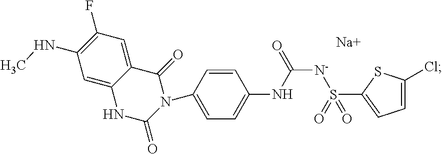
in a crystalline solid form B which provides at least one of:
(i) an X-ray powder diffraction pattern substantially in accordance with FIG. 30; and
(ii) a TGA scan substantially in accordance with FIG. 31.

20. (canceled)

21. A salt having the formula: 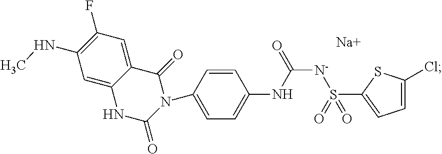
in a crystalline solid form C which provides at least one of:
(i) an X-ray powder diffraction pattern substantially in accordance with FIG. 32; and
(ii) a DSC scan substantially in accordance with FIG. 34.

22.-23. (canceled)

24. A salt having the formula: 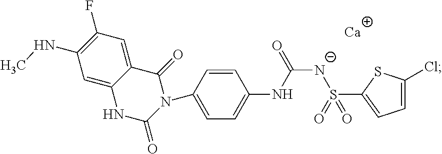
in a crystalline solid form A which provides at least one of:
(i) an X-ray powder diffraction pattern substantially in accordance with FIG. 37; and
(ii) a DSC scan substantially in accordance with FIG. 43.

25.-26. (canceled)

27. A salt having the formula: 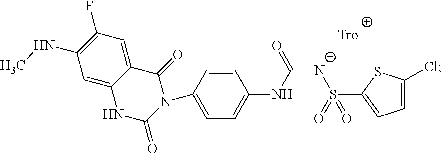
in a crystalline solid form A which provides at least one of:
(i) an X-ray powder diffraction pattern substantially in accordance with FIG. 44; and
(ii) a DSC scan substantially in accordance with FIG. 48.

28.-30. (canceled)

31. A pharmaceutical composition comprising a therapeutically effective amount of a compound according to claim 1 and a pharmaceutically acceptable vehicle or carrier.

32.-42. (canceled)

43. A pharmaceutical composition comprising a therapeutically effective amount of a compound according to claim 1 and an additional therapeutic agent.

44. (canceled)

45. A pharmaceutical composition for preventing or treating a condition in a mammal characterized by undesired thrombosis comprising a pharmaceutically acceptable carrier and a therapeutically effective amount of a salt of claim 1.

46. A method of preparing a salt of formula I: 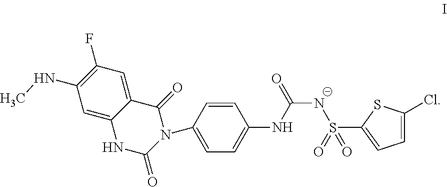
comprising contacting a base with a compound of formula II: 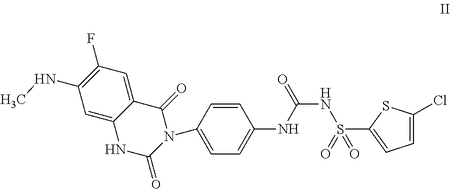
or a salt thereof under conditions to form the salt of formula I.

47.-51. (canceled)

52. A method for preventing or treating thrombosis and thrombosis related conditions in a mammal comprising the step of administering to a mammal a therapeutically effective amount of a salt of claim 1.

53. A method for preventing or treating a condition or disorder mediated at least in part by ADP-induced platelet aggregation in a mammal comprising the step of administering to a mammal in need of such treatment in a therapeutically effective amount of a composition of claim 1 or a pharmaceutically acceptable salt thereof.

54. A method for inhibiting the coagulation of a blood sample comprising the step of contacting said sample with said salt a salt of claims 1.

55.-64. (canceled)

65. A method for preventing the occurrence of a secondary ischemic event comprising administering to a patient who has suffered a primary ischemic event a therapeutically effective amount of a salt of claim 1, together with a pharmaceutically acceptable carrier.

66.-67. (canceled)

68. A method for the preparation of a pharmaceutical composition comprising admixing a therapeutically effective amount of the salt of claim 1 with a pharmaceutically acceptable vehicle or carrier.
